SEQUENCE LISTING (30 sequences)

SEQ ID NO: 1 - DNA, 361 bp, Homo sapiens
  source 1..361: mol_type = other DNA
gaggttcagctggtggagtctgggggaggcgtggtccagcctggggggtccctgagactctcctgtgcagcgtctggattcaccttcagtaactatggcatgcactgggtccgccaggctccaagcaaggggctggagtgggtggcctttatacggtatgatggaactaataaatactatgcagactccgtgaagggccgattcaccatctccagagacaattccaagaacacgttgtatctgcaaatcaacagcctgagagctgaggacacggctgtgtattactgtgcgattatagcagtggctggcgatgtcaaccctgactactggggccagggaaccctggtcaccgtctcctcag

SEQ ID NO: 2 - DNA, 331 bp, Homo sapiens
  source 1..331: mol_type = other DNA
tcttatgagctgactcagccaccctcagcgtctgggacccccgggcagagggtcaccatctcttgttctggaagcagctccaacatcggaagtaatactgtaaactggtaccagcagctcccaggaacggcccccaaactcctcatctatagtaataatcagcggccctcaggggtccctgaccgattctctggctccaagtctgacacctcagcctccctggccatcagtgggctccagtctgaggatgaggctgattattattgtgcagcatgggatgacagcctgaatggttgggtgttcggcggagggaccaagctgaccgtcctag

SEQ ID NO: 3 - DNA, 355 bp, Homo sapiens
  source 1..355: mol_type = other DNA
gaggtgcagctggtggagtctgggggaggcttggtacagccagggcggtccctgagactctcctgtacagcttctggattcacctttggtgatcattctatgagctgggtccgccaggctccagggaaggggctggagtgggtaggtttcattagaagcaaagcttatggtgggacaacagaatatgccgcgtctgtgaaaggcagattcaccatctcaagagatgattccaagagcatcgcctatttgcaaatgaacagcctgaaaatcgaggacacagccgtgtattactgtactagaggactcacggggctcgactactggggccagggaatgctggtcaccgtctcctcag

SEQ ID NO: 4 - DNA, 340 bp, Homo sapiens
  source 1..340: mol_type = other DNA
gacattgtgctgacccagtctccagactccctggctgtgtctctgggcgagagggccaccatcaactgcaagtccagccagagtcttttatacagctccaacaataagaactacttagcttggtaccagcagaaaccaagacagcctcctaagctgctcatttactgggcatctacccgggaatccggggtccctgaccgattcagtggcagcgggtctgggacagatttcactctcaccatcagcagcctgcaggctgaagatgtggcagtttattactgtcagcaattttataatactcctccgacgttcggccaagggaccaaggtggaaatcaaac

SEQ ID NO: 5 - DNA, 355 bp, Homo sapiens
  source 1..355: mol_type = other DNA
gaggtgcagctggtgcagtctggagctgaggtgaagaagcctggggcctcagtgaaggtctcctgcaaggcatctggattcaccttcagcgactactatatgcactgggttcgacaggcccctagacaagggcttgaatggatgggactaatcaatcctagtggttcgggcacagcctacgcacagaacttccagggcagagtaacgctgaccagggacacgtccacgagcacactctacatggagatgggcagcctgacatctgacgacacggccgtgtactactgtgctcgaatggactctagtgggagctatgaccactggggccagggaaccctggtcactgtctcctcag

SEQ ID NO: 6 - DNA, 331 bp, Homo sapiens
  source 1..331: mol_type = other DNA
gacatcgtgatgacccagtctccaggcaccctgtctttgtctccaggggacagagccaccctgtcctgcagggccagtcagactattcgcatcagttacttagcctggtaccagcagaagcctggccaggctcccaggctcctcgtctatggtccatccatcagggccactggcatcccagacaggttcagtgccagtgggtctgggacagacttcactctcaccatcagcagactggagcctgaagattttgcagtgtattactgtcagcattatggtagctcgcctccgaggtacacttttggccaggggaccaagctggagatcaaac

SEQ ID NO: 7 - DNA, 388 bp, Homo sapiens
  source 1..388: mol_type = other DNA
caggtgcagctggtgcagtctgggggaggcttggtccagcctggggggtccctgagactctcctgtgcagtctctggattcacctttagtagctattggatgagctgggtccgccaggctccagggaaggggctggagtgggtggccaacataaaacaagatggaagtgagaagtactatgtggactctgtgaagggccgattcaccatctccagagacaacgccaagaactcactgtttctgcaaatgaacagcctgagagccgaggacacggctgtgtattactgtgcgagagaagccgtagggtattactatgatagtcgtggttaccactactcctggtacttcgatctctggggccgtggcaccctggtcactgtctcctcag

SEQ ID NO: 8 - DNA, 331 bp, Homo sapiens
  source 1..331: mol_type = other DNA
gaaattgtgttgacccagtctccaggcaccctgtctttgtctccaggggaaagagccaccctctcctgcagggccagtcagagtgttagcagcagctacttagtctggtaccagcagaaacctggccaggctcccagactcctcatctttggtgcatccagcagggccactggcatcccagacaggttcagtggcagtgggtctgggacagacttcactctcaccatcagcagtctggagcctgaagattttgcagtgtattactgtcagcactatggtagctcacctcctatgtacacttttggccaggggaccaagctggagatcaaac

SEQ ID NO: 9 - DNA, 394 bp, Homo sapiens
  source 1..394: mol_type = other DNA
gaggtgcagctggtgcagtctggaggaggcttggtccagcctggggggtccctgagactctcctgtgcagcctctggattcacctttagtagctattggatgagctgggtccgccaggctccagggaaggggctggagtgggtggccaacataaagcaagatggaagtgagaaatactatgtggactctgtgaagggccgattcaccatctccagagacaacgccaagaactcactgtatctgcaaatgaacagcctgagagccgaggacacggctgtctattactgtgcgagagacgtaataaggacactaaattacgatattttgactggttatcattacacgcgggagttcgacccctggggccagggaaccctggtcaccgtctcctcag

SEQ ID NO: 10 - DNA, 328 bp, Homo sapiens
  source 1..328: mol_type = other DNA
caggcagggctgactcagcctgcctccgtgtctgggtctcctggacagtcgatcaccatctcccgcactggaaccagcagtgacgttggtggttataactatgtctcctggtaccaacaacacccaggcaaagcccccaaactcatgatttatgatgtcagtgatcggccctcaggggtttctaatcgcttctctggctccaagtctggcaacacggcctccctgaccatctctgggctccaggctgaggacgaggctgattattactgcagctcatatacaagcagctcttatgtcttcggaactgggaccaaggtcaccgtcctag

SEQ ID NO: 11 - DNA, 355 bp, Homo sapiens
  source 1..355: mol_type = other DNA
gaggtgcagctggtgcagtctggagctgaggtgaagaagcctgggacctcagtgaaagtttcctgcaaggcttctgggaacacagtcgccgactactatatgcactgggtacgacaggcccctagacaaggacttgagtgggtgggactaatcaatcctagtggttcgggcacagcctacgcacagaacttccaggggagagtcaccctgaccagggacacgtccacaagcacactctacatggaattgggcagccttggatctgaggacacggccatctattactgtgctcggatggactccagtgggacttatgacgtctggggccagggaaccctggtcaccgtctcctcag

SEQ ID NO: 12 - DNA, 331 bp, Homo sapiens
  source 1..331: mol_type = other DNA
gaaattgtgctgactcagtctccaggcaccctgtctttgtctccaggggaaagagccaccctctcctgcagggccagtcagagtattcgcagcggctacttagcctggtaccagcaaaaacctggccaggctcccaggctcctcatccgtggtgcgtccgtcaaggccactggcattccagacaggttcagtggcagtgggtctgggacagacttcactctcaccatcagcagactggagcctgaagattttgtagtgtattactgtcagcactttgctagctcgcctccgaggtacacttttggccaggggaccaagctggagatcaaac

SEQ ID NO: 13 - DNA, 387 bp, Homo sapiens
  source 1..387: mol_type = other DNA
caggtgcagctggtgcagtctgggggaggcttggtccagcctggggggtccctgagactctcctgtgcagcctctggattcacctttagtagttattggatgacctgggtccgccaggctccagggaaggggctggagtgggtggccaacataaagcaagatggacgtgagatatattatgtggactctgtgaagggccgattcaccctctccagagacaacgccaagaactcactttatctgcaaatgaacagcctgagagccgaggacacggctgtgttttactgtgcgagaattagagtgtttgtatcagctgactcagagggttaccactactactacggtttggacgtctggggccaggggaccacggtcaccgtctcccca

SEQ ID NO: 14 - DNA, 331 bp, Homo sapiens
  source 1..331: mol_type = other DNA
gaaacgacactcacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcaccccctacattcactttcggccctgggaccaaagtggatatcaaac

SEQ ID NO: 15 - DNA, 382 bp, Homo sapiens
  source 1..382: mol_type = other DNA
caggtgcagctggtacagtctgggggaggcgtggtccagcctgggacgtccgtgagactctcctgtgcagcctctggatttaccttcagtgaccatggcatgcactgggtccgccaggctccagggaagggcctggagtgggtggcagtcatatcctttgagggaagaaataagttctttgcagactccgtgaagggccgatccaccatctccagagacaattccaagaacacggtgtctatgcaagtggacaatgtgagacctgaggacacggctgtatattattgtgcgagagagcatttcgatggttcgggaagtgattacataggcacccgtttctttgactactggggccggggtgccctggtcaccgtctcctcgg

SEQ ID NO: 16 - DNA, 325 bp, Homo sapiens
  source 1..325: mol_type = other DNA
gacatccagttgacccagtctccatccttcctgtctgcatctgtgggagacagagtcaccatcacttgccgggccagtcagggcattagccattatttaacctggtatcagcaaaaaccagggaaagcccctaagctcctcatctatcgtacatccactttgcaaagtggggtcccgtcaaggttcagcggcagtggatctgggacagtattcactctcacaatcagcagcctgcagcctgaagatcttgcaacttattactgtcaacaccttgataatttccctacgatcaccttcggccaagggacacgactggaccttaaac

SEQ ID NO: 17 - DNA, 370 bp, Homo sapiens
  source 1..370: mol_type = other DNA
caggtgcagctggtgcagtctgggggaggcctggtcaagcctggggggtccctgagactctcctgtgcagcgtctggattcacctttaagacccataacatgatctgggtccgccaggctccagggaaggtgctggagtgggtctcgtccattagtggcagtagtagttacagatactacggagactccatgaagggccggctcaccatctccagagacaacgccaagaactcactgtatctggaaatgaacagcctgagagccgaggacacggctgtttattactgtgcgagagataagggggtgggacctactatggtctggtacttcgatctctggggccgtggcaccctggtcactgtctcctcag

SEQ ID NO: 18 - DNA, 328 bp, Homo sapiens
  source 1..328: mol_type = other DNA
gaaattgtgctgacacagtctccagccaccctgtctttgtctccaggagacagagccaccctctcgtgcagggccagtcagagtcttagtatctccctagcctggtaccaacagaaacctggccaggctcccaggctcctcatctatgatgcatccatcagggccactggcgtcccaccccggttcagtggcagtgggtctgggacagacttcactctcaccatcagcagcctagagcctgaagattttgcagtttattactgtcaacagtggagcggctggcctccgaggctcactttcggcggagggaccaaggtggagatcaaag

SEQ ID NO: 19 - DNA, 373 bp, Homo sapiens
  source 1..373: mol_type = other DNA
caggtacagctgcagcagtcaggtccaggactgatgaagccttcggagaccctgtccctcacctgcactgtctctggtggctccatcagcagtagtagttactactggggctggatccgccagcccccagggaaggggctagagtggattgggagtatctattatagtggggccacctactacaacccgtccctcaagagtcgagtcaccatatccgtagacacgtccaagaaccagctctccctgaagctgagctctgtgaccgccgcagacacggctgtatattactgtgtgaaccgatattatgattacgtttgggggagttggggggttgactgctggggccagggaaccctggtcaccgtctcgtcag

SEQ ID NO: 20 - DNA, 334 bp, Homo sapiens
  source 1..334: mol_type = other DNA
caggcagggctgactcagcctcgctcagtgtccgggtctcctggacagtcagtcaccatctcctgcactggaaccagcagtgatgtcggtggttataactatgtctcctggtaccaacagcacccaggcaaagcccccaaattgatgatttatgatgtcaataagcggccctcaggggtccctgatcgcttctctggctccaagtctggcaacacggcctccctgaccatctctgggctccaagctgaggatgaggctgattatttctgctgctcatatgcaggcagctacacgtattgggtgttcggcggagggaccaaactgaccgtcctac

SEQ ID NO: 21 - DNA, 355 bp, Homo sapiens
  source 1..355: mol_type = other DNA
caggtgcagctggtacagtctggggctgaggtgaagaagcctggggcctcaatgaaggtttcctgcaaggcatctggattcaccttcaccgactactatatgcactgggttcgacaggcccctagacaagggcttgaatggatgggactaatcaatcctagtggttcgggcacagcctacgcacagaacttccagggcagagtaacgctggccagggacacgtccacgagcacactctacatggagatgggcagcctgacatctgacgacacggccgtctactactgtgctcgaatggactctagtgggagctatgaccactggggccagggaaccctggtcactgtctcctcag

SEQ ID NO: 22 - DNA, 331 bp, Homo sapiens
  source 1..331: mol_type = other DNA
gaaacgacactcacgcagtctccaggcaccctgtctttgtctccaggggacagagccaccctgtcctgcagggccagtcagactattcgcatcagttacttagcctggtaccagcagaagcctggccaggctcccaggctcctcgtctatggtccatccatcagggccactggcatcccagacaggttcagtgcccgtgggtctgggacagacttcactctcaccatcagcagactggagcctgaagattttgcagtgtattactgtcagcattatggtagctcgcctccgaggtacacttttggccaggggaccaagctggagatcaaac

SEQ ID NO: 23 - DNA, 355 bp, Homo sapiens
  source 1..355: mol_type = other DNA
caggtgcagctggtgcagtctggggctgaggtgaagaagcctggggcctcaatgaaggtttcctgcaaggcatctggattcaccttcagcgactactatatgcactgggttcgacaggcccctagacaagggcttgaatggatgggactaatcaatcctagtggttcgggcacagcctacgcacagaacttccagggcagagtaacgctgaccagggacacgtccacgagcacactctacatggagatgggcagcctgacatctgacgacacggccgtgtactactgtgctcgaatggactctagtgggagctatgaccactggggccagggaaccctggtcactgtctcctcag

SEQ ID NO: 24 - DNA, 331 bp, Homo sapiens
  source 1..331: mol_type = other DNA
gaaattgtgctgacacagtctccaggcaccctgtctttgtctccaggggacagagccaccctgtcctgcagggccagtcagactattcgcatcagttacttagcctggtaccagcagaagcctggccaggctcccaggctcctcgtctatggtccatccatcagggccactggcatcccagacaggttcagtgccagtgggtctgggacagacttcactctcaccatcagcagactggagcctgaagattttgcagtgtattactgtcagcattatggtagctcgcctccgaggtacacttttggccaggggaccaagctggagatcaaac

SEQ ID NO: 25 - DNA, 355 bp, Homo sapiens
  source 1..355: mol_type = other DNA
gaggtgcagctggtgcagtctggagctgaggtgaagaagcctggggcctcagtgaaattttcctgcaaggcttctggattcagagtcgacggctcctatatgcactgggtacgacaggcccctagaggaggacttgagtggatgggactaatcaaccctagtggttcgggcacagcctacgcacagatcttccagggcagagtaaccctgacaagggacacgtctacgagcacactctacatggaactgggcagtctggggtctgaagacacggccatgtattattgtgctcgaatggaccgcagtgggagctatgacctctggggccagggaaccctggtcatcgtctcctcag

SEQ ID NO: 26 - DNA, 331 bp, Homo sapiens
  source 1..331: mol_type = other DNA
gacattgtgctgacccagtctccaggcaacctgtctttgtccccaggggaaagagccaccctctcctgcagggccagtgagagtattcgcagcgcctacatagcctggtaccagcagaaacctgggcaggctcccaggctcctcatgcgtggtgcatctgtcagggccgctggcattccagacaggttcagtggcagtgggtctgggacagactacgctctcaccatcagcaggctggagcctgaagattttgcagtgtactactgtcagcactttgctagctcgcctccgaggtacacttttggccaggggaccaagctggagatcaaac

SEQ ID NO: 27 - protein, 123 aa, Homo sapiens
QVQLQQSGPEVKKPGTSVKVSCRASGFTFSSSAMQWVRQARGQRLEWIGWIVVGSGNANYAQKFQERVTITRDLSTRTAYMELSSLRSEDTAVYYCAAPYCSGGTCLDGFDIWGQGTMVTVSS

SEQ ID NO: 28 - protein, 108 aa, Homo sapiens
EIVLTQSPGTLSLSPGERATLSCRASQSVSRNYLGWYQQKPGQAPRLLIYGASSRATGIPDRFSGSGSGTDFTLTISRLEPEDFAVYYCQQYGSSPWTFGQGTKVEIK

SEQ ID NO: 29 - protein, 124 aa, Homo sapiens
QVQLVQSGAEVKKPGASVKVSCKASGYIFTDYFIHWVRQAPGQGLEWMGWINPNSGGTKYAQKFQGRVTMTRDTSISTVYMELSRLRSDDTAVHYCTRDLPLRTLYDSYGFDSWGQGTLVTVSS

SEQ ID NO: 30 - protein, 110 aa, Homo sapiens
LPVLTQPASVSGSPGQSITISCTGTSSDVGSYNLVSWYQQHPGKAPKLMIYDVSKRPSGVSNRFSGSKSGNTASLTISGLQAEDEADYYCCSYAGNSIVFFGGGTKLTVL

(19) United States
(12) Patent Application Publication
(10) Pub. No.: US 2024/0270828 A1
(43) Pub. Date: Aug. 15, 2024

(54) HUMAN NEUTRALIZING ANTIBODIES AGAINST SARS-COV-2 SPIKE S2 DOMAIN AND USES THEREOF

(71) Applicants: THE UAB RESEARCH FOUNDATION, Birmingham, AL (US); TEXAS BIOMEDICAL RESEARCH INSTITUTE, San Antonio, TX (US)

(72) Inventors: James KOBIE, Birmingham, AL (US); Michael PIEPENBRINK, Birmingham, AL (US); Paul GOEPFERT, Birmingham, AL (US); Mark R. WALTER, Vestavia Hills, AL (US); Nathan ERDMANN, Birmingham, AL (US); Ashlesha DESHPANDE, Vestavia Hills, AL (US); Luis MARTINEZ-SOBRIDO, San Antonio, TX (US); Jun-Gyu PARK, San Antonio, TX (US)

(21) Appl. No.: 18/688,935

(22) PCT Filed: Sep. 6, 2022
(86) PCT No.: PCT/US2022/075991

§ 371 Date: Mar. 4, 2024

Related U.S. Application Data
(60) Provisional application No. 63/240,655, filed on Sep. 3, 2021.

(51) Int. Cl. C07K 16/10; A61K 39/00; A61P 31/14

(52) U.S. Cl. CPC C07K 16/1003; A61P 31/14; A61K 2039/505; C07K 2317/24; C07K 2317/73; C07K 2317/76; C07K 2317/92

(57) ABSTRACT

The disclosure is based, at least in part, on certain human monoclonal antibodies, or antigen binding fragments thereof, having unexpected broad neutralizing activities against SARS-CoV-2. The disclosed antibodies and/or antigen-binding fragments thereof are therapeutic agents for the treatment of SARS-CoV-2 infections and are suitable for use in therapeutic methods to protect individuals from SARS-CoV-2 infections.

### BACKGROUND

Coronaviruses (CoVs) are members of the family Coronaviridae. The Coronaviridae has 4 separate genera, the Alphacoronavirus, Betacoronavirus, Deltacoronavirus, and Gammacoronavirus with each genus having one or more subgenus. The Alphacoronaviruses and Betacoronaviruses mainly infect bats, but they also infect other species such as humans, camels, rabbits, dogs and masked palm civets. CoVs are enveloped viruses that possess extraordinarily large single-stranded RNA genomes ranging from 26 to 32 kilobases in length. CoVs were historically regarded as pathogens that only cause mild diseases. Currently, at least seven CoV species are known to cause diseases in humans. HCoV-229E, HCoV-OC43, HCoV-NL63 and HCoV-HKU1 generally cause only mild common cold symptoms. Severe illness can be caused by the remaining three viruses, each in the Betacoronavirus genus. SARS-CoV resulted in the outbreak of sever acute respiratory syndrome (SARS) in 2002 and 2003. MERS-CoV was responsible for Middle East respiratory syndrome (MERS), which emerged in 2012 and remains in circulation in camels. A small outbreak of MERS-CoV was reported on Jun. 2, 2020 in Saudi Arabia (9 case reported with 5 deaths). Finally, SARS-CoV-2, which emerged in December 2019 in Wuhan province of China, and causes COVID-19. The COVID-19 pandemic is impacting virtually every country around the world, with no immediate prospects for easing. The US is currently topping the world in the number of infected people with more cases in sight.

The fatality rate for SARS was approximately 10%, with the risk for a fatal outcome increasing with age, while the mortality of SARS-CoV-2 infection is estimated to be around 1.5 to 10%, with the risk for fatal outcome increasing with age and certain comorbidities. The fatality rate for MERS-CoV was approximately 37%. In addition, SARS-CoV-2 has a basic reproduction rate (Ro) of 3.3-5.5, which is higher than those of SARS-CoV and MERS-CoV (2.7-3.9), indicating a higher transmissibility of SARS-CoV-2 than other human coronaviruses. Unlike SARS and MERS which each infected less than 10,000 individuals, COVID 19 has currently infected more than 16,000,000 individuals and has been responsible for over 650,000 deaths worldwide.

Despite the availability of vaccines, viral evolution is driving the emergence and dominance of waves of SARS-CoV-2 variants, which have mutations in the spike (S), which reduces vaccine efficacy. Thus far therapeutic monoclonal antibodies (mAbs) against SARS-CoV-2 have targeted the receptor binding domain (RBD) of the spike, however variants have emerged for which these mAbs have decreased potency. As such, there remains a need for improved compositions and methods for the treatment of SARS-CoV-2.

### BRIEF DESCRIPTION OF THE FIGURES

FIGS. 1A and 1B show: a depiction of the S2 domains of SARS-CoV-1 spike, SARS-CoV-2 spike, and a SARS-COV-1/2 chimera (FIG. 1A); and binding of various mAbs to SARS-CoV-2 spike or SARS-CoV-1/2 chimera spike (FIG. 1B) as measured by ELISA.

FIG. 2 shows flow cytometric plots of human B lymphocytes binding to SARS-CoV-2 spike and/or SARS-CoV-1/2 chimera spike.

FIG. 3 depicts the binding of mAbs to SARS-CoV-2 S2-STBL, SARS-CoV-1/2 S1S2 chimera, SARS-CoV-2 S2, SARS-CoV-2 S1S2 or SARS-CoV-2 Si (negative control) as measured by the area under the curve of the optical density in an ELISA.

FIGS. 4A and 4B show representative wells (FIG. 4A) and mAb dose plotted against percent viral (SARS-CoV-2 WT S2) infection for a live virus neutralization antibody assay involving treatment with 1249A8 mAb (FIG. 4B). Mean and standard error are depicted.

FIGS. 5A and 5B show the structure of 1249A8/MERS-CoV SHp complex and related SH-targeting NMAbs. FIG. 5A shows a ribbon diagram of the 1249A8/MERS-CoV SHp complex and other human NMAb/SHp complexes. 1249A8 heavy and light chains are shaded light gray and gray, respectively. The MERS-CoV SHp has a gradient of shading from the N-terminus (top) to the C-terminus (bottom). Other NAb-SHp structures are shaded as described for 1249A8. Labels for each NMAb-SH complex contain the designation of C1 (class-1) or C2 (class-2) to distinguish their epitopes. The linear SH epitope for each NMAb is shown on the MERS-CoV and SARS-CoV-2 sequences as a shaded bar. Conserved residues in the linear sequence are bolded. The invariant core-SH region is highlighted by the black bar. FIG. 5B depicts NMAb binding epitopes superimposed on a model of the SARS-CoV-2 pre-fusion S trimer (6xr8) and the extended C-terminal helix residues 1,171-1,203 (pdbid 61vn). NMAbs are shaded as shown in FIG. 5A.

FIGS. 6A-6D show NMAb binding to SH peptides and SH within CoV S proteins. Fractional binding of SH peptide (FIG. 6A) and CoV S proteins: SARS-CoV-2-2P omega B.1.1.529 S (FIG. 6B) MERS-CoV S (FIG. 6C) and SARS-CoV-2-6P omega B.1.1.529 S (FIG. 6D).

FIGS. 7A-7D show details of the 1249A8/MERS-CoV SHp epitope. FIG. 7A shows surface representation of 1249A8 with heavy and light chains shaded as in FIG. 5, with the MERS-CoV SHp shown in the foreground. Optimized views of the MERS-CoV SHp interacting with the 1249A8 heavy (FIG. 7B) and light (FIG. 7C) chains. FIG. 7D shows the superposition of SARS-CoV-2 SHp (7rnj) onto the MERS-CoV SHp. The superimposed SHps are shown with the 1249A8 Fab surface showing that both SHps fit in the 1249A8 binding pocket without steric clashes.

FIGS. 8A-8D show SARS-CoV-2 pre- and post-fusion structures and mimicry by 1249A8.

FIG. 8A shows the location of the SHps in the structures of the SARS-CoV-2 pre-fusion (6rx8+61vn) and post-fusion (6xra) S structures. The location of 6HB1 and 6HB2 are shown on the post-fusion S. FIG. 8B shows the packing of the SHp with the 3-helix region against the CH region. FIG. 8C shows 1249A8 mimics the SARS-CoV-2 S 3-helix region loop (residues 743-749), which caps the N-terminal end of the post-SH core helix. FIG. 8D shows distinct SH binding epitopes that target the N-terminal of end of SH (1249A8) and the C-terminal end of SH (CV3-25).

FIGS. 9A and 9B show 1249A8 locks SH in a pre-fusion α-helical conformation. FIG. 9A shows a ribbon diagram with pre- and post-fusion SH regions (residues 1139-1162) from pdbids 6xr8 and 6xra superimposed onto the 1249A8 binding epitope (SH residues 1147-1159, red). Boxed residues highlight the residues mimicking the post-fusion core α-helix (1147-1154) and the end of the epitope (1159). FIG. 9B shows superposition of the structure of FIG. 9A in the context of the 1249A8 Nab surface, which shows 1249A8 cannot bind to the full length pre-fusion α-helix and how 1249A8 disrupts the secondary structure of the post-fusion SH, locking it into the pre-fusion conformation.

FIG. 10 is a chart showing the burying of MERS-SH and CoV-2 SH residues in 1249A8, S2P6, CC40.8 and CV3-25.

FIGS. 11A-11D show isolation of SARS-CoV-2 S2-specific human monoclonal antibodies (hmAbs). FIG. 11A shows a schematic representation of the S2-STBL and S1/S2 chimera proteins used as baits for ELISA and flow cytometry. FIG. 11B shows human plasma from either convalescent or healthy subjects was diluted 1:1000 in PBS and tested in duplicate in an ELISA against indicated proteins; Absorbance at 450 nM is shown. Each row is an individual subject. FIG. 11C shows a representative gating strategy for S2+ B cell isolation. Initial plots are gated on live CD3-CD4-CD14-annexinV-CD19+CD27+ B cells. FIG. 11D shows hmAbs tested at 10 and 1 μg/ml in duplicate by ELISA for binding to indicated protein; area under the curve (AUC) is indicated.

FIGS. 12A-12E show in vitro neutralization and ADCP of SARS-CoV-2 by S2-specific hmAbs. FIG. 12A shows SARS-CoV-2 neutralization of S2 hmAbs. Vero E6 cells were infected with SARS-CoV-2 WA-1 or SARS-CoV-2 Delta for 1 h. After 1 h of viral adsorption, the indicated concentrations of S2 hmAbs were added and at 24 h.p.i., infected cells were fixed for virus titration by immunostaining assay. Data was expressed as mean and SD of quadruplicates. FIG. 12B shows a summary of viral neutralization (NT₅₀) using either pseudovirus representing SARS-CoV-2 D614G mutation, or live SARS-CoV-2 WA1 or Delta. FIG. 12C shows Ab-dependent cellular phagocytosis (ADCP) assay. SARS-CoV-2 Wuhan-Hu-1 S-coated and BSA coated beads were incubated with 5 μg/ml hmAb for 2 h and then added to THP-1 cells. After incubation for 3 h at 37° C., cells were assayed for fluorescent bead uptake by flow cytometry. The ADCP score of each mAb was calculated by multiplying the percentage of bead positive cells (frequency of phagocytosis) by the mean fluorescence intensity (MFI) of the beads (degree of phagocytosis) and dividing by 10⁶. FIG. 12D shows binding to S2 protein fragments by hmAbs (5 μg/ml) determined by ELISA. FIG. 12E shows SPR competition assays performed by capturing S2-Frag4 to the chip surface, followed by sequential injections of 50 nM of 1249A8 (Inject1) and the various S2 Abs at 50 nM concentration (Inject 2). (Inset) Summary of the competition sensorgram data, where Ab binding levels (RU), measured after Inject1 (black) were normalized to 100, and compared to Ab binding levels after the second injection (Inject 2), which occurred after 1249A8 binding.

FIGS. 13A-13J show prophylactic activity of 1249A8 hmAb against rSARS-CoV-2 WA1-Venus and rSARS-CoV-2 Beta-mCherry in K18 ACE2 transgenic mice model. Female K18 hACE2 transgenic mice were treated i.p. with 1249A8 (10 mg/kg or 40 mg/kg), 1213H7 (5 mg/kg), alone or in combination, or isotype control hmAb (40 mg/kg), followed by infection with both rSARS-CoV-2 Venus and rSARS-CoV-2 Beta/mCherry Beta. Body weight (FIG. 13A) and survival (FIG. 13B) were evaluated at the indicated days post-infection (n=5 mice/group). Mice that loss >25% of their body weight were humanely euthanized. Error bars represent standard deviations (SEM) of the mean for each group of mice. Viral titers in the nasal turbinate (FIG. 13C) and lung (FIG. 13D) at 2 and 4 DPI were determined by plaque assay in Vero E6 cells (n=4 mice/group/day). Symbols represent individual mice, bars indicate the mean and SD of lung virus titers. Dotted lines indicate limit of detection, titers below limit of detection are presented at limit of detection. Proportion of rSARS-CoV-2 WA-1/Venus and rSARS-CoV-2 Beta/mCherry determined by fluorescence in nasal turbinate (FIG. 13E) and lung (FIG. 13F). At 2 and 4 DPI, lungs were collected to determine Venus and mCherry fluorescence expression using an Ami HT imaging system FIG. 13G. BF, bright field. Venus (FIG. 13H) and mCherry (FIG. 13I) radiance values were quantified based on the mean values for the regions of interest in mouse lungs. Mean values were normalized to the autofluorescence in mock-infected mice at each time point and were used to calculate fold induction. FIG. 13J shows gross pathological scores in the lungs of mock-infected and rSARS-CoV-2-infected K18 hACE2 transgenic mice were calculated based on the percentage of area of the lungs affected by infection. Dotted line indicates limit of detection.*indicates p<0.05 as compared to isotype control hmAb as determined by one-way ANOVA.

FIGS. 14A-14E show universal 0-coronavirus invitroactivity of 1249A8 hmAb. FIG. 14A shows confluent monolayers of Vero E6 cells were infected (MOI 0.1) with SARS-CoV-2 WA-1, Beta (B.1.351), Gamma (P.1), or Epsilon (B.1.427/B.1.429). Mock-infected cells (bottom) were included as control. In a separate experiment, confluent monolayers of Vero E6 cells were infected (MOI 0.1) with SARS-CoV (Urbani v2163) or MERS-CoV (recombinant MERS—CoV-RFP delta ORF5 ic). Cells were incubated with the indicated primary S2 hmAb (1 μg/ml) and developed with a FITC-conjugated secondary anti-human Ab. 4,6-Diamidino-2-phenylindole (DAPI) was used for nuclear stain. As internal control, mock and SARS-CoV-2 WA-1 infected cells were stained with a SARS-CoV cross-reactive NP mAb (1C7C7) or a-MERS NP pAb. Scale bars indicate 50 m. FIG. 14B shows binding of hmAb 1249A8 and other known S2-specific mAbs at 5, 0.5, and 0.05 μg/ml to the Spike proteins of β-coronaviruses as determined by ELISA in the presence or absence of 8M urea. FIG. 14C shows a summary table of Surface Plasmon Resonance (SPR) and Biolayer Interferometry (BLI) of 1249A8 against the Spike protein of SARS-CoV-2, MERS-CoV, and SARS-CoV. FIG. 14D shows viral neutralization by 1249A8 and CV3-25. Vero E6 cells were infected with 100 PFU of SARS-CoV (Urbani v2163) or MERS-CoV (recombinant MERS—CoV-RFP delta ORF5 ic) for 1 h. After 1 h of viral adsorption, the indicated concentrations of S2 hmAbs were added. At 24 h.p.i., infected cells were fixed for virus titration by immunostaining assay. FIG. 14E shows ADCP of S2-specific mAb against MERS-CoV Spike coated beads.

FIGS. 15A-15E show neutralization and prophylactic in vivo activity of 1249A8 and 1213H7 against SARS-CoV-2 Omicron. FIG. 15A shows binding of 1249A8 (60, 40, 27, 18, 12 nM) to SARS-CoV-2 Wuhan and Omicron Spike protein determined by BLI. FIG. 15B shows vero AT cells were infected with 600 pfu SARS-CoV-2 Omicron (BEIR) and after 1 h of viral adsorption, the indicated mAb(s) was added and at 24 h.p.i infected cells were fixed for virus titration by immunostaining assay. 1213H7 and 1249A8 were tested alone (open symbols) and together keeping 1213H7 constant (C) (50 ng/ml) or 1249A8 constant (2 μg/ml) and titrating the reciprocal mAb (closed symbols). Resulting NT₅₀(ng/ml) are indicated. K18 hACE2 mice were treated with 1249A8 (40 mg/kg), 1213H7 (10 mg/kg), or isotype control mAb (40 mg/kg) either alone or in combination i.p. or i.n. as indicated and 24 h later challenged i.n. with 10⁵ PFU SARS-CoV-2 Omicron (BEIR) and virus titer in nasal turbinates (FIG. 15C) and lungs (FIG. 15D) determined at 2 and 4 dpi by plaque assay and gross lung pathology measured (FIG. 15E) (n=4 mice/group/day). Each symbol represents an individual animal. Dotted line indicates limit of detection, titers below limit of detection are presented at limit of detection.*indicates p<0.05 compared to isotype control group as determined by one-way ANOVA.

FIGS. 16A-16D show therapeutic activity of intranasal 1249A8 and 1213H7 in hamsters infected with SARS-CoV-2 Delta. Golden Syrian hamsters were infected i.n. with 10⁴ CCID50SARS-CoV-2 Delta and 12 h p.i. treated i.n. with a single dose of indicated mAb(s). n=4-8 per group. Body weight was measured daily (FIG. 16A). Mean±SEM indicated. Nasal turbinate (FIG. 16B), cranial lung (FIG. 16C), and caudal lung (FIG. 16D) viral titers were measured at 3 d p.i. by plaque assay. Each symbol represents an individual animal. Dotted line indicates limit of detection, titers below limit of detection are presented at limit of detection.*indicates p<0.05 compared to isotype control group as determined by one-way ANOVA.

FIGS. 17A-17E show therapeutic activity of intranasal 1249A8 and 1213H7 in hamsters infected with SARS-CoV. Golden Syrian hamsters were infected i.n. with 10⁴ pfu SARS-CoV (SARS-Urbani) and 12 h p.i. treated i.n. with a single dose of indicated mAb(s). n=4-8 per group. Body weight was measured daily (FIG. 17A). Mean±SEM indicated. Oropharyngeal swabs were collected days 1, 2, and 3 p.i. and sum of daily virus titer for each animal indicated (FIG. 17B). Nasal turbinate (FIG. 17C), cranial lung (FIG. 17D), and caudal lung (FIG. 17E) viral titers were measured at 3 d p.i. by plaque assay. Each symbol represents an individual animal.*indicates p<0.05 compared to isotype control group as determined by one-way ANOVA.

FIG. 18 shows vero E6 cells infected with SARS-CoV-2 WA-1 (FIG. 18A) or SARS-CoV-2 Delta (FIG. 18B), and 1 hour after viral adsorption mAb was added at indicated concentrations in triplicate. At 24 h p.i. cells were fixed, stained with anti-NP mAb 1C7C7 and quantified using ELISPOT.

FIG. 19 shows a comparison of pre- and post-mAb treatment in neutralization assay. For pre-treatment (top), 100 PFU/well of SARS-CoV-2 WA-1 containing indicated concentrations of mAbs were mixed and incubated for 1h. Vero HL cells were infected with virus-mAb mixture as virus adsorption for 1h, followed by changing media. For post-treatment (bottom), Vero HL cells were infected with 100 PFU/well of SARS-CoV-2 WA-1. After 1 h of viral adsorption, the media was changed with indicated concentrations of mAb. At 24 h p.i., infected cells were fixed, immunostained using anti-NP mAb 1C7C7, and quantified using ELISPOT.

FIG. 20 shows analysis of 1249A8 binding to CoV Spikes, wherein 1249A8 was tested at indicated concentrations by ELISA for binding to indicated Spike proteins.

FIGS. 21A and 21B show surface plasmon resonance binding. Using a Biacore T200, mAbs were immobilized and the binding kinetics for the interaction between mAbs and Spike protein was determined by injecting four concentrations of SARS-CoV-2 S1S2 (FIG. 21A) or MERS-CoV S2 (FIG. 21).

FIG. 22 shows neutralization of SARS-CoV-2 WA-1 by combined 1213H7 and 1249A8. Vero AT cells were infected with SARS-CoV-2 WA-1 and after 1 h of viral adsorption, the indicated mAb(s) was added and at 24 h.p.i infected cells were fixed for virus titration by immunostaining assay. 1213H7 and 1249A8 were tested alone (open symbols) and together keeping 1213H7 constant (C) (50 ng/ml) or 1249A8 constant (2 μg/ml) and titrating the reciprocal mAb (closed symbols). Resulting NT₅₀ (ng/ml) are indicated.

### DETAILED DESCRIPTION

The disclosure provides for broadly neutralizing anti-SARS-CoV-2 monoclonal antibodies and antigen-binding fragments thereof. The disclosure further provides for pharmaceutical compositions comprising an antibody of the disclosure and methods of using the antibodies of the disclosure. The antibodies of the disclosure are shown to be effective in treating a SARS-CoV-2 infection. The antibodies of the disclosure bind epitopes within the spike protein of SARS-CoV-2. In certain embodiments, the antibodies of the disclosure bind an epitope within the receptor binding domain (RBD) of the SARS-CoV-2 spike protein. In certain embodiments, the antibodies of the disclosure bind an epitope within the receptor binding moiety (RBM) of the SARS-CoV-2 spike protein. In certain embodiments, the antibodies of the disclosure bind an epitope within the S2 region of the SARS-CoV-2 spike protein.

The S2 domain of SARS-CoV-2 spike is responsible for viral fusion with host cells, hence S2 is critically involved in infection. S2 is highly conserved among different clinical isolates of the SARS-CoV-2 (and SARS), and shares homology with endemic CoV. Applicant assessed whether mAbs against this region of S conferred protection against a broad spectrum of clinical isolates.

As discussed in more detail herein, the antibodies of the disclosure were isolated from a subject with a documented SARS-CoV-2 infection, wherein the subject subsequently recovered from the SARS-CoV-2 infection. As such, the subject mounted an effective immunological response to the SARS-CoV-2 infection. Using single-cell immunoglobulin cloning, neutralizing antibodies to SARS-CoV-2 were isolated from the subject.

### Definitions

All patent applications, patents, and printed publications cited herein are incorporated herein by reference in the entireties, except for any definitions, subject matter disclaimers or disavowals, and except to the extent that the incorporated material is inconsistent with the express disclosure herein, in which case the language in this disclosure controls.

As used herein, the term “about” refers to within 10%, preferably within 5%, and more preferably within 1% of a given value or range. Alternatively, the term “about” refers to within an acceptable standard error of the mean, when considered by one of ordinary skill in the art.

As used herein, the term “affinity” refers to the strength of the sum total of noncovalent interactions between a single binding site of an antibody or other molecule and its binding partner (such as, but not limited to, an antigen). Unless indicated otherwise, the term “binding affinity” refers to intrinsic binding affinity which reflects a 1:1 interaction between an antibody and an antigen or between members of a binding pair. Affinity is generally be represented by the dissociation constant (KD).

As used herein, the term “antibody” includes whole antibodies and any antigen binding fragment thereof. Examples of an antibody include, but are not limited to, monoclonal antibodies, polyclonal antibodies, multispecific antibodies (for example, bispecific antibodies) formed from at least two antibodies or antigen binding fragments thereof, chimeric antibodies, anti-idiotypic (anti-Id) antibodies, intrabodies, and antigen binding fragments of any of the foregoing, Whole antibodies are glycoproteins comprising at least two heavy (H) chains and two light (L) chains inter-connected by disulfide bonds. Each heavy chain is comprised of a heavy chain variable (V_{H}) region and a heavy chain constant (C_{H}) region. The C_{H} region is comprised of three to four domains, C_{H1}, C_{H2}, C_{H3}, and C_{H4}. Each light chain is comprised of a light chain variable (V_{L}) region and a light chain constant region. The light chain constant region is comprised of one domain, C_{L}. The V_{H} and V_{L} regions can be further subdivided into regions of hypervariability, termed complementarity determining regions (CDRs), interspersed with regions that are more conserved, termed framework regions (FRs). Each V_{H} and V_{L} region is composed of three CDRs and four FRs, arranged from amino-terminus to carboxy-terminus in the following order: FR1, CDR1, FR2, CDR2, FR3, CDR3, and FR4. The V_{H} and V_{L} regions form a binding domain that interacts with an antigen in an antigen-specific manner. The C_{H} and C_{L} regions mediate binding of the antibody to host tissues, cells or factors, including various cells of the immune system (e.g., effector cells) and the first component (CIq) of the classical complement system. Antibodies may be of any type, including IgG, IgE, IgM, IgD, IgA and IgY and of any class, including, class IgG1, IgG2, IgG3, IgG4, IgA1 and IgA2 or subclass.

As used herein, the terms “antigen-binding fragment” or “antigen-binding portion” refer to one or more fragments derived from an antibody described herein (a parent antibody) that retain the ability to specifically bind to the same antigen as the parent antibody. Examples of binding fragments include, but are not limited to, a Fab fragment (a monovalent fragment consisting of the V_{L}, V_{H}, CL and C_{H1} domains), a F(ab)2 fragment (a bivalent fragment comprising two Fab fragments linked by a disulfide bridge at the hinge region), a Fab′ fragment (an Fab fragment comprising a portion of the hinge region), a F(ab′)2 fragment (a bivalent fragment comprising two Fab fragments linked by a disulfide bridge at the hinge region and containing a portion of the hinge region) a Fd fragment (a monovalent fragment consisting of the V_{H} and C_{H1} domains), a Fv fragment (a monovalent fragment consisting of the V_{L} and V_{H} domains of a single arm of an antibody), a disulfide-linked Fv (sdFv), a dAb fragment (a monomeric fragment consisting of a V_{H} domain), an isolated CDR, a nanobody or single domain antibody (a monomeric fragment consisting of a single variable antibody domain), a portion of the V_{H} region containing a single CDR and two FRs, a portion of the V_{L} region containing a single CDR and two FRs, a diabody (an antibody fragments with two antigen-binding sites; such diabody may be bivalent or bispecific), a triabody (an antibody fragments with three antigen-binding sites; such triabody may be trivalent or trispecific), and a tetrabody (an antibody fragments with four antigen-binding sites; such tetrabody may be tetravalent or tetraspecific). The terms also include single chain Fv (scFv) which are created by recombinantly joining the V_{H} and V_{L} genes by a synthetic linker and expressed as a single polypeptide. Examples of scFv include, but are not limited to, scFv-FC, scFv-CH, scFab, and scFv-zipper. An antigen-binding fragment as described herein may be obtained using conventional methods known in the art and tested for binding as is done with conventional whole antibodies. Suitable antigen-binding fragments are described in Pluckthun (The Pharmacology of Monoclonal Antibodies, vol. 113, Rosenburg and Moore eds., (Springer-Verlag, New York), pp. 269-315 (1994)), Hudson et al., Nat. Med. 9:129-134 (2003), WO 93/16185, U.S. Pat. Nos. 5,571,894 and 5,587,458.

As used herein, the term “antibody of the disclosure” means an antibody disclosed herein, and includes pharmaceutically acceptable forms thereof, such as, but not limited to, pharmaceutically acceptable salts, hydrates and/or solvates. In certain embodiments, an antibody of the disclosure is an antigen-binding fragment.

As used herein, the term “antibody variant” refers to any modified form of an antibody described herein, such as, but not limited to, an antibody having one or more substitutions, deletions or insertions relative to a parental antibody and an antibody linked to a protein or non-protein moiety.

As used herein, the terms “binds to an epitope” or “recognizes an epitope” with reference to an antibody refers to the epitope bound by the antibody. The term does not require the antibody to directly contact every amino acid within the epitope.

As used herein, the term “binds to the same epitope” with reference to two or more antibodies means that the antibodies bind to the same or overlapping amino acids, whether such amino acids are continuous or discontinuous segments. The term does not require the antibodies bind to or contact exactly the same amino acids. The precise amino acids which the antibodies contact can differ. In one example, a first antibody can bind to a group of amino acids that is completely encompassed by the group of amino acids bound by a second antibody. In another example, a first antibody can bind to a group of amino acids that overlap with a group of amino acids bound by a second antibody.

As used herein, the term “chimeric antibody” refers to an antibody in which at least a portion of the variable region sequences (including CDR and FR sequences or just CDR sequences) are derived from one species (for example, a rat) and the constant region sequences are derived from another species (for example, a human). The term also includes an antibody in which its variable region sequence or CDR(s) is derived from one source (e.g., an IgA1 antibody) and the constant region sequence or Fc is derived from a different source (e.g., a different antibody, such as an IgG, IgA2, IgD, IgE or IgM antibody). Chimeric antibodies are described in U.S. Pat. No. 4,816,567 and Morrison et al., Proc. Natl. Acad. Sci. USA, 81:6851-6855 (1984)).

As used herein, the term “detectable label” refers to a molecule capable of being detected in a subject or an assay, including, but not limited to, radioactive isotopes, fluorescent compounds, chemiluminescent compounds, chromophores, enzymes, enzyme substrates, enzyme cofactors, enzyme inhibitors, chromophores, dyes, metal ions, metal sols, ligands, intercalating dyes and the like.

As used herein, the term “epitope” refers to an antigenic determinant that interacts with (is bound by) a specific antigen binding site in the variable region of an antibody molecule (the paratope). A single antigen (such as, but not limited to, a polypeptide) may have more than one epitope. Thus, different antibodies may bind to different epitopes on an antigen and may have different biological effects depending on which epitope is bound. The term “epitope” also refers to a site on an antigen to which B and/or T cells respond. It also refers to a region of an antigen that is bound by an antibody. Epitopes may be defined as a structural epitope (the portion of the antigenic determinant that is contacted by the CDR loops of an antibody) or a functional epitope (a subset of a structural epitope comprising those energetic residues centrally located in the structural epitope and directly contribute to the affinity of the antibody-epitope interaction). Epitopes may become immunologically available after fragmentation or denaturation of an antigen (a cryptotope). Epitopes may be linear or conformational (composed of non-linear amino acids brought together in a folded three-dimensional structure). Epitopes may include residues that are chemically active surface groupings of molecules such as amino acids, sugar side chains, phosphoryl groups, or sulfonyl groups, and may have specific three-dimensional structural characteristics, and/or specific charge characteristics. An epitope typically includes at least 3 to 15 amino acids.

As used herein, the term “epitope mapping” refers to the process of identification of an epitope for antibody-antigen recognition.

As used herein, the term “functional equivalent” with reference to an antibody disclosed herein refers to an antibody variant of a parent antibody that retains one or more characteristics (such as, but not limited to, binding to the same epitope) of the parent antibody as disclosed herein. A functional equivalent may optionally differ in one or more characteristics of the parent antibody (such as, but not limited to, binding affinity, ADCC, and/or CDC).

As used herein, the term “human antibody” refers to antibodies having variable regions in which both the framework and CDR regions are derived from human germline immunoglobulin sequences. Furthermore, if the antibody contains a constant region, the constant region also is derived from human germline immunoglobulin sequences. Human antibodies can include amino acid residues not encoded by human germline immunoglobulin sequences (such as mutations introduced by random or site-specific mutagenesis in vitro or by somatic mutation in vivo). As used herein, the term human antibody is not intended to include antibodies in which CDR sequences are derived from the germline of another mammalian species, such as a mouse, that have been grafted onto human framework sequence and/or constant regions.

As used herein, the term “human monoclonal antibody” refers to a monoclonal antibody that is obtained from a human.

As used herein, the term “immune response” refers to a biological response within a vertebrate against a foreign agent, which response protects the vertebrate, at least partially, against the foreign agent and diseases caused by the foreign agent. An immune response is mediated by the action of a cell of the immune system (for example, a T lymphocyte, B lymphocyte, natural killer (NK) cell, macrophage, eosinophil, mast cell, dendritic cell or neutrophil) and soluble macromolecules produced by any of these cells or the liver (including antibodies, cytokines, and complement) that results in selective targeting, binding to, damage to, destruction of, and/or elimination from the vertebrate's body of invading foreign agent, cells or tissues infected with the foreign agent. In preferred embodiments, as used herein, the term “immune response” does not include a response to a self-antigen.

As used herein, the term “isolated antibody” refer to an antibody that is substantially free of other antibodies having different antigenic specificities. An isolated antibody can optionally be substantially further free of other cellular material and/or reagents.

As used herein, the term “isotype” refers to the antibody class (IgG, including IgG1-IgG4, IgM, and IgA. Including IgA1 and IgA2, IgD and IgE) that is encoded by the heavy chain constant region genes.

As used herein, the term “monoclonal antibody” refers to antibody molecules of a single molecular composition such that each of the antibody molecules displays a single binding specificity and binding affinity for a given epitope.

As used herein, the term “pharmaceutically acceptable” refers to a compound that is compatible with an antibody of the disclosure or other ingredients of a composition and not deleterious to the subject receiving the antibody of the disclosure or composition. In some embodiments, the term “pharmaceutically acceptable” means approved by a regulatory agency of the Federal or a state government or listed in the U.S. Pharmacopeia or other generally recognized pharmacopeia for use in animals, and more particularly in humans.

As used herein, the term “pharmaceutically acceptable carrier or excipient” refers to a carrier medium or an excipient which does not interfere with the effectiveness of an antibody of the disclosure or other active ingredient of the composition and which is not toxic to the subject at the concentrations at which it is administered. The term includes, but is not limited to, a solvent, a stabilizer, a solubilizer, a tonicity enhancing agent, a structure-forming agent, a suspending agent, a dispersing agent, a chelating agent, an emulsifying agent, an anti-foaming agent, an ointment base, an emollient, a skin protecting agent, a gel-forming agent, a thickening agent, a pH adjusting agent, a preservative, a penetration enhancer, a complexing agent, a lubricant, a demulcent, a viscosity enhancer, a bio-adhesive polymer, or a combination thereof. The use of such agents for the formulation of pharmaceutically active substances is well known in the art (see, for example, “Remington's Pharmaceutical Sciences”, E. W. Martin, 18^{th} Ed., 1990, Mack Publishing Co.: Easton, Pa.).

As used herein, the term “pharmaceutically acceptable salt” refers to salts derived from inorganic or organic acids including, for example hydrochloric, hydrobromic, sulfuric, nitric, perchloric, phosphoric, formic, acetic, lactic, maleic, fumaric, succinic, tartaric, glycolic, salicylic, citric, methanesulfonic, benzenesulfonic, benzoic, malonic, trifluoroacetic, trichloroacetic, naphthalene-2 sulfonic and other acids. Pharmaceutically acceptable salt forms may also include forms wherein the ratio of molecules comprising the salt is not 1:1. For example, the salt may comprise more than one inorganic or organic acid molecule per molecule of antibody, such as two hydrochloric acid molecules per molecule of antibody. As another example, the salt may comprise less than one inorganic or organic acid molecule per molecule of antibody, such as two molecules of compound of antibody per molecule of tartaric acid. Salts may also exist as solvates or hydrates.

A “pharmaceutical composition” refers to a mixture of one or more of the antibodies of the disclosure, with other components, such as, but not limited to, pharmaceutically acceptable carriers and/or excipients. The purpose of a pharmaceutical composition is to facilitate administration of a compound of disclosure.

As used herein, the term “recombinant antibody” includes all antibodies that are prepared, expressed, created or isolated by recombinant means, such as, but not limited to, antibodies isolated from an animal that is transgenic or transchromosomal or a hybridoma prepared therefrom, antibodies isolated from a host cell transformed to express the antibody (a transfectoma), antibodies isolated from a recombinant or combinatorial antibody library, and antibodies prepared or created by any means involving the splicing of immunoglobulin gene sequences to other DNA sequences. Such recombinant antibodies may be human recombinant antibodies. Such recombinant antibodies can be subjected to in vitro mutagenesis or in vivo somatic mutagenesis.

As used herein, the term “subject” refers to an animal. Preferably, the animal is a mammal. A subject also refers to for example, primates (e.g., humans), cows, sheep, goats, horses, dogs, cats, rabbits, rats, mice, fish, birds and the like. In a preferred embodiment, the subject is a human.

As used herein, the term “therapeutically effective amount” refers to an amount of an antibody of the disclosure that is sufficient to achieve a beneficial or desired result, including a clinical result. As such, the “therapeutically effective amount” may be sufficient, for example, to reduce or ameliorate the severity and/or duration of a SARS-CoV-2 infection, or one or more symptoms thereof, prevent the recurrence, development, or onset of one or more symptoms associated with a SARS-CoV-2 infection, prevent or reduce the replication or multiplication of SARS-CoV, prevent or reduce the production and/or release of a SARS-CoV-2 particle, or enhance or otherwise improve the prophylactic or therapeutic effect(s) of another therapy used in treating a SARS-CoV-2 infection. In certain embodiments, a “therapeutically effective amount” is an amount of the antibody of the disclosure that avoids or substantially attenuates undesirable side effects.

In certain embodiments, the “therapeutically effective amount” in the context of a SARS-CoV-2 infection is an amount sufficient to reduce one or more of the following steps of a the life cycle of SARS-CoV-2: the docking of the virus particle to a cell, the introduction of viral genetic information into a cell, the expression of viral proteins, the translation of viral RNA, the transcription of viral RNA, the replication of viral RNA, the synthesis of new viral RNA, the production of new virus particles and the release of virus particles from a cell. Such a reduction in any of the foregoing may be by at least 5%, preferably at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100%. In some embodiments, the “therapeutically effective amount” in the context of a SARS-CoV-2 infection reduces the replication, multiplication or spread of the virus by at least 5%, preferably at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100%. In some embodiments, the “therapeutically effective amount” in the context of a SARS-CoV-2 infection increases the survival rate of infected subjects by at least 5%, preferably at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100%. In each of the foregoing, when a reduction of increase is specified, such reduction of increase may be determined with respect to a subject that has not been treated with an antibody of the disclosure and that has a diagnosed SARS-CoV-2 infection.

As used herein, the terms “treating” or “treatment” mean an approach for obtaining beneficial or desired results, including clinical results. Beneficial or desired clinical results in the context of a SARS-CoV-2 infection include, but are not limited to, alleviation or amelioration of one or more symptoms or conditions, a diminution of extent of disease, a stabilized (i.e., not worsening) state of disease, preventing spread of disease, delay or slowing of disease progression, amelioration or palliation of the disease state and remission (whether partial or total), whether detectable or undetectable. “Treatment” or “treating” can also mean prolonging survival as compared to expected survival if not receiving treatment.

As used herein, the terms “a”, “an”, “the” and similar terms used in the context of the disclosure (especially in the context of the claims) are to be construed to cover both the singular and plural unless otherwise indicated herein or clearly contradicted by the context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided herein is intended merely to better illuminate the invention and does not pose a limitation on the scope of the invention otherwise claimed. No language in the specification should be construed as indicating any non-claimed element essential to the practice of the invention.

### Human Neutralizing Antibodies

The disclosure provides the following neutralizing SARS-CoV-2 antibodies that specifically binds to the spike protein of SARS-CoV-2: 1232D5, 1235C10, 1242C6, 1242D11, 1242E6, 1242F4, 1242F11, 1242G6, 1246C2, 1246H7, 1249A8, 1250D2, 1250E10, 1213H7, and 1212C2. In certain embodiments, an antigen binding fragment of the antibodies 1232D5, 1235C10, 1242C6, 1242D 11, 1242E6, 1242F4, 1242F11, 1242G6, 1246C2, 1246H7, 1249A8, 1250D2, 1250E10, 1213H7, and 1212C2 is provided. In certain embodiments, the antibodies are human monoclonal antibodies and/or the antigen binding fragments are derived from human monoclonal antibodies. Table 1 provides the SEQ ID NOS: for the nucleotide sequence (NT) of the V_{H} and V_{L} of 1232D5, 1235C10, 1242C6, 1242D11, 1242E6, 1242F4, 1242F11, 1242G6, 1246C2, 1246H7, 1249A8, 1250D2, 1250E10, 1213H7, and 1212C2 mAbs.

TABLE 1

SEQ ID NO:

| mAb | V_{H} | V_{L} |
|---|---|---|
| 1232D5 | 01 | 02 |
| 1235C10 | 03 | 04 |
| 1242C6 | 05 | 06 |
| 1242D11 | 07 | 08 |
| 1242E6 | 09 | 10 |
| 1242F4 | 11 | 12 |
| 1242F11 | 13 | 14 |
| 1242G6 | 15 | 16 |
| 1246C2 | 17 | 18 |
| 1246H7 | 19 | 20 |
| 1249A8 | 21 | 22 |
| 1250D2 | 23 | 24 |
| 1250E10 | 25 | 26 |
| 1213H7 | 27 | 28 |
| 1212C2 | 29 | 30 |

In a first embodiment, the disclosure provides an isolated antibody, or an antigen-binding fragment thereof, that specifically binds to the spike protein of SARS-CoV-2, the isolated antibody, or the antigen-binding fragment thereof, comprising: (i) a heavy chain variable region comprising the amino acid sequence selected from the group consisting of SEQ ID NOS: 01, 03, 05, 07, 09, 11, 13, 15, 17, 19, 21, 23, 25, 27 and 29, and (ii) a light chain variable region comprising the amino acid sequence selected from the group consisting of SEQ ID NOS: 02, 04, 06, 08, 10, 12, 14, 16, 18, 20, 22, 24, 26, 28 and 30; or a pharmaceutically acceptable form of any of the foregoing, such as, but not limited to a pharmaceutically acceptable salt, solvate and/or hydrate.

In a second embodiment, the disclosure provides an isolated antibody, or an antigen-binding fragment thereof, that specifically binds to the spike protein of SARS-CoV-2, the isolated antibody, or the antigen-binding fragment thereof, comprising: (i) a heavy chain variable region comprising the amino acid sequence selected from the group consisting of SEQ ID NOS: 01, 03, 05, 07, 09, 11, 13, 15, 17, 19, 21, 23, 25, 27 and 29, or an amino acid sequence at least 80% homologous thereto and (ii) a light chain variable region comprising the amino acid sequence selected from the group consisting of SEQ ID NOS: 02, 04, 06, 08, 10, 12, 14, 16, 18, 20, 22, 24, 26, 28 and 30, or an amino acid sequence at least 80% homologous thereto; or a pharmaceutically acceptable form of any of the foregoing, such as, but not limited to a pharmaceutically acceptable salt, solvate and/or hydrate.

In one aspect of any of the antibodies of the first and second embodiments, the antibody is provided as a pharmaceutically acceptable salt.

In one aspect of any of the antibodies of the first to second embodiments, the antibodies bind an epitope within the RBD of the SARS-CoV-2 spike protein. In one aspect of any of the antibodies of the first to second embodiments, the antibodies bind an epitope within the RBM of the SARS-CoV-2 spike protein. In one aspect of any of the antibodies of the first to second embodiments, the antibodies bind an epitope within the S2 region of the SARS-CoV-2 spike protein.

In one aspect of any of the antibodies of the first to second embodiments, the antibodies reduce binding of SARS-CoV-2 to a target cell. In one aspect of any of the antibodies of the first to second embodiments, the antibodies reduce cellular fusion between SARS-CoV-2 and a target cell. In one aspect of any of the antibodies of the first to second embodiments, the antibodies reduce release of infective SARS-CoV-2 from an infected cell. In one aspect of any of the antibodies of the first to second embodiments, the antibodies reduce infection of a target cell by SARS-CoV-2.

In one aspect of any of the antibodies of the first to second embodiments, the antibody is an antibody variant. In one aspect of any of the antibodies of the first to second embodiments, the antibody is an antibody variant comprising one or more substitutions, deletions, and/or insertions relative to the parental antibody. In one aspect of any of the antibodies of the first to second embodiments, the antibody is an antibody variant comprising one or more substitutions relative to the parental antibody.

In one aspect of any of the antibodies of the first to second embodiments, the antibody is an antibody variant that has a longer half-life in vivo in a subject relative to the parental antibody, decreased immunogenicity in vivo in a subject relative to the parental antibody, or a combination of the foregoing.

In one aspect of any of the antibodies of the first to second embodiments, the antibody comprises a variant Fc constant region. In one aspect of any of the antibodies of the first to second embodiments, the antibody comprises a variant Fc constant region, wherein a protein moiety or non-protein moiety is linked to the Fc constant region. In one aspect of any of the antibodies of the first to second embodiments, the antibody comprises a variant Fc constant region, wherein a water soluble polymer is linked to the Fc constant region. In one aspect of any of the antibodies of the first to second embodiments, the antibody comprises a variant Fc constant region, wherein a polyethylene glycol polymer is linked to the Fc constant region. In one aspect of any of the antibodies of the first to second embodiments, the antibody comprises a variant Fc constant region, wherein a polyoxazoline polymer is linked to the Fc constant region. In one aspect of any of the antibodies of the first to second embodiments, the antibody comprises a variant Fc constant region, wherein the variant Fc constant region provides a longer half-life in vivo in a subject relative to the parental antibody, decreased immunogenicity in vivo in a subject relative to the parental antibody, or a combination of the foregoing.

In one aspect of any of the antibodies of the first to second embodiments, the antibody is a human antibody. In one aspect of any of the antibodies of the first to second embodiments, the antibody is a chimeric antibody. In one aspect of any of the antibodies of the first to second embodiments, the antibody is a class-switched antibody.

In one aspect of any of the antibodies of the first to second embodiments, the antibody is linked to a therapeutic agent. In one aspect of any of the antibodies of the first to second embodiments, the antibody is linked to a detectable label. In one aspect of any of the antibodies of the first to second embodiments, the antibody is linked to an enzyme. In one aspect of any of the antibodies of the first to second embodiments, the antibody is linked to an enzyme inhibitor.

In one aspect of any of the antibodies of the first to second embodiments, the antibody is an antigen-binding fragment. In one aspect of any of the antibodies of the first to second embodiments, the antibody is an antigen-binding fragment selected from the groups consisting of: a Fab fragment, a F(ab)2 fragment, a Fab′ fragment, a Fd fragment, a Fv fragment, a disulfide-linked Fv (sdFv), a dAb fragment, an isolated CDR, a nanobody or single domain antibody, a portion of the V_{H} region containing a single variable domain and two constant domains, a diabody, a triabody, a tetrabody, scFv, scFv-FC, scFv-CH, scFab, and scFv-zipper.

### Antibody Fragments

In certain embodiments, an antibody of the disclosure is an antigen-binding fragment. Antigen-binding fragment include, but are not limited to, Fab, Fab′, Fab′-SH, F(ab)2, F(ab′)2, Fv, Fd, sdFv, dAb scFv fragments (including, but not limited to, scFv-FC, scFv-CH, scFab, and scFv-zipper), diabodies, triabodies tetrabodies, nanobodies, and other fragments described here. For discussion of Fab and F(ab)₂ fragments comprising salvage receptor binding epitope residues and having increased in vivo half-life, see U.S. Pat. No. 5,869,046.

Antibody fragments can be made by various techniques, including but not limited to proteolytic digestion of an intact antibody as well as production by recombinant host cells as described herein or as known in the art.

### Chimeric Antibodies

In certain embodiments, an antibody of the disclosure is a chimeric antibody. In one embodiment, a chimeric antibody comprises a non-human variable region (for example, a variable region derived from a mouse, rat, hamster, rabbit, or a monkey or other non-human primate) and a human constant region. In another embodiment, a chimeric antibody is a “class switched” antibody in which the class or subclass of the antibody has been changed from that of the parent antibody. Chimeric antibodies include antigen-binding fragments thereof. In certain embodiments, a chimeric antibody is a humanized chimeric antibody.

### Human Antibodies

In certain embodiments, an antibody of the disclosure is a human antibody. Human antibodies can be produced using various techniques known in the art or using techniques described herein. Human antibodies are described generally in van Dijk and van de Winkel, Curr. Opin. Pharmacol. 5: 368-74 (2001) and Lonberg, Curr. Opin. Immunol. 20:450-459 (2008). Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic animals, the endogenous immunoglobulin loci have generally been inactivated. For review of methods for obtaining human antibodies from transgenic animals see: Lonberg, Nat. Biotech. 23:1117-1125 (2005); U.S. Pat. Nos. 6,075,181 and 6,150,584; 5,770,429; 7,041,870; and U.S. Patent Application Publication No. US 2007/0061900. Human variable regions from intact antibodies generated by such animals may be further modified, such as, but not limited to, by combining with a different human constant region.

Human antibodies can also be made by hybridoma-based methods. Human myeloma and mouse-human heteromyeloma cell lines for the production of human monoclonal antibodies are known in the art (Kozbor J. Immunol., 133: 3001 (1984); Brodeur et al., Monoclonal Antibody Production Techniques and Applications, pp. 51-63 (Marcel Dekker, Inc., New York, 1987); and Boerner et al., J. Immunol., 147: 86 (1991)). Human antibodies generated via human B-cell hybridoma technology are also known in the art (Li et al., Proc. Natl. Acad. Sci. USA, 103:3557-3562 (2006)). Additional methods include those described in U.S. Pat. No. 7,189,826; Ni, Xiandai Mianyixue, 26(4):265-268 (2006); Vollmers and Brandlein, Histology and Histopathology, 20(3):927-937 (2005); and Vollmers and Brandlein, Methods and Findings in Experimental and Clinical Pharmacology, 27(3):185-91 (2005). Human antibodies may also be generated by isolating Fv clone variable domain sequences selected from human-derived phage display libraries. Such variable domain sequences may then be combined with a desired human constant domain.

### Antibody Variants

The disclosure also provides for variants of the antibodies disclosed. In certain embodiments, amino acid sequence variants of the antibodies provided herein are contemplated. Such variants may be used to improve the binding affinity of an antibody, to improve a biological property of an antibody (such as, but not limited to, half-life), or a combination of the foregoing. Amino acid variants of an antibody may be prepared by introducing appropriate modifications into the nucleotide sequence encoding the antibody, or by peptide synthesis directly incorporating such amino acid change. Antibody variants include, but are not limited to, fusion proteins including an antibody of the disclosure, substitutions of one or more amino acids, deletion of one or more amino acids, insertion of one or more amino acids, and any combination of the foregoing. In certain embodiments, the antibody variant retains the ability to bind to the same epitope. In certain embodiments, antibody variants having one or more amino acid substitutions are provided. When a substitution is introduced, the substitution may be made at any desired location.

In certain embodiments, when a substitution is introduced, the substitution occurs in the Sites of interest for substitutional mutagenesis include the V_{H} region, the V_{L} region, the heavy chain CDRs, the light chain CDRs, and/or the FR region. Such substitutions may be conservative or non-conservative. Conservative substitutions are defined herein.

In certain embodiments, an antibody of the disclosure comprises a conservative amino acid substitution. Suitably, such conservative amino acid substitution may be made in the V_{H} region, the V_{L} region, the heavy chain CDRs, the light chain CDRs, and/or the FR region. In certain embodiments, the disclosure provides for an antibody variant of one or more of the polypeptides of SEQ ID NOS: 01-30. In certain embodiments, the disclosure provides for an antibody variant comprising one or more substitutions, including conservative substitutions, of one or more of the polypeptides of SEQ ID NOS: 01-30.

In one embodiment of any of the foregoing, an antibody variant has an amino acid sequence that is at least 60%, 70%, 75%, 80%, 85%, 90%, 95%, 96%, 97%, 98%, or 99% identical to a parent antibody.

For the purposes of the disclosure, the percent homology between two amino acid sequences is equivalent to the percent identity between the two sequences. The percent identity between the two sequences is a function of the number of identical positions shared by the sequences (% homology=(number of identical positions/total number of positions)×100), taking into account the number of gaps, and the length of each gap, which need to be introduced for optimal alignment of the two sequences. The percent identity between two amino acid sequences is preferably determined using the algorithm of E. Meyers and W. Miller (Comput. Appl. Biosci., 4:11-17 (1988)) which has been incorporated into the ALIGN program (version 2.0), using a PAM120 weight residue table, a gap length penalty of 12 and a gap penalty of 4.

In one embodiment, an antibody variant includes a conservative modification. As used herein, the term “conservative modifications” refers to an amino acid modification that does not significantly affect or alter the binding characteristics of the antibody containing the conservative modification. Such conservative modifications include amino acid substitutions, additions, and deletions.

In one embodiment, an antibody variant contains a conservative amino acid substitution. Conservative amino acid substitutions are ones in which an amino acid residue is replaced with an amino acid residue having a similar side chain. Families of amino acid residues having similar side chains have been defined in the art. These families include: amino acids with basic side chains (lysine, arginine, and histidine), acidic side chains (aspartic acid and glutamic acid), uncharged polar side chains (glycine, asparagine, glutamine, serine, threonine, tyrosine, cysteine, and tryptophan), nonpolar side chains (alanine, valine, leucine, isoleucine, proline, phenylalanine, and methionine), beta-branched side chains (threonine, valine, and isoleucine), and aromatic side chains (tyrosine, phenylalanine, tryptophan, and histidine). Non-conservative substitutions will entail exchanging a member of one of these classes for another class.

For example, a conservative amino acid substitution may involve a substitution of a native amino acid residue with a nonnative residue such that there is little or no effect on the polarity, steric bulk, charge, hydrophobicity and/or hydrophilicity of the amino acid residue at that position. Conservative amino acid substitutions also encompass non-naturally occurring amino acid residues which are typically incorporated by chemical peptide synthesis rather than by synthesis in biological systems. These include peptidomimetics, and other reversed or inverted forms of amino acid moieties. It will be appreciated by those of skill in the art that polypeptide described herein may be chemically synthesized as well as produced by recombinant means.

In making an amino acid substitution as described herein, the hydropathic index of an amino acid may be considered. Each amino acid has been assigned a hydropathic index on the basis of their hydrophobicity and charge characteristics. Hydropathic index values are resented by: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

The importance of the hydropathic amino acid index in conferring interactive biological function on a protein is understood in the art (Kyte et al., J. Mol. Biol., 157:105-131, 1982). It is known that certain amino acids may be substituted for other amino acids having a similar hydropathic index or score and still retain a similar biological activity. In one embodiment, making changes based upon the hydropathic index, the substitution of amino acids whose hydropathic indices are within +/−1; in an alternate embodiment, the hydropathic indices are within +/−0.5; in yet another alternate embodiment, the hydropathic indices are within +/−0.25.

In making an amino acid substitution as described herein, the hydrophilicity may also be considered. In certain embodiments, the greatest local average hydrophilicity of a polypeptide as governed by the hydrophilicity of its adjacent amino acids, correlates with a biological property of the protein. The following hydrophilic index values have been assigned to amino acid residues: arginine (+3.0); lysine (+3.0); aspartate (+3.0.+−0.1); glutamate (+3.0.+−0.1); serine (+0.3); asparagine (+0.2); glutamine (+0.2); glycine (0); threonine (−0.4); proline (−0.5.+−0.1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4).

In one embodiment, in making changes based upon similar hydrophilicity values, the substitution of amino acids whose hydrophilicity values are within +/−1; in an alternate embodiment, the hydrophilicity values are within +/−0.5; in yet another alternate embodiment, the hydrophilicity values are within +/−0.25.

A skilled artisan will be able to determine suitable substitutions, insertions and deletions, including combinations thereof, of a polypeptide as set forth in any of SEQ ID NOS: 01-30 using techniques known in the art. For identifying suitable areas of a polypeptide that may be changed without destroying activity, one skilled in the art may target areas not believed to be important for activity. For example, when homologous polypeptides with similar activities from the same species or from other species are known, one skilled in the art may compare the amino acid sequence of a polypeptide described herein to such homologous polypeptides. With such a comparison, one can identify residues and portions of the molecules that are conserved among similar polypeptides. It will be appreciated that changes in areas of a polypeptide described herein that are not conserved relative to such homologous polypeptide would be less likely to adversely affect the biological activity and/or structure of a polypeptide described herein. One skilled in the art would also know that, even in relatively conserved regions, one may substitute chemically similar amino acids for the naturally occurring residues while retaining activity (for example, conservative amino acid substitutions). Therefore, even areas that may be important for biological activity or for structure may be subject to such amino acid substitutions without destroying the biological activity or without adversely affecting the polypeptide structure.

The deletions, insertions, and substitutions can be selected, as would be known to one of ordinary skill in the art, to generate a desired polypeptide variants. For example, it is not expected that deletions, insertions, and substitutions in a non-functional region of a polypeptide would alter activity. Likewise conservative amino acid substitutions and/or substitution of amino acids with similar hydrophilic and/or hydropathic index values is expected to be tolerated in a conserved region and a polypeptide activity may be conserved with such substitutions.

An exemplary substitution variant is an affinity matured antibody, which may be conveniently generated using phage display-based affinity maturation techniques such as those described in Hoogenboom et al., in Methods in Molecular Biology 178:1-37 (O'Brien et al., ed., Human Press, Totowa, N.J., (2001)). Amino acid sequence insertions include amino- and/or carboxyl-terminal fusions ranging in length from one residue to polypeptides containing a hundred or more residues, as well as intra-sequence insertions of single or multiple amino acid residues. Examples of terminal insertions include an antibody with an N-terminal methionyl residue. Other insertional variants of the antibody molecule include the fusion to the N- or C-terminus of the antibody to an enzyme or a polypeptide which increases the serum half-life of the antibody.

An antibody variant may be screened for a desired activity, including, but not limited to, retained epitope binding, improved epitope binding, decreased immunogenicity, improved antibody-dependent cellular cytotoxicity (ADCC), complement-dependent cytotoxicity (CDC) or any combination of the foregoing.

An antibody of the disclosure may be modified to contain additional protein moieties and additional moieties that are not protein based. In one embodiment, a non-protein based moiety is a polymer, preferable a water soluble polymer. Suitable water soluble polymers include, but are not limited to, polyethylene glycol (PEG), polyoxazoline (POZ), carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either as a homopolymer or a copolymer), and dextran or poly(n-vinyl pyrrolidone)polyethylene glycol, propylene glycol, polyoxyethylated polyols (such as glycerol), polyvinyl alcohol, copolymers of ethylene glycol and propylene glycol, copolymers of propylene oxide and ethylene oxide, and mixtures of any of the foregoing. When the water soluble polymer is a copolymer, the copolymer may be present as a block copolymer or a random copolymer. An amino acid substitution may be used to introduce a site for attachment of a water soluble polymer to the antibody.

The water soluble polymer may be of any molecular weight, and may be dendrimers, branched or unbranched. The number of polymers attached to an antibody may vary. In one embodiment, the number average molecular weight of the water soluble polymer is from 2,500 to 75,000 Da, from 5,000 to 50,000 Da, from 7,500 to 40,000 Da, or from 10,000 to 30,000 Da. In one embodiment, 1 to 10 water soluble polymer chains are attached. When more than 1 water soluble polymer is attached, the water soluble polymers may be the same or different and may be of the same of different number average molecular weight. In one embodiment, the water soluble polymers increases the half-life of an antibody of the disclosure and/or decrease immunogenicity of an antibody of the disclosure. Water soluble polymers may be linked to an antibody of the disclosure using conventional reactive groups on the polymer and the antibody. For example, water soluble polymers, such as. But not limited to, POZ and PEG polymers, may be linked to an antibody using acylation alkylation reactions. Water soluble polymers may be linked to the antibody in a site specific manner or randomly (for example, the g-amino group of a lysine residue or the thiol group of cysteine residue may be used in conjugation reactions with an appropriate functionality on the water soluble polymer). In certain embodiments, a non-natural amino acid may be introduced into the antibody and used to link a water soluble polymer to the antibody. For example, a selenocysteine residue may be introduced into the antibody for reaction with a water soluble polymer containing an appropriate functionality (for example, a maleimide group or an iodoacetimide group). In certain embodiments, the water soluble polymer may be linked to a therapeutic agent, a detectable label, an enzyme, or an enzyme inhibitor as described herein.

Methods for linking water soluble polymers to proteins are known in the art and may be applied directly to the antibodies of the disclosure (EP 0154316).

In another embodiment, a non-protein based moiety is a therapeutic agent, for example a therapeutic agent useful in treating a SARS-CoV-2 infection as described herein or a cytotoxic agent. In another embodiment, a non-protein based moiety is a detectable label. In another embodiment, a non-protein based moiety is an enzyme. In another embodiment, a non-protein based moiety is an enzyme inhibitor, for example a serine protease inhibitor, such as, but not limited to, a TMPRSS2 inhibitor.

### Glycosylation Variants

In certain embodiments, an antibody of the disclosure is altered to increase or decrease the extent to which the antibody is glycosylated. Addition or deletion of glycosylation sites to an antibody may be conveniently accomplished by altering the amino acid sequence such that one or more glycosylation sites are created or removed. For example, an aglycoslated antibody can be made. Glycosylation can be altered to for a variety of purposes, including, but not limited to, to increase the affinity of the antibody for an antigen. In one embodiment, one or more amino acid substitutions are made that result in elimination of one or more FR glycosylation sites, which may increase the affinity of the antibody for antigen. Such an approach is described in further detail in U.S. Pat. Nos. 5,714,350 and 6,350,861. Glycosylation sites in the constant region may also be eliminated. For example, in IgG1, N297 in the Fc portion may be substituted with another residue (for example, alanine) and/or by mutating an adjacent amino acid to thereby reduce glycosylation on N297.

Additionally or alternatively, an antibody can be made that has an altered type of glycosylation, such as a hypofucosylated antibody having reduced amounts of fucosyl residues or an antibody having increased bisecting GlcNac structures. Such altered glycosylation patterns have been demonstrated to increase the ADCC ability of antibodies. Such carbohydrate modifications can be accomplished, for example, by expressing the antibody in a host cell with altered glycosylation machinery (such cells are described in EP 1,176,195; PCT Publication WO 03/035835; Shields, R. L. et al. (2002), J. Biol. Chem. 277:26733-26740; PCT Publication WO 99/54342; Umana et al. (1999), Nat. Biotech. 17: 176-180).

### Fc Region Variants

The numbering of residues in the Fc region as discussed herein is that of the EU index of Kabat (PCT Publication WO 2000/42072). The variable regions of the antibody described herein can be linked to an Fc region (such as, but not limited to, an IgG1, IgG2, IgG3 or IgG4 Fc), which may be of any allotype or isoallotype (including for IgG1: G1m, G1m1(a), G1m2(x), G1m3(f), G1m17(z); for IgG2: G2m, G2m23(n); for IgG3: G3m, G3m21(g1), G3m28(g5), G3 ml 1(b0), G3m5(b1), G3m13(b3), G3m14(b4), G3m10(b5), G3m15(s), G3m16(t), G3m6(c3), G3m24(c5), G3m26(u), G3m27(v); for IgA: A2M, A2 ml, and A2m2; and for K: Km, Km1, Km2, Km3 (Jefferies et al. (2009) mAbs 1(4), 323-338). In certain embodiments, the variable regions of the antibodies described herein are linked to an Fe that binds to one or more activating Fc receptors (FcR), and thereby stimulate ADCC. In certain embodiments, the variable regions of the antibodies described herein are linked to an Fc region optimized to engage a wider range of Fc receptors. Fc receptors for isotypes other than gamma exist on particular leukocytes. By creating an Fc region that can interact with multiple Fc receptors, such as FcγRI and FcαRI, an antibody with expanded, novel abilities to engage effector cells may be created. Neutrophils are the most abundant leukocyte in the body and engage Fc of IgA antibodies via the FcαRI.

In certain embodiments, the antibody variable regions described herein may be linked to an Fc comprising one or more modification, typically to alter one or more functional properties of the antibody, such as serum half-life, complement fixation, Fc receptor binding, ADCC, and/or CDC.

The Fc region encompasses domains derived from the constant region of an antibody. Suitable immunoglobulins include IgG 1, IgG2, IgG3, IgG4, IgA1, IgA2, and other classes such as IgD, IgE and IgM, The constant region of an antibody is defined as a naturally-occurring or synthetically-produced polypeptide homologous to the antibody C-terminal region, and can include a CH1 domain, a hinge, a CH2 domain, a CH3 domain, or a CH4 domain, separately or in combination. In some embodiments, an antibody of the disclosure has an Fc region other than that of a wild type IgA1. An antibody of the disclosure may have an Fc region from that of IgG (e.g., IgG1, IgG2, IgG3, and IgG4) or other classes such as IgA2, IgD, IgE and IgM. An antibody of the disclosure may have an Fc region that is contains a substitution, deletion, or insertion of wild-type IgA1.

The Fc of an antibody is responsible for many important functions including FcR binding and complement fixation. There are five major classes of heavy chain constant regions, classified as IgA, IgG, IgD, IgE, and IgM, each with characteristic effector functions designated by isotype. The serum half-life of an antibody is influenced by the ability of that antibody to bind to an FcR.

Antibody molecules interact with multiple classes of cellular receptors. IgG molecules interact with three classes of FcγR specific for the IgG class of antibody, namely FcγRI, FcγRIIa, FcγRIIb, FcγRIIIa, and FcγRIIIb. The important sequences for the binding of IgG to the FcγR receptors have been reported to be located in the CH2 and CH3 domains.

In certain embodiments, the Fc region is a variant Fc region (an Fc sequence that has been modified such as by amino acid substitution, deletion and/or insertion) relative to a parent Fc sequence to provide desirable structural features and/or biological activity, including, but not limited to, (i) increased or decreased ADCC; (ii) increased or decreased CDC; (iii) increased or decreased affinity for Clq; and/or (iv) increased or decreased affinity for a FcR (each of the foregoing relative to the parent Fc). Such Fc region variants will generally comprise at least one amino acid substitution, deletion, and/or insertion in the Fc region. In certain embodiments, an Fc region contains from 1 to 6 amino acid substitutions, deletions and/or insertions (preferably substitutions).

A variant Fc region may also comprise a sequence modification wherein an amino acid involved in disulfide bond formation are removed or replaced with another amino acid. In other embodiments, the Fc region may be modified to make it more compatible with a selected host cell. For example, one may remove the PA sequence near the N-terminus of a typical native Fc region, which may be recognized by a digestive enzyme in E. coli such as proline iminopeptidase. In other embodiments, one or more glycosylation sites within the Fe domain are removed. Residues that are typically glycosylated (e.g., asparagine) may confer cytolytic response. Such residues may be deleted or substituted with unglycosylated residues (e.g., alanine). In other embodiments, sites involved in interaction with complement, such as the Clq binding site, may be removed from the Fc region. For example, one may delete or substitute the EKK sequence of human IgG1. In certain embodiments, sites that affect binding to Fc receptors may be removed, preferably sites other than salvage receptor binding sites. In other embodiments, an Fc region may be modified to remove an ADCC site known in the art. Specific examples of variant Fe domains are disclosed for example, in WO 1997/34631 and WO 1996/32478.

In one embodiment, the hinge region of Fe is modified such that the number of cysteine residues in the hinge region is increased or decreased (U.S. Pat. No. 5,677,425) to, for example, facilitate assembly of the light and heavy chains and/or to increase or decrease the stability of the antibody. In one embodiment, the Fe hinge region of an antibody is mutated to decrease the biological half-life of the antibody. More specifically, one or more amino acid mutations are introduced into the C_{H2}-CH3 domain interface region of the Fc-hinge fragment such that the antibody has impaired Staphylococcal protein A (SpA) binding relative to native Fc-hinge domain SpA binding (U.S. Pat. No. 6,165,745).

In yet other embodiments, the Fc region is altered by replacing at least one amino acid residue with a different amino acid residue to alter an effector function of the antibody. For example, in an IgG antibody one or more amino acids selected from amino acid residues 234, 235, 236, 237, 297, 318, 320 and 322 can be replaced with a different amino acid residue such that the antibody has an altered affinity for an effector ligand but retains the antigen-binding ability of the parent antibody. The effector ligand to which affinity is altered can be, for example, an FcR or the CI component of complement (U.S. Pat. Nos. 5,624,821 and 5,648,260). In another example, in an IgG antibody one or more amino acids selected from amino acid residues 329, 331 and 322 can be replaced with a different amino acid residue such that the antibody has altered Clq binding and/or reduced or abolished CDC (U.S. Pat. No. 6,194). In another example, in an IgG antibody one or more amino acid residues within amino acid positions 231 and 239 are altered to thereby alter the ability of the antibody to fix complement (PCT Publication WO 1994/29351). All references to Fc region amino acid numbering is made according to the EU index of Kabat (Kabat et al., (1983) “Sequences of Proteins of Immunological Interest”, US Dept. Health and Human Services).

In yet another example, the Fc region may be modified to increase ADCC and/or to increase the affinity for an FcγR by modifying one or more amino acids at the following positions: 234, 235, 236, 238, 239, 240, 241, 243, 244, 245, 247, 248, 249, 252, 254, 255, 256, 258, 262, 263, 264, 265, 267, 268, 269, 270, 272, 276, 278, 280, 283, 285, 286, 289, 290, 292, 293, 294, 295, 296, 298, 299, 301, 303, 305, 307, 309, 312, 313, 315, 320, 322, 324, 325, 326, 327, 329, 330, 331, 332, 333, 334, 335, 337, 338, 340, 360, 373, 376, 378, 382, 388, 389, 398, 414, 416, 419, 430, 433, 434, 435, 436, 437, 438 or 439. Exemplary substitutions include 236A, 239D, 239E, 268D, 267E, 268E, 268F, 324T, 332D, and 332E. Exemplary variants include 239D/332E, 236A/332E, 236A/239D/332E, 268F/324T, 267E/268F, 267E/324T, and 267E/268F7324T. Other modifications for enhancing FcγR and complement interactions include but are not limited to substitutions 298A, 333A, 334A, 326A, 2471, 339D, 339Q, 280H, 290S, 298D, 298V, 243L, 292P, 300L, 396L, 3051, and 396L. These and other modifications are described in Strohl, 2009, Current Opinion in Biotechnology 20:685-691.

Fc modifications that increase binding to an FcγR include amino acid modifications at any one or more of amino acid positions 238, 239, 248, 249, 252, 254, 255, 256, 258, 265, 267, 268, 269, 270, 272, 279, 280, 283, 285, 298, 289, 290, 292, 293, 294, 295, 296, 298, 301, 303, 305, 307, 312, 315, 324, 327, 329, 330, 335, 337, 3338, 340, 360, 373, 376, 379, 382, 388, 389, 398, 414, 416, 419, 430, 434, 435, 437, 438 or 439 of the Fc region.

Other Fc modifications that can be made are those for reducing or ablating binding to FcγR and/or complement proteins, thereby reducing or ablating Fc-mediated effector functions such as ADCC, ADCP, and CDC. Exemplary modifications include but are not limited substitutions, insertions, and deletions at positions 234, 235, 236, 237, 267, 269, 325, and 328, wherein numbering is according to the EU index of Kabat. Exemplary substitutions include but are not limited to 234G, 235G, 236R, 237K, 267R, 269R, 325L, and 328R, wherein numbering is according to the EU index of Kabat. An Fc variant may comprise 236R/328R. Other modifications for reducing FcγR and complement interactions include substitutions 297A, 234A, 235A, 237A, 318A, 228P, 236E, 268Q, 309L, 330S, 331S, 220S, 226S, 229S, 238S, 233P, and 234V, as well as removal of the glycosylation at position 297 by mutational or enzymatic means or by production in organisms such as bacteria that do not glycosylate proteins. These and other modifications are reviewed in Strohl, 2009, Current Opinion in Biotechnology 20:685-691.

Optionally, the Fc region may comprise a non-naturally occurring amino acid residue at additional and/or alternative positions known to one skilled in the art (U.S. Pat. Nos. 5,624,821; 6,277,375; 6,737,056; 6,194,551; 7,317,091; 8,101,720; PCT Publication Nos: WO 2000/42072; WO 2001/58957; WO 2002/06919; WO 2004/016750; WO 2004/029207; WO 2004/035752; WO 2004/074455; WO 2004/099249; WO 2004/063351; WO 2005/070963; WO 2005/040217, WO 2005/092925 and WO 2006/020114).

Fc variants that enhance affinity for an inhibitory receptor FcγRIIb may also be used. Such variants may provide an Fc fusion protein with immune-modulatory activities related to FcγRIIb cells, including for example B cells and monocytes. In one embodiment, the Fc variants provide selectively enhanced affinity to FcγRIIb relative to one or more activating receptors. Modifications for altering binding to FcγRIIb include one or more modifications at a position selected from the group consisting of 234, 235, 236, 237, 239, 266, 267, 268, 325, 326, 327, 328, and 332, according to the EU index of Kabat. Exemplary substitutions for enhancing FcγRIIb affinity include but are not limited to 234D, 234E, 234F, 234W, 235D, 235F, 235R, 235Y, 236D, 236N, 237D, 237N, 239D, 239E, 266M, 267D, 267E, 268D, 268E, 327D, 327E, 328F, 328W, 328Y, and 332E. Exemplary substitutions include 235Y, 236D, 239D, 266M, 267E, 268D, 268E, 328F, 328W, and 328Y. Other Fc variants for enhancing binding to FcγRIIb include 235Y/267E, 236D/267E, 239D/268D, 239D/267E, 267E/268D, 267E/268E, and 267E/328F.

The affinities and binding properties of an Fc region for its ligand may be determined by a variety of in vitro assay methods (biochemical or immunological based assays) known in the art including but not limited to, equilibrium methods (enzyme-linked immune-absorbent assay, radioimmunoassay, or kinetics, and other methods such as indirect binding assays, competitive inhibition assays, fluorescence resonance energy transfer, gel electrophoresis and chromatography). These and other methods may utilize a label on one or more of the components being examined and/or employ a variety of detection methods including but not limited to chromogenic, fluorescent, luminescent, or isotopic labels. A detailed description of binding affinities and kinetics can be found in Paul, W. E., ed., Fundamental immunology, 4th Ed., Lippincott-Raven, Philadelphia (1999), which focuses on antibody-immunogen interactions.

In certain embodiments, the antibody is modified to increase its biological half-life. Various approaches are possible. For example, this may be done by increasing the binding affinity of the Fc region for FcRn. For example, one or more of following residues can be mutated: 252, 254, 256, 433, 435, and 436, as described in U.S. Pat. No. 6,277,375. Specific exemplary substitutions include one or more of the following: T252L, T254S, and/or T256F. Alternatively, to increase the biological half-life, the antibody can be altered within the CH1 or CL region to contain a salvage receptor binding epitope taken from two loops of a CH2 domain of an Fc region of an IgG, as described in U.S. Pat. Nos. 5,869,046 and 6,121,022. Other exemplary variants that increase binding to FcRn and/or improve pharmacokinetic properties include substitutions at positions 259, 308, 428, and 434, including for example 2591, 308F, 428L, 428M, 434S, 434H. 434F, 434Y, and 434M. Other variants that increase Fc binding to FcRn include: 250E, 250Q, 428L, 428F, 250Q/428L (Hinton et al. 2004, J. Biol. Chem. 279(8): 6213-6216, Hinton et al. 2006 Journal of Immunology 176:346-356), 256A, 272A, 286A, 305A, 307A, 307Q, 311A, 312A, 376A, 378Q, 380A, 382A, 434A (Shields et al, Journal of Biological Chemistry, 2001, 276(9):6591-6604), 252F, 252T, 252Y, 252W, 254T, 256S, 256R, 256Q, 256E, 256D, 256T, 309P, 311S, 433R, 433S, 4331, 433P, 433Q, 434H, 434F, 434Y, 252Y/254T/256E, 433K/434F/436H, 308T/309P/311S (Dall′ Acqua et al. Journal of Immunology, 2002, 169:5171-5180, Dall′ Acqua et al., 2006, Journal of Biological Chemistry 281:23514-23524). Other modifications for modulating FcRn binding are described in Yeung et al., 2010, J Immunol, 182:7663-7671.

In certain embodiments, hybrid IgG isotypes with particular biological characteristics may be used. For example, an IgG1/IgG3 hybrid variant may be constructed by substituting IgG1 positions in the CH2 and/or CH3 region with the amino acids from IgG3 at positions where the two isotypes differ. Thus a hybrid variant IgG antibody may be constructed that comprises one or more substitutions, e.g., 274Q, 276K, 300F, 339T, 356E, 358M, 384S, 392N, 397M, 4221, 435R, and 436F. In other embodiments described herein, an IgG1/IgG2 hybrid variant may be constructed by substituting IgG2 positions in the CH2 and/or CH3 region with amino acids from IgG1 at positions where the two isotypes differ. Thus a hybrid variant IgG antibody may be constructed chat comprises one or more substitutions, e.g., one or more of the following amino acid substitutions: 233E, 234L, 235L, 236G (referring to an insertion of a glycine at position 236), and 321 h.

Moreover, the binding sites on human IgG1 for FcγRI, FcγRII, FcγRIII and FcRn have been mapped and variants with improved binding have been described (see Shields, R. L. et al. (2001) J. Biol. Chem. 276:6591-6604). Specific mutations at positions 256, 290, 298, 333, 334 and 339 were shown to improve binding to FcγRIII Additionally, the following combination mutants were shown to improve FcγRIII binding: T256A/S298A, S298A/E333A, S298A/K224A and S298A/E333A/K334A, which has been shown to exhibit enhanced FcγRIIIa binding and ADCC activity (Shields et al., 2001). Other IgG1 variants with strongly enhanced binding to FcγRIIIa have been identified, including variants with S239D/I332E and S239D/I332E/A330L mutations which showed the greatest increase in affinity for FcγRIIIa, a decrease in FcγRIIb binding, and strong cytotoxic activity in cynomolgus monkeys (Lazar et al., 2006). Introduction of the triple mutations into antibodies such as alemtuzumab (CD52-specific), trastuzumab (HER2/neu-specific), rituximab (CD20-specific), and cetuximab (EGFR-specific) translated into greatly enhanced ADCC activity in vitro, and the S239D/I332E variant showed an enhanced capacity to deplete B cells in monkeys (Lazar et al., 2006). In addition, IgG1 mutants containing L235V, F243L, R292P, Y300L and P396L mutations which exhibited enhanced binding to FcγRIIIa and concomitantly enhanced ADCC activity in transgenic mice expressing human FcγRIIIa in models of B cell malignancies and breast cancer have been identified (Stavenhagen et al., 2007; Nordstrom et al., 2011). Other Fc mutants that may be used include, but are not limited to: S298A/E333A/L334A, S239D/1332E, S239D/1332E/A330L, L235V/F243L/R292P/Y300L/P396L, and M428L/N434S.

In certain embodiments, an Fe is chosen that has reduced binding to FcγRs. An exemplary Fc, e.g., IgG1 Fc, with reduced FcγR binding comprises the following three amino acid substitutions: L234A, L235E and G237A.

In certain embodiments, an Fc is chosen that has reduced complement fixation. An exemplary Fc, e.g., IgG1 Fc, with reduced complement fixation has the following two amino acid substitutions: A330S and P331S.

In certain embodiments, an Fc is chosen that has essentially no effector function, i.e., it has reduced binding to FcγRs and reduced complement fixation. An exemplary Fc, IgG1 Fc that is effectorless comprises the following five mutations: L234A, L235E, G237A, A330S and P331S. When using an IgG4 constant domain, it is usually preferable to include the substitution S228P, which mimics the hinge sequence in IgG1 and thereby stabilizes IgG4 molecules.

### Nucleic Acids, Vectors, and Host Cells

The disclosure also provides for nucleic acid sequences encoding the antibodies of the disclosure. The nucleic acid sequences may code for an antigen-binding fragment. In some embodiments, the nucleic acids are codon optimized based on how the antibodies are produced. In one embodiment, the nucleic acid sequence comprises one or more of SEQ ID NOS: 01 to 30. In one aspect of this embodiment, the nucleic acid sequence has at least 75% homology, at least 80% homology, at least 85% homology, at least 90% homology, at least 95% homology, or greater than 95% homology with one or more of SEQ ID NOS: 01 to 30.

The disclosure also provides a vector comprising a nucleic acid sequence coding for an antibody of the disclosure. In certain embodiments, a vector comprises a nucleic acid sequence coding for a variable heavy chain region and a nucleic acid sequence coding for a variable light chain region. Any nucleic acid sequence of the disclosure (SEQ ID NOS: 01-30) may be combined with a vector as described herein. In some embodiments, a nucleic acid sequence coding for a variable heavy chain is on the same vector as a nucleic acid sequence coding for a variable light chain. In some embodiments, a nucleic acid sequence coding for a variable heavy chain region is on a different vector than a nucleic acid sequence coding for a variable light chain region. In some embodiments, the vector is a plasmid. In some embodiments, the vector is a phage vector, such as, but not limited to, X-phage. In some embodiments, the vector is a viral vector, such as, but not limited to, non-replicating adenoviral vector, lentiviral vector, pSV, pCMV, and retroviral vectors. In some embodiments, the vector is a cosmid. In some embodiments, the vector is a recombinant chromosome. In some embodiments, the combinations of the foregoing vectors are employed. The expression of different nucleic acid sequences may occur at the same time generally or be temporally separated. The expression of one or more nucleic acid sequences may be inducible. The vector may comprise a nucleic acid coding for an intact antibody or an antigen binding fragment, particularly those antigen-binding fragments disclosed herein. In some embodiments, the vector comprises a nucleic acid sequence encoding an immunoglobulin constant region, such as, but not limited to, an IgG (e.g. IgG1, IgG2, IgG3, and IgG4) constant region. In exemplary embodiments, the immunoglobulin constant region comprises an IgG1 constant region.

In one embodiment, the disclosure provides for a vector comprising nucleotide sequences that code for the heavy and/or light chain variable regions of an antibody selected from the groups consisting of: 1232D5, 1235C10, 1242C6, 1242D11, 1242E6, 1242F4, 1242F11, 1242G6, 1246C2, 1246H7, 1249A8, 1250D2, 1250E10, 1213H7 and 1212C2. The vector may be a mammalian expression vectors, such that the vector may be transfected into mammalian cells and the DNA may be integrated into the genome by homologous recombination in the case of stable transfection, or alternatively the cells may be transiently transfected. Common to most engineered vectors are origin of replications, multicloning sites, and selectable markers. Common promoters for mammalian expression vectors include CMV and SV40 promoters, and non-viral promoters such as, but not limited to, EF-1 promoters. In certain embodiments, the disclosure provides a vector comprising one or more nucleic acid sequences encoding one or more CDRs of one or more heavy and/or light chains of one or more of the antibodies of the disclosure. In certain embodiments, the disclosure provides a vector comprising one or more nucleic acid sequences encoding one or more heavy and/or light chain variable regions of one or more of the antibodies of the disclosure.

The nucleic acid sequences may encode for an antibody variant as described herein, including an antibody containing a conservative substitution. A vector that codes for one or both variable region(s) of an antibody of the disclosure may contain one of or both of SEQ ID NOS: required to generate such antibody, or a nucleotide sequence that shares a degree minimum of homology with such SEQ ID NOS: (for example, at least 75%, at least 80%, at least 85%, at least 90%, at least 95%, or more than 95% homology).

The disclosure also provides for a cell transformed with a vector described herein. One of ordinary skill in the art will appreciate that different cell types may lead to different antibody products and may possibly impact the therapeutic efficacy of the antibody products, e.g. through having distinct variations in glycosylation patterns, especially N-linked glycosylation patterns. Such discussions may be found in Liu L, J Pharm Sci. 2015 June; 104(6):1866-84; Rosenlocher et al., J Proteomics. 2016 Feb. 16; 134:85-92; Mimura et al., J Immunol Methods. 2016 January; 428:30-6; and Croset et al., Journal of Biotechnology, 161(3), Oct. 31, 2012.

In some embodiments, the cell is a bacterial cell, a yeast cell, a plant cell, or a mammalian cell. In some embodiments, the mammalian cell is one of a Chinese hamster ovary (CHO) cell, including DUXB11, DG44 and CHOK1 lineages, a NS0 murine myeloma cell, a PER.C6 cell, and a human embryonic kidney (HEK) cell, including HEK293 lineages. Other less common host cells, include plant cells, for example, those based on the tumor-inducing (Ti) plasmid of Agrobacterium tumefaciens. Cell-free expression systems also exist, for example, based on E. coli cell lysate, containing cellular components necessary for transcription/translation. Eukaryotic and mammalian cell-free systems are also known in the art, for example wheat germ cell-free expression system. Some recombinant antibody production systems express the recombinant antibodies on the surface of the host cell before harvesting, others simply release the antibodies into a medium for collection. Such variations are intended to be within the scope of the disclosure.

The disclosure also provides for a method of making a recombinant antibody of the disclosure. In some embodiments, the antibody is an antigen binding fragment. The host cell comprising a vector described herein is induced to produce the recombinant antibodies and the host cell assembles the antibodies from heavy/light chains in the host cell and then transport the antibodies out of the cell, or the antibodies may self-assemble outside the host cell and be exported as heavy/light chains. An overview of cell culture processes for recombinant monoclonal antibody production may be found in Li et al., Mabs. 2010 September-October; 2(5): 466-477. In one embodiment, the disclosure provides for a method of making a recombinant antibody, or antigen-binding fragment thereof, that specifically binds to SARS-CoV-2 spike protein, the method comprising providing a cell comprising a vector comprising a nucleic acid sequence encoding a heavy chain variable region and/or a light chain variable region of any one of SEQ ID NOS: 01-30, as applicable, expressing at least one nucleic acid sequence in the vector to create at least one of a heavy chain, a light chain, or combinations thereof, and collecting a formed antibody or the antigen-binding fragment, thereof.

### Identification and Isolation of Antibodies

Antibodies of the disclosure may be isolated by screening combinatorial libraries for antibodies with the desired activity or activities. For example, a variety of methods are known in the art for generating phage display libraries and screening such libraries for antibodies possessing the desired binding characteristics (Hoogenboom et al. in Methods in Molecular Biology 178:1-37 (O'Brien et al., ed., Human Press, Totowa, N.J., 2001); McCafferty et al., Nature 348:552-554; Clackson et al., Nature 352: 624-628 (1991); Marks et al., J. Mol. Biol. 222: 581-597 (1992); Marks and Bradbury, in Methods in Molecular Biology 248:161-175 (Lo, ed., Human Press, Totowa, N.J., 2003); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132 (2004).

In certain phage display methods, repertoires of V_{H} and V_{L} genes are separately cloned by polymerase chain reaction (PCR) and recombined randomly in phage libraries, which can then be screened for antigen-binding phage. Phage typically display antibody fragments, either as single-chain Fv (scFv) fragments or as Fab fragments. Libraries from immunized sources provide high-affinity antibodies to the immunogen without the requirement of constructing hybridomas. Alternatively, the naive repertoire can be cloned (e.g., from human) to provide a single source of antibodies to a wide range of non-self without any immunization (Griffiths et al., EMBO J, 12: 725-734 (1993)). Finally, naive libraries can also be made synthetically by cloning unrearranged V-gene segments from stem cells, and using PCR primers containing random sequence to encode the highly variable CDR3 regions and to accomplish rearrangement in vitro (Hoogenboom and Winter, J. Mol. Biol., 227: 381-388 (1992)). Antibodies, including antigen-binding fragments, isolated from human antibody libraries are considered human antibodies.

### Methods of Treatment and Use

In a first aspect, the disclosure provides a method for treating a SARS-CoV-2 infection in a subject, the method comprising administering to said subject an effective amount of an isolated antibody, or an antigen-binding fragment thereof, that specifically binds to the spike protein of SARS-CoV-2, either alone, combined with one or more other antibodies, and/or as a part of a pharmaceutical composition.

In a second aspect, the disclosure provides a method for suppressing a SARS-CoV-2 infection in a subject, the method comprising administering to said subject an effective amount of an isolated antibody, or an antigen-binding fragment thereof, that specifically binds to the spike protein of SARS-CoV-2, either alone, combined with one or more other antibodies, and/or as a part of a pharmaceutical composition.

In a third aspect, the disclosure provides a method for preventing a SARS-CoV-2 infection in a subject, the method comprising administering to said subject an effective amount of an isolated antibody, or an antigen-binding fragment thereof, that specifically binds to the spike protein of SARS-CoV-2, either alone, combined with one or more other antibodies, and/or as a part of a pharmaceutical composition.

In a fourth aspect, the disclosure provides a method for treating, suppressing and/or preventing a disease or condition relating to a SARS-CoV-2 infection in a subject, the method comprising administering to said subject an effective amount of an isolated antibody, or an antigen-binding fragment thereof, that specifically binds to the spike protein of SARS-CoV-2, either alone, combined with one or more other antibodies, and/or as a part of a pharmaceutical composition. In certain embodiments of the fourth aspect, the disease or condition is Guillain-Barre Syndrome. In certain embodiments of the fourth aspect, the disease or condition is multisystem inflammatory syndrome, particularly when the subject is under the age of 25 years of age. In certain embodiments of the fourth aspect, the disease or condition is organ injury, such as, but not limited to, lung injury, liver injury, and/or heart injury. In certain embodiments of the fourth aspect, the disease or condition is acute respiratory distress syndrome. In certain embodiments of the fourth aspect, the disease or condition is increased inflammation resulting from an imbalance in the renin-angiotensin system (such as, but not limited to, excess production of angiotensin II and/or the decreased production of angiotensin 1-7).

In a fifth aspect, the disclosure provides a method of reducing or preventing cellular entry of SARS-CoV-2 in a subject, the method comprising administering to said subject an effective amount of an isolated antibody, or an antigen-binding fragment thereof, that specifically binds to the spike protein of SARS-CoV-2, or a combination of the foregoing, either alone, combined with one or more other antibodies, and/or as a part of a pharmaceutical composition.

In a sixth aspect, the disclosure provides a method of reducing or preventing binding of SARS-CoV-2 to a cellular ACE2 in a subject, the method comprising administering to said subject an effective amount of an isolated antibody, or an antigen-binding fragment thereof, that specifically binds to the spike protein of SARS-CoV-2, either alone, combined with one or more other antibodies, and/or as a part of a pharmaceutical composition.

In a seventh aspect, the disclosure provides a method for reducing viral titer of a SARS-CoV-2 in a bodily fluid, tissue or cell of a subject, the method comprising administering to said subject an effective amount of an isolated antibody, or an antigen-binding fragment thereof, that specifically binds to the spike protein of SARS-CoV-2, either alone, combined with one or more other antibodies, and/or as a part of a pharmaceutical composition. In certain embodiments, the transmission of the SARS-CoV-2 (for example, from a subject infected with SARS-CoV-2 to a subject that is not yet infected) is reduced as a result of a reduced viral titer.

In an eighth aspect, the disclosure provides a method for reducing or preventing the transmission of a SARS-CoV-2 infection from a first subject to a second subject, the method comprising administering to said first subject an effective amount of an isolated antibody, or an antigen-binding fragment thereof, that specifically binds to the spike protein of SARS-CoV-2, either alone, combined with one or more other antibodies, and/or as a part of a pharmaceutical composition. In certain embodiments of the eighth aspect, such reduction or prevention is obtained, at least in part, by reducing the cellular entry of a SARS-CoV-2 in the first subject. In certain embodiments, administration to the first subject occurs before the first subject has been infected with SARS-CoV-2, after the first subject has been infected with the SARS-CoV-2, or after the first subject has been infected with the SARS-CoV-2 and before the SARS-CoV-2 infection can be detected.

In a ninth aspect, the disclosure provides a method for reducing or preventing the transmission of a SARS-CoV-2 infection from a first subject to a second subject, the method comprising administering to the second subject an effective amount of an isolated antibody, or an antigen-binding fragment thereof, that specifically binds to the spike protein of SARS-CoV-2, either alone, combined with one or more other antibodies, and/or as a part of a pharmaceutical composition. In certain embodiments of the ninth aspect, the second subject may be at risk for SARS-CoV-2 infection. In certain embodiments of the ninth aspect, such reduction or prevention is obtained, at least in part, by preventing or reducing SARS-CoV-2 cellular entry in the second subject. In certain embodiments of the ninth aspect, such reduction or prevention is obtained, at least in part, by preventing or suppressing a SARS-CoV-2 infection in the second subject. In certain embodiments of the ninth aspect, if a SARS-CoV-2 infection occurs in the second subject, it can be eliminated physiologically (for example, by the immune system) by the second subject, either with or without the administration of additional therapeutic compounds. In certain embodiments of the ninth aspect, administration to the second subject before the second subject has been infected with the SARS-CoV-2, after the second subject has been infected with the SARS-CoV-2, or after the second subject has been infected with the SARS-CoV-2 and before the SARS-CoV-2 infection can be detected.

In a tenth aspect, the disclosure provides a method of neutralizing a SARS-CoV-2 in a subject, the method comprising administering to said subject an effective amount of an isolated antibody, or an antigen-binding fragment thereof, that specifically binds to the spike protein of SARS-CoV-2, either alone, combined with one or more other antibodies, and/or as a part of a pharmaceutical composition.

In certain embodiments of the methods of the first to tenth aspects, the antibody, or an antigen-binding fragment thereof, binds to SARS-CoV-2 viral particles before they are able to interact with cellular ACE2, thereby reducing or preventing SARS-CoV-2 viral particles from entering the cell. In certain embodiments of the methods of the first to tenth aspects, the antibody, or an antigen-binding fragment thereof, reduces or prevents SARS-CoV-2 viral particles from binding to cellular ACE2. In certain embodiments of the methods of the first to tenth aspects, the antibody, or an antigen-binding fragment thereof, reduces cleavage of the SARS-CoV-2 spike protein. In certain embodiments of the methods of the first to tenth aspects, the antibody, or an antigen-binding fragment thereof, reduce binding of SARS-CoV-2 to a target cell. In certain embodiments of the methods of the first to tenth aspects, the antibody, or an antigen-binding fragment thereof, reduces cellular fusion between SARS-CoV-2 and a target cell. In certain embodiments of the methods of the first to tenth aspects, the antibody, or an antigen-binding fragment thereof, reduces release of infective SARS-CoV-2 from an infected cell. In certain embodiments of the methods of the first to tenth aspects, the antibody, or an antigen-binding fragment thereof, reduce infection of a target cell by SARS-CoV-2.

The methods of the first to tenth aspects may further comprise one or more of the steps: i) identifying a subject in need or treatment, prevention, suppression, reduction, or inhibition; and (ii) providing an antibody, or an antigen-binding fragment thereof, of the disclosure or a pharmaceutical composition comprising the foregoing.

In certain embodiments of the first to tenth aspects, the antibody or antibodies, or an antigen-binding fragment(s) thereof, is any one or more antibodies or antigen binding fragment(s) described herein, or a pharmaceutically acceptable form thereof.

In certain embodiments of the first to tenth aspects, the antibody, or an antigen-binding fragment thereof, comprises: (i) a heavy chain variable region comprising the amino acid sequence selected from the group consisting of SEQ ID NOS: 01, 03, 05, 07, 09, 11, 13, 15, 17, 19, 21, 23, 25, 27 and 29, and (ii) a light chain variable region comprising the amino acid sequence selected from the group consisting of SEQ ID NOS: 02, 04, 06, 08, 10, 12, 14, 16, 18, 20, 22, 24, 26, 28 and 30 or a pharmaceutically acceptable form of any of the foregoing, such as, but not limited to a pharmaceutically acceptable salt, solvate and/or hydrate.

In certain embodiments of the first to tenth aspects, the antibody, or an antigen-binding fragment thereof, comprises a mixture of any of the foregoing.

In certain embodiments of the first to tenth aspects, the antibody, or an antigen-binding fragment thereof, is present as a pharmaceutically acceptable salt.

In certain embodiments of the first to tenth aspects, the antibody, or an antigen-binding fragment thereof, binds an epitope within the RBD of the SARS-CoV-2 spike protein. In certain embodiments of the first to tenth aspects, the antibody, or an antigen-binding fragment thereof, binds an epitope within the RBM of the SARS-CoV-2 spike protein. In certain embodiments of the first to tenth aspects, the antibody, or an antigen-binding fragment thereof, binds an epitope within the S2 region of the SARS-CoV-2 spike protein.

In certain embodiments of the first to tenth aspects, the antibody, or an antigen-binding fragment thereof, reduces binding of SARS-CoV-2 to a target cell. In certain embodiments of the first to tenth aspects, the antibody, or an antigen-binding fragment thereof, reduces cellular fusion between SARS-CoV-2 and a target cell. In certain embodiments of the first to tenth aspects, the antibody, or an antigen-binding fragment thereof, reduces release of infective SARS-CoV-2 from an infected cell. In certain embodiments of the first to tenth aspects, the antibody, or an antigen-binding fragment thereof, reduces infection of a target cell by SARS-CoV-2.

In certain embodiments of the first to tenth aspects, the antibody, or an antigen-binding fragment thereof, is an antibody variant. In certain embodiments of the first to tenth aspects, the antibody, or an antigen-binding fragment thereof, is an antibody variant comprising one or more substitutions, deletions, and/or insertions relative to the parental antibody. In certain embodiments of the first to tenth aspects, the antibody, or an antigen-binding fragment thereof, is an antibody variant comprising one or more substitutions relative to the parental antibody.

In certain embodiments of the first to tenth aspects, the antibody, or an antigen-binding fragment thereof, is an antibody variant that has a longer half-life in vivo in a subject relative to the parental antibody, decreased immunogenicity in vivo in a subject relative to the parental antibody, or a combination of the foregoing.

In certain embodiments of the first to tenth aspects, the antibody, or an antigen-binding fragment thereof, comprises a variant Fc constant region. In certain embodiments of the first to tenth aspects, the antibody, or an antigen-binding fragment thereof, comprises a variant Fc constant region, wherein a protein moiety or non-protein moiety is linked to the Fc constant region. In certain embodiments of the first to tenth aspects, the antibody, or an antigen-binding fragment thereof, comprises a variant Fc constant region, wherein a water soluble polymer is linked to the Fc constant region. In certain embodiments of the first to tenth aspects, the antibody, or an antigen-binding fragment thereof, comprises a variant Fc constant region, wherein a polyethylene glycol polymer is linked to the Fc constant region. In certain embodiments of the first to tenth aspects, the antibody, or an antigen-binding fragment thereof, comprises a variant Fc constant region, wherein a polyoxazoline polymer is linked to the Fc constant region. In certain embodiments of the first to tenth aspects, the antibody, or an antigen-binding fragment thereof, comprises a variant Fc constant region, wherein the variant Fc constant region provides a longer half-life in vivo in a subject relative to the parental antibody, decreased immunogenicity in vivo in a subject relative to the parental antibody, or a combination of the foregoing.

In certain embodiments of the first to tenth aspects, the antibody, or an antigen-binding fragment thereof, is a human antibody. In certain embodiments of the first to tenth aspects, the antibody, or an antigen-binding fragment thereof, is a chimeric antibody. In certain embodiments of the first to tenth aspects, the antibody, or an antigen-binding fragment thereof, is a class-switched antibody.

In certain embodiments of the first to tenth aspects, the antibody, or an antigen-binding fragment thereof, is linked to a therapeutic agent. In certain embodiments of the first to tenth aspects, the antibody, or an antigen-binding fragment thereof, is linked to a detectable label. In certain embodiments of the first to tenth aspects, the antibody, or an antigen-binding fragment thereof, is linked to an enzyme. In certain embodiments of the first to tenth aspects, the antibody, or an antigen-binding fragment thereof, is linked to an enzyme inhibitor.

In certain embodiments of the first to tenth aspects, the antibody, or an antigen-binding fragment thereof, is an antigen-binding fragment. In certain embodiments of the first to tenth aspects, the antibody, or an antigen-binding fragment thereof, is an antigen-binding fragment selected from the groups consisting of: a Fab fragment, a F(ab)2 fragment, a Fab′ fragment, a Fd fragment, a Fv fragment, a disulfide-linked Fv (sdFv), a dAb fragment, an isolated CDR, a nanobody or single domain antibody, a portion of the V_{H} region containing a single variable domain and two constant domains, a diabody, a triabody, a tetrabody, scFv, scFv-FC, scFv-CH, scFab, and scFv-zipper.

In certain embodiments of the first to tenth aspects, the antibody, or an antigen-binding fragment thereof, the amino acid sequence of the antibody, or an antigen-binding fragment thereof, has at least 85% homology to the reference sequence, at least 90% homology to the reference sequence, at least 95% homology to the reference sequence, at least 96% homology to the reference sequence, at least 97% homology to the reference sequence, at least 98% homology to the reference sequence, or at least 99% homology to the reference sequence.

In certain embodiments of the first to tenth aspects, the antibody, or an antigen-binding fragment thereof, the amino acid sequence of the antibody, or an antigen-binding fragment thereof, is 100% homologous to the reference sequence across the CDRs and has at least 85% homology to the reference sequence across the FRs, at least 85% homology to the reference sequence across the FRs, at least 90% homology to the reference sequence across the FRs, at least 95% homology to the reference sequence across the FRs, at least 96% homology to the reference sequence across the FRs, at least 97% homology to the reference sequence across the FRs, at least 98% homology to the reference sequence across the FRs, or at least 99% homology to the reference sequence across the FRs.

In certain embodiments of the first to tenth aspects, the antibody, or an antigen-binding fragment thereof, is used in combination with other anti-viral agents as described herein, such as inhibitors of viral RNA polymerase activity and/or other serine and non-serine protease inhibitors.

In certain embodiments of the first to tenth aspects, a subject is infected with SARs-CoV-2 and by one or more additional viruses.

In certain embodiments of the first to tenth aspects, the antibody, or an antigen-binding fragment thereof, is administered in an effective amount. Suitable effective amounts are described in more detail herein. In certain embodiments of the first to tenth aspects, the administering step may comprise administering a single dose of an antibody, or an antigen-binding fragment thereof, according to a course of treatment (where the dose may contain an effective amount). In certain embodiments of the first to tenth aspects, the administering step may comprise administering more than one dose of the antibody, or an antigen-binding fragment thereof, according to a course of treatment (where one or more doses may contain an effective amount). In certain embodiments of the first to tenth aspects, the antibody, or an antigen-binding fragment thereof, in each dose administered during a course of treatment is not required to be the same. For example, in any of the methods of the first to tenth aspects, the administering step may comprise administering at least one loading dose and at least one maintenance dose during a course of treatment.

In certain embodiments of the first to tenth aspects, the administering step comprises administering a single dose or a plurality of doses comprising the antibody, or an antigen-binding fragment thereof, according to a course of treatment. In certain embodiments of the first to tenth aspects, the administering step comprises administering a dose or a plurality of doses comprising the antibody, or an antigen-binding fragment thereof, by intravenous administration according to a course of treatment. In certain embodiments of the first to tenth aspects, the administering step comprises administering a dose or a plurality of doses comprising the antibody, or an antigen-binding fragment thereof, by intranasal administration according to a course of treatment. In certain embodiments of the first to tenth aspects, the administering step comprises administering a dose or a plurality of doses comprising the antibody, or an antigen-binding fragment thereof, by pulmonary administration according to a course of treatment.

In certain embodiments of the first to tenth aspect, the subject is suffering from or suspected of suffering from a SARS-CoV-2 infection.

In certain embodiments of the first to tenth aspects, a pharmaceutical composition and/or medicaments comprising the antibody, or an antigen-binding fragment thereof, may be administered according to the methods described herein.

In certain embodiments of the first to tenth aspects, the subject is a mammal. In certain embodiments of the first to tenth aspects, the subject is a human.

In certain embodiments of the first to tenth aspects, the administering step occurs before the subject has been infected with SARS-CoV-2 (i.e., the subject is at risk for infection), after the subject has been infected with SARS-CoV-2 (but before an infection can be detected), or after a subject has been infected with SARS-CoV-2 and the infection can be detected.

In certain embodiments of the first to tenth aspects, the antibody, the subject is a healthcare worker, a first responder (for example, a policeman or a fireman), or a member of the military as such individuals may be required to undertake activities that place them at a higher risk of SARS-CoV-2 infection. In certain embodiments of the first to tenth aspects, the subject has travelled to a region where SARS-CoV-2 infections have been documented, the subject has had contact with a person who has travelled to a region where SARS-CoV-2 infections have been documented, the has had contact with a person who has a SARS-CoV-2 infection (including a SARS-CoV-2 infection that has not been detected) or is suspected of having a SARS-CoV-2 infection, the subject is a family member or acquaintance of a person who has a SARS-CoV-2 infection (including a CoV infection that has not been detected) or is at risk of having a SARS-CoV-2 infection, the subject is an infant or child (for example, a subject under the age of 18 years) who has a caregiver or parent who has a SARS-CoV-2 infection or is at risk of having a SARS-CoV-2 infection.

In certain embodiments of the first to tenth aspects, the subject may be suffering from pulmonary disease, cardiovascular disease, diabetes mellitus, bacterial superinfection, sepsis syndrome, hypertension, chronic lung disease (inclusive of asthma, chronic obstructive pulmonary disease, and emphysema), chronic renal disease, chronic liver disease, immunodeficiency, an immunocompromised condition, neurologic disorder, neurodevelopmental, or intellectual disability.

In certain embodiments of the first to tenth aspects, the antibody, or an antigen-binding fragment thereof, is administered parenterally, such as by intravenous administration, intramuscular administration, or subcutaneous administration, orally, or via the respiratory tract (for example, by pulmonary or intranasal administration). In certain embodiments of the first to tenth aspects, the antibody, or an antigen-binding fragment thereof, is administered intravenously. In certain aspects of the methods of the first to tenth aspects, the antibody, or an antigen-binding fragment thereof, is administered intramuscularly. In certain aspects of the methods of the first to tenth aspects, the antibody, or an antigen-binding fragment thereof, is administered intranasally.

### Additional Therapeutic Agents

In addition, the pharmaceutical compositions or methods described herein may further comprise one or more additional anti-viral agents in combination with an antibody of the disclosure. Examples of such anti-viral agents include, but are not limited to, those agent that inhibit replication of SARS-CoV-2, such by inhibition of a RNA polymerase activity of SARS-CoV-2, and protease inhibitors, including but not limited to, serine protease inhibitors (for example, inhibitors of TMPRSS2) and cysteine protease inhibitors. Representative agents include, but are not limited to, galidesivir, remdisivir, hydrochloroquine, chloroquine, irbesartan, toremifene, camphor, equiline, mesalazine, mercaptopurine, nafamostat, paraoxetine, sirolimus, carvedilol, dactinomycin, melatonin, quinacrine, eplerenone, enoclin, oxymethalone, ENU2000, azithromycin, lopinovir/ritonavir, umifenovir, cytovene, ganciclovir, trisodium phosphonoformate, ribavirin, interferon, d4T, ddI, AZT, amantadine, rimantadine, acyclovir, foscarnet, laninamivir, oseltamivir, zanamivir, favipiravir, baloxavir marboxil, and peramivir.

Other agents and synergistic combinations thereof are described in Bobrowski et al., bioRxiv (June 2020, https://doi.org/10.1101/2020.06.29.178889) and Ko, et al., bioRxiv (May 2020, https://doi.org/10.1101/2020.05.12.090035).

Compounds that relate to inhibition of influenza polymerase are described, for example, in U.S. Pat. Nos. 7,388,002; 7,560,434; and in U.S. patent application Ser. No. 12/440,697 (published as U.S. Patent Publication No. 20100129317); and 12/398,866 (published as U.S. Patent Publication No. 20090227524).

### Dosage and Administration

In accordance with the methods of the disclosure, the polypeptides of the disclosure are administered to the subject (or are contacted with cells of the subject) in an effective amount. In some embodiments, an effective amount decreases the viral titer of SARS-CoV-2 in the subject and/or limits or prevents an increase in the viral titer of SARS-CoV-2 viral particles in the subject. In some embodiments, an effective amount decreases viral entry of SARS-CoV-2 subject. In some embodiments, an effective amount reduces binding of SARS-CoV-2 to a target cell. In some embodiments, an effective amount reduces cellular fusion between SARS-CoV-2 and a target cell. In some embodiments, an effective amount reduces release of infective SARS-CoV-2 from an infected cell of the subject. In some embodiments, an effective amount reduces infection of a target cell by SARS-CoV-2.

In certain embodiments, the effective amount of an antibody of the disclosure ranges from about 0.01 mg/kg to about 100 mg/kg. For example, doses of about 0.05 mg/kg to about 10 mg/kg, about 0.05 mg/kg to about 5 mg/kg, about 0.05 mg/kg to about 4 mg/kg, about 0.05 mg/kg to about 2 mg/kg, or about 0.05 mg/kg to about 1 mg/kg.

In certain embodiments, the effective amount of an antibody of the disclosure ranges from: i) about 1 mg/kg to about 5 mg/kg; ii) about 1 mg/kg to about 4 mg/kg; iii) about 1 mg/kg to about 4 mg/kg; or iv) about 1 mg/kg to about 4 mg/kg.

In certain embodiments, the effective amount described herein are administered every day, every other day, every three days, every 4 days, every 5 days, every six days or every seven days. In certain embodiments, the effective amount described herein are administered in weekly intervals (such as every week, every 2 weeks, every three weeks, every 4 weeks, every 5 weeks, every 6 weeks, every 7 weeks, every 8 weeks, or longer). In certain embodiments, the effective amount described herein are administered in monthly intervals (such as every month, every 2 months, every three months, every 4 months, every 5 months, every 6 months, every 7 months, every 8 months, every 9 months, every 10 months, every 11 months, every 12 months, or longer).

In one embodiment, an effective amounts is administered every day during a course of treatment. The effective amount per day may be administered in a single dose or in more than 1 dose per day (such as two to three doses per day). In certain embodiments, the effective amount per day is administered as a single dose per day. In certain embodiments, the effective amount per day is administered in two doses each day (i.e., b.i.d.), wherein the amount of the antibody in each dose need not be the same.

In one embodiment, the effective amount described above is administered every other day, every three days, every 4 days, every 5 days, every six days, or every seven days during a course of treatment. The effective amount in the dosing schedules may be administered in a single dose or in more than 1 dose (such as two to three doses). In certain embodiments, the effective amount in the dosing schedules is administered as a single dose on the day of administration. In certain embodiments, the effective amount in the dosing schedules is administered in two doses on the day of administration, wherein the amount of the antibody in each dose need not be the same.

In one embodiment, the effective amounts described above are administered in weekly intervals (such as every week, every 2 weeks, every three weeks, every 4 weeks, every 5 weeks, every 6 weeks, every 7 weeks, every 8 weeks, or longer), during a course of treatment. The effective amount per weekly interval may be administered in a single dose or in more than 1 dose. In certain embodiments, the effective amount per week is administered as a single dose on the day of administration. In certain embodiments, the effective amount per week is administered in two doses on the day of administration, wherein the amount of the antibody in each dose need not be the same.

In one embodiment, the effective amounts described above are administered in monthly intervals (such as every month, every 2 months, every three months, every 4 months, every 5 months, every 6 months, every 7 months, every 8 months, every 9 months, every 10 months, every 11 months, every 12 months, or longer), during a course of treatment. The effective amount per monthly interval may be administered in a single dose or in more than 1 dose. In certain embodiments, the effective amount per month is administered as a single dose on the day of administration. In certain embodiments, the effective amount per month is administered in two doses on the day of administration, wherein the amount of the antibody in each dose need not be the same.

As used herein, the term “dose” refers to an amount of an antibody of the disclosure administered at a given time point. For example, if a course of treatment for an antibody of the disclosure is b.i.d (2 times/administrations per day) for 7 days, each administrations on each of days 1-7 would comprise administering a dose (for 2 doses each day).

When 2 or more doses are administered on a given day, each dose administered may contain the same amount of an antibody of the disclosure or one or more of doses administered may contain a greater or lesser amount of an antibody of the disclosure as compared to another dose administered on that day. For example, if a course of treatment for an antibody of the disclosure calls for monthly administration, with the effective amount administered b.i.d at each monthly administration, the first dose administered may contain a first amount (i.e., 10 mg/kg) and the second dose administered may contain a second amount (i.e., 5 mg/kg). As another example, if a course of treatment for an antibody of the disclosure is b.i.d for 7 days, the first dose administered on day 1 may contain a first amount (i.e., 10 mg/kg), the second dose administered on day 1 may contain a second amount (i.e., 5 mg/kg), the two doses administered on each of days 2-4 may contain the second amount, and the two doses administered on each of days 5-7 may contain a third amount (i.e., 2 mg/kg).

Furthermore, for a given course of treatment, the administration schedule for the effective amount is not required to be the same. For example, a course of treatment may call for an effective amount to be delivered monthly (either as a single dose or multiple doses) for the first 6 months, and then every three months (either as a single dose or multiple doses) for the next 12 months.

Still further, for a given course of treatment, the effective amount delivered at each administration during a course of treatment is not required to be the same. For example, a course of treatment may call for an effective amount to be delivered monthly (either as a single dose or multiple doses) for the first 6 months, and then every two months (either as a single dose or multiple doses) for the next 6 months, wherein the effective amount for the first monthly administration is 10 mg/kg and the effective amount for the remaining monthly administration and the bi-monthly administrations is 2.5 mg/kg.

Any given dose may be delivered in a single unit dose form or more than one unit dose form. For example, a dose when given by IV administration may be provided as a single IV infusion (i.e., a single 20 mg/kg IV infusion) or as two or more IV infusions administered one after the other and be considered a single dose (i.e., two 10 mg/kg IV infusions). A dose may be further divided into a sub-dose. A sub-dose might be, for example, a number of discrete loosely spaced administrations, such as multiple inhalations from an inhaler, by application of a plurality of drops into the eye, or multiple tablets for oral administration.

In certain embodiments, only one dose of an antibody of the disclosure is administered during a course of treatment and no further doses are administered. Therefore, in the methods described herein the methods may comprise the administration of a single dose of an effective amount of an antibody of the disclosure during the entire course of treatment. In certain embodiments, more than one dose of an antibody of the disclosure is administered during a course of treatment. Therefore, the methods may comprise the administration of multiple doses of an antibody of the disclosure during the course of treatment. In certain embodiments, the course of treatment may range from 2 days to 1 month, from 2 days to 3 weeks, from 2 days to 2 weeks, or from 2 days to 1 week. In certain embodiments, the course of treatment may range from 1 month to 6 months, from 1 month to 12 months, from 1 month to 24 months, or from 1 month to 36 months. In certain embodiments, the course of treatment may range from 2 days to 6 days, from 2 days to 5 days, from 2 days to 4 days, or from 2 days to 3 days. In certain embodiments, a dose is delivered at least 1 time per day (i.e., 1 to 3 times) during the course of treatment. In certain embodiments, the course of treatment is continuous. In certain embodiments, a dose is not administered every day during the course of treatment (for example, a dose is be administered at least 1 time per day every other day during the course of treatment). Furthermore, the amount of an antibody of the disclosure in each dose need not be the same as discussed above. In certain embodiments, of the foregoing, one or more doses, preferably all of the doses, contain an effective amount of an antibody of the disclosure.

In certain embodiments, a course of treatment may comprise administering at least one dose as a loading dose and at least one dose as a maintenance dose, wherein the loading dose contains a greater amount of an antibody of the disclosure as compared to the maintenance dose (such as, but not limited to, 2 to 10 times higher). In one aspect of this embodiment, the loading dose is administered initially, followed by administration of one or more maintenance doses through the remaining course of treatment. For example, for a course of treatment that is b.i.d. for 7 days, a loading dose of 10 mg/kg may be administered as the first dose on day 1, followed by maintenance doses of 2 mg/kg for the remainder of the course of treatment. As a further example, for a course of treatment that is b.i.d. for 7 days, a loading dose of 20 mg/kg may be administered as the first dose on day 1, followed by maintenance doses of 5 mg/kg as the second dose on day 1 and each dose on days 2-4, followed by maintenance doses of 2 mg/kg for the remainder of the course of treatment.

Furthermore, a loading dose may be given as a dose that is not the first dose administered during a course of treatment. For example, a loading dose may be administered as the first dose on day 1 and as a dose on one or more additional days (for example, day 4). For example, for a course of treatment that is b.i.d. for 7 days, a loading dose of 10 mg/kg may be administered as the first dose on day 1, followed by a maintenance dose of 2 mg/kg as the second dose on day 1 and each dose on days 2-3, followed by a loading dose of 10 mg/kg as the first dose on day 4, followed by a maintenance dose of 2 mg/kg for the remainder of the course of treatment. When more than one loading dose is administered during a course of treatment, the loading dose may be the same (i.e., 10 mg/kg) or different (i.e., 20 mg/kg for the first loading dose and 10 mg/kg for each other loading dose).

In certain embodiments, the loading dose comprises 2 to 15 times more of an antibody of the disclosure as compared to a maintenance dose administered during the same course of treatment. In certain embodiments, the loading dose comprises 2 to 10 times more of an antibody of the disclosure as compared to a maintenance dose administered during the same course of treatment. In certain embodiments, the loading dose comprises 2 to 5 times more of an antibody of the disclosure as compared to a maintenance dose administered during the same course of treatment.

In certain embodiments of loading and maintenance doses as discussed above, one or more of the loading and maintenance doses, preferably all of the loading and maintenance doses, contain an effective amount of an antibody of the disclosure. Furthermore, in certain embodiments of the loading and maintenance doses discussed above, administration of one or more loading and/or maintenance doses may comprise administering one or more sub-doses and/or administering one or more unit dose forms.

In certain embodiments, the course of treatment is initiated (i.e., the first dose administered) after a subject has been infected with SARS-CoV-2. In certain embodiments, the course of treatment is initiated any time after a subject has been infected with SARS-CoV-2. In certain embodiments, the course of treatment is initiated any time after a subject has been infected with SARS-CoV-2 and before an active SARS-CoV-2 infection can be detected (i.e., by laboratory diagnosis or other methods). In certain embodiments, the course of treatment is initiated any time during which a subject has an active SARS-CoV-2 infection (i.e., by laboratory diagnosis or other methods). In certain embodiments, the course of treatment is initiated 1-5 days after a subject has been infected with SARS-CoV-2. In certain embodiments, the course of treatment is initiated 5-10 days after a subject has been infected with SARS-CoV-2.

In certain embodiments, the course of treatment is initiated before a subject is infected with SARS-CoV-2 (i.e., a prophylactic administration). For example, if a subject is planning to travel to a region where SARS-CoV-2 infection has been reported or believes he/she may be exposed to SARS-CoV-2, the subject may undergo a course of treatment with an antibody of the disclosure prior to travel to the region or prior to potential exposure. Furthermore, a subject may be someone that is not initially exposed to SARS-CoV-2 infection from a non-human vector source. For example, the spouse or partner of someone who has been exposed to SARS-CoV-2 or who is at risk for exposure to SARS-CoV-2 (for example, by traveling to an area where SARS-CoV-2 infection has been reported) may undergo a course of treatment with an antibody of the disclosure as well. Such a prophylactic use of the antibodies of the disclosure are beneficial not only to protect the subject that is administered an antibody of the disclosure, but also in protecting those the subject comes into contact with (for example, family members and co-workers).

In any of the foregoing embodiments, the dose may comprise an antibody of the disclosure alone or an antibody of the disclosure in a pharmaceutical composition. In any of the foregoing embodiments, each dose is delivered by parenteral administration. In any of the foregoing embodiments, each dose is delivered by IV administration. In any of the foregoing embodiments, each dose is delivered by IM administration. In any of the foregoing embodiments, each dose is delivered by subcutaneous administration. In any of the foregoing embodiments, each dose is delivered by via the pulmonary route. In any of the foregoing embodiments, each dose is delivered by pulmonary administration. In any of the foregoing embodiments, each dose is delivered by intranasal administration.

In any of the foregoing embodiments, each dose contains an amount of an antibody of the disclosure in a pharmaceutically acceptable form, such as a pharmaceutically acceptable salt.

### Pharmaceutical Compositions

The antibodies of the disclosure may be formulated into pharmaceutical compositions for administration to subjects in a pharmaceutically acceptable form suitable for administration in vivo. The disclosure provides a pharmaceutical composition comprising antibodies of the disclosure in combination with a pharmaceutically acceptable carrier and/or excipient. Often, the pharmaceutically acceptable carrier and/or excipient is chemically inert toward the active compounds and is non-toxic under the conditions of use. The pharmaceutically-acceptable carrier and/or excipient employed herein may be selected from various organic or inorganic materials that are used as materials for pharmaceutical compositions and which are incorporated as analgesic agents, buffers, wetting agents, emulsifying agents, pH adjusting agents, binders, disintegrants, diluents, emulsifiers, extenders, glidants, solubilizers, stabilizers, suspending agents, tonicity agents, vehicles, viscosity-increasing agents, antioxidants, colorants, flavor-improving agents, preservatives, and sweeteners.

In one embodiment, the disclosure provides a pharmaceutical composition comprising an antibody of the disclosure, wherein the pharmaceutical composition comprises 1 to 6,000 mg of the antibody. For example, the pharmaceutical composition may contain 1 to 2,000 mg, 1 to 600 mg, 1 to 500 mg, 1 to 400 mg, 1 to 300 mg, 1 to 200 mg, or 1 to 100 mg of the antibody. In one embodiment, the antigen of the disclosure is present in the pharmaceutical composition at a concentration of at least 1 mg/mL, 5 mg/mL, 10 mg/mL, 50 mg/mL, 100 mg/mL, 150 mg/mL, 200 mg/mL, 300 mg/mL, 400 mg/ml, or 500 mg/ml (with the concentration in each of the foregoing being less than 750 mg/ml).

The amount of an antibody of the disclosure in a pharmaceutical composition may very as is known in the art. Generally, the amount of an antibody of the disclosure will range from about 0.01% to about 99% by total weight of the pharmaceutical composition, preferably from about 0.1% to about 70%, from about 0.5% to 50%, or from about 1% to about 30%.

Examples of pharmaceutically acceptable carriers may include, for example, water or saline solution, polymers such as polyethylene glycol, carbohydrates and derivatives thereof, oils, fatty acids, or alcohols. In some embodiments, the pharmaceutically acceptable carrier is saline or water. In some embodiments, the pharmaceutically acceptable carrier is saline. In some embodiments, the pharmaceutically acceptable carrier is water or a saline solution.

Surfactants such as, but not limited to, detergents, are also suitable for use in the formulations. Specific examples of surfactants include polyvinylpyrrolidone, polyvinyl alcohols, copolymers of vinyl acetate and of vinylpyrrolidone, polyethylene glycols, benzyl alcohol, mannitol, glycerol, sorbitol or polyoxyethylenated esters of sorbitan; lecithin or sodium carboxymethylcellulose; or acrylic derivatives, such as methacrylates and others, anionic surfactants, such as alkaline stearates, in particular sodium, potassium or ammonium stearate; calcium stearate or triethanolamine stearate; alkyl sulfates, in particular sodium lauryl sufate and sodium cetyl sulfate; sodium dodecylbenzenesulphonate or sodium dioctyl sulphosuccinate; or fatty acids, in particular those derived from coconut oil, cationic surfactants, such as water-soluble quaternary ammonium salts of formula N⁺R′R″R′″R″″Y⁻, in which the R radicals are identical or different optionally hydroxylated hydrocarbon radicals and Y⁻is an anion of a strong acid, such as halide, sulfate and sulfonate anions; cetyltrimethylammonium bromide is one of the cationic surfactants which can be used, amine salts of formula N+R′R″R−′, in which the R radicals are identical or different optionally hydroxylated hydrocarbon radicals; octadecylamine hydrochloride is one of the cationic surfactants which can be used, non-ionic surfactants, such as optionally polyoxyethylenated esters of sorbitan, in particular Polysorbate 80, or polyoxyethylenated alkyl ethers; polyethylene glycol stearate, polyoxyethylenated derivatives of castor oil, polyglycerol esters, polyoxyethylenated fatty alcohols, polyoxyethylenated fatty acids or copolymers of ethylene oxide and of propylene oxide, amphoteric surfactants, such as substituted lauryl compounds of betaine,

When administered to a subject, the pharmaceutical composition is preferably sterile. The pharmaceutical compositions of the disclosure are prepared by methods well-known in the pharmaceutical arts. For example, the antibodies of the disclosure are brought into association with a carrier and/or excipient, as a suspension or solution. Optionally, one or more accessory ingredients (e.g., buffers, flavoring agents, surface active agents, and the like) also are added. The choice of carrier and/or excipient is determined by the solubility and chemical nature of the antibodies, chosen route of administration and standard pharmaceutical practice. In some embodiments, the pharmaceutical composition comprises an antibody of the disclosure and water. In some embodiments, the formulation comprises an antibody of the disclosure and saline.

For oral administration, a pharmaceutical composition of the disclosure may be presented as capsules, tablets, powders, granules, or as a suspension or solution. Capsule formulations may be gelatin, soft-gel or solid. Tablets and capsule formulations may further contain one or more adjuvants, binders, diluents, disintegrants, excipients, fillers, or lubricants, each of which are known in the art. Examples of such include carbohydrates such as lactose or sucrose, dibasic calcium phosphate anhydrous, corn starch, mannitol, xylitol, cellulose or derivatives thereof, microcrystalline cellulose, gelatin, stearates, silicon dioxide, talc, sodium starch glycolate, acacia, flavoring agents, preservatives, buffering agents, disintegrants, and colorants. Orally administered pharmaceutical compositions may contain one or more optional agents such as, but not limited to, sweetening agents such as fructose, aspartame or saccharin; flavoring agents such as peppermint, oil of wintergreen, or cherry; coloring agents; and preservative agents, to provide a pharmaceutically palatable preparation.

For parenteral administration the antibodies of the disclosure may be combined with a sterile aqueous solution that is isotonic with the blood of the subject. Such a formulation may be prepared by dissolving a solid active ingredient in water containing physiologically-compatible substances, such as sodium chloride, glycine and the like, and having a buffered pH compatible with physiological conditions, so as to produce an aqueous solution, then rendering said solution sterile. The formulation may be presented in unit dose form, such as sealed ampules or vials. The formulation may be delivered by any mode of injection, including, without limitation, epifascial, intracapsular, intracranial, intracutaneous, intrathecal, intramuscular, intraorbital, intraperitoneal, intraspinal, intrasternal, intravascular, intravenous, inhalation, intranasal, parenchymatous, subcutaneous, or sublingual or by way of catheter into the subject's body. A preferred mode of administration is intravenous, intramuscular, or intranasal.

Parenteral administration includes aqueous and non-aqueous based solutions. Examples of which include, for example, water, saline, aqueous sugar or sugar alcohol solutions, alcoholic (such as ethyl alcohol, isopropanol, glycols), ethers, oils, glycerides, fatty acids, and fatty acid esters. In some embodiments, water is used for parenteral administration. In some embodiments, saline is used for parenteral administration. Oils for parenteral injection include animal, vegetable, synthetic or petroleum based oils. Examples of sugars for solution include sucrose, lactose, dextrose, mannose, and the like. Examples of oils include mineral oil, petrolatum, soybean, corn, cottonseed, peanut, and the like. Examples of fatty acids and esters include oleic acid, myristic acid, stearic acid, isostearic acid, and esters thereof. In some embodiments, water is the excipient and/or carrier when the antibody of the disclosure is administered intravenously. In some embodiments, the excipient and/or carrier is a saline solution when the antibody of the disclosure is administered intravenously. In some embodiments, the excipient and/or carrier is a lactated Ringer's solution when the antibody of the disclosure is administered intravenously. Aqueous dextrose and glycerol solutions may also be employed as an excipient and/or carrier when the antibody of the disclosure is administered intravenously.

The antibodies of the disclosure can be formulated into aerosol formulations to be administered via the respiratory tract (for example, pulmonary or nasal administration). These aerosol formulations can be placed into pressurized acceptable propellants, such as dichlorodifluoromethane, propane, and nitrogen. Such aerosol formulations may be administered by metered dose inhalers. They also may be formulated as pharmaceuticals for non-pressured preparations, such as in a nebulizer or an atomizer.

The antibodies of the disclosure, alone or in combination with suitable excipients and/or carriers, may be administered in an aqueous solution as a nasal or pulmonary spray and may be dispensed in spray form by a variety of methods known to those skilled in the art. The formulations may be presented in multi-dose containers, for example in the sealed dispensing system disclosed in U.S. Pat. No. 4,511,069. Additional aerosol delivery forms may include, e.g., compressed air-, jet-, ultrasonic-, and piezoelectric nebulizers, which deliver the active agent dissolved or suspended in a pharmaceutical solvent, e.g., water, ethanol, or a mixture thereof.

Nasal and pulmonary solutions of the disclosure typically comprise the drug or drug to be delivered, optionally formulated with a surface-active agent, such as a nonionic surfactant (e.g., polysorbate-80), and one or more buffers. In some embodiments of the present invention, the nasal spray solution further comprises a propellant. The pH of the nasal spray solution is optionally between about pH 3.0 and 6.0, preferably 4.5+−0.5. Suitable buffers for use within these compositions are as described above or as otherwise known in the art. Other components may be added to enhance or maintain chemical stability, including preservatives, surfactants, dispersants, or gases. Suitable preservatives include, but are not limited to, phenol, methyl paraben, paraben, m-cresol, thimerosal, chlorobutanol, benzylalkonimum chloride, and the like. Suitable surfactants include, but are not limited to, oleic acid, sorbitan trioleate, polysorbates, lecithin, phosphatidyl cholines, and various long chain diglycerides and phospholipids. Suitable dispersants include, but are not limited to, ethylenediaminetetraacetic acid, and the like. Suitable gases include, but are not limited to, nitrogen, helium, chlorofluorocarbons (CFCs), hydrofluorocarbons (HFCs), carbon dioxide, air, and the like.

Within alternate embodiments, nasal and pulmonary formulations are administered as dry powder formulations comprising the active agent in a dry, usually lyophilized, form of an appropriate particle size, or within an appropriate particle size range, for intranasal delivery. Minimum particle size appropriate for deposition within the nasal or pulmonary passages is often about 0.5 μm. mass median equivalent aerodynamic diameter (MMEAD), commonly about 1 μm MMEAD, and more typically about 2 μm MMEAD. Maximum particle size appropriate for deposition within the nasal passages is often about 10 μm MMEAD, commonly about 8 μm MMEAD, and more typically about 4 μm MMEAD. Intranasally and pulmonary respirable powders within these size ranges can be produced by a variety of conventional techniques, such as jet milling, spray drying, solvent precipitation, supercritical fluid condensation, and the like. These dry powders of appropriate MMEAD can be administered to a patient via a conventional dry powder inhaler (DPI), which relies on the patient's breath, upon pulmonary or nasal inhalation, to disperse the power into an aerosolized amount. Alternatively, the dry powder may be administered via air-assisted devices that use an external power source to disperse the powder into an aerosolized amount, e.g., a piston pump.

To formulate compositions for nasal or pulmonary delivery, the active agent can be combined with various pharmaceutically acceptable additives, as well as a base or carrier for dispersion of the active agent(s). Desired additives include, but are not limited to, pH control agents, such as arginine, sodium hydroxide, glycine, hydrochloric acid, citric acid, etc. In addition, local anesthetics (e.g., benzyl alcohol), isotonizing agents (e.g., sodium chloride, mannitol, sorbitol), adsorption inhibitors (e.g., Tween 80), solubility enhancing agents (e.g., cyclodextrins and derivatives thereof), stabilizers (e.g., serum albumin), and reducing agents (e.g., glutathione) can be included. When the composition for nasal or pulmonary delivery is a liquid, the tonicity of the formulation, as measured with reference to the tonicity of 0.9% (w/v) physiological saline solution taken as unity, is typically adjusted to a value at which no substantial, irreversible tissue damage will be induced in the nasal mucosa at the site of administration. Generally, the tonicity of the solution is adjusted to a value of about ⅓ to 3, more typically ½ to 2, and most often ¾ to 1.7.

The antibodies of the disclosure may be dispersed in a base or vehicle, which may comprise a hydrophilic compound having a capacity to disperse the active agent and any desired additives. The base may be selected from a wide range of suitable carriers, including but not limited to, copolymers of polycarboxylic acids or salts thereof, carboxylic anhydrides (e.g., maleic anhydride) with other monomers (e.g., methyl (meth)acrylate, acrylic acid, etc.), hydrophilic vinyl polymers such as polyvinyl acetate, polyvinyl alcohol, polyvinylpyrrolidone, cellulose derivatives such as hydroxymethylcellulose, hydroxypropylcellulose, etc., and natural polymers such as chitosan, collagen, sodium alginate, gelatin, hyaluronic acid, and nontoxic metal salts thereof. Often, a biodegradable polymer is selected as a base or carrier, for example, polylactic acid, poly(lactic acid-glycolic acid) copolymer, polyhydroxybutyric acid, poly(hydroxybutyric acid-glycolic acid) copolymer and mixtures thereof. Alternatively or additionally, synthetic fatty acid esters such as polyglycerin fatty acid esters, sucrose fatty acid esters, etc. can be employed as carriers. Hydrophilic polymers and other carriers can be used alone or in combination, and enhanced structural integrity can be imparted to the carrier by partial crystallization, ionic bonding, crosslinking and the like. The carrier can be provided in a variety of forms, including, fluid or viscous solutions, gels, pastes, powders, microspheres and films for direct application to the nasal mucosa. The use of a selected carrier in this context may result in promotion of absorption of the active agent.

In some embodiments, the antibodies of the disclosure are in unit dose form such as a tablet, capsule, infusion bag for intravenous administration, or single-dose vial. Suitable unit dose forms may contain and effective amount (including specific examples of an effective amount described herein), The effective amount may be determined and/or modified during clinical trials designed appropriately for each of the conditions for which administration of an antibody of the disclosure is indicated and will, of course, vary depending on the desired clinical endpoint.

The disclosure also provides articles of manufacture for treating and preventing disorders, such as a SARS-CoV-2 infection, in a subject. The articles of manufacture comprise an antibody of the disclosure or a pharmaceutical composition comprising an antibody of the disclosure, optionally further containing at least one additional antiviral compound, as described herein. The articles of manufacture may be packaged with indications for various disorders that the pharmaceutical compositions are capable of treating and/or preventing. For example, the articles of manufacture may comprise a unit dose of an antibody of the disclosure that is capable of treating or preventing a certain disorder, and an indication that the unit dose is capable of treating or preventing a certain disorder, for example a SARS-CoV-2 infection.

### Detection and Diagnosis

The antibodies of the disclosure may be used for detection and diagnosis of SARS-CoV-2. Detection may occur by any known means in the art, for example, by immunoassay, including ELISA. Other immunoassays include, but are not limited to, enzyme immune assays (EIA), ELISPOT (enzyme-linked immunospot), radioimmunoassays (RIAs), immunofluorescence, and other assays known in the art, including but not limited to Western Blot analysis and/or immunoprecipitation methods.

For example in an ELISA (either direct or sandwich), a buffered solution of an antigen or a sample containing an antigen, is added to a well of a microtiter plate. A solution of non-reacting protein is then added to the well to prevent non-specific binding. An antibody of the disclosure, or antigen-binding fragment thereof, is added. Such antibody or antigen binding fragment may be typically conjugated to a reporter molecule, such as, but not limited to, luciferase, horse-radish peroxidase, alkaline phosphatase, or β-D-galactosidase. If the antibody of the disclosure, or antigen-binding fragment thereof, is not conjugated to a reporter molecule, secondary antibody that recognized the antibody of the disclosure may be added that is conjugated to a reporter molecule. A substrate for the reporter molecule is then added, which leads to a detectable signal. ELISAs may be run in a qualitative or quantitative format. Qualitative results provide a simple positive or negative result (yes or no) for a sample. A competitive ELISA may also be used. In this format, an unlabeled antibody of the disclosure, or antigen-binding fragment thereof, (the primary antibody, which may be conjugated to a reporter molecule or unlabeled) is incubated in the presence of a sample containing the antigen, which mixture is then added to a microtiter plate which is coated with the same antigen (the reference antigen) antigen-coated well. The plate is washed so as to remove unbound antibodies. If the primary antibody is not conjugated to a reporter molecule, a secondary antibody conjugated to a reporter molecule that is specific to the primary antibody is added to generate the detectable signal. Depending on the amount of antigen in the sample, more or less primary antibody will be available to bind the reference antigen. This means the more antigen there is in the sample, the less reference antigen will be detected and the weaker the detectable signal. Labeled antigens rather than labeled antibodies may be used. In this format, the less antigen in the sample, the more labeled antigen is retained and the stronger a detectable signal results.

In RIAs, typically a known quantity of an antigen is linked to a radioactive tracer, such as, but not limited to, 1-125, which is then mixed with a known amount of antibody of the disclosure to bind to an antigen (such as SARS-CoV-2 spike protein). Subsequently, a sample containing unknown quantity of the antigen is added and as the concentration of unlabeled antigen is increased, the binding between the antibodies and the labeled standard is decreased, which is directly measurable by measuring radioactivity.

In one aspect, the present disclosure provides a method for the determination (such as an immunoassay determination) of SARS-CoV-2 in a patient, the method comprising: a) incubating a bodily sample from the patient with at least one isolated antibody, or an antigen-binding fragment thereof, disclosed herein and a detectable label, wherein the detectable label is present on the antibody, or antigen binding fragment thereof, or the detectable label is present on a binding partner for either the SARS-CoV-2 or the antibody, or the antigen binding fragment thereof, to form an immunological complex containing the determinable group; and b) determining the presence of the detectable label in the sample, wherein the presence of the detectable label indicates SARS-CoV-2 is present in the sample.

In one embodiment, such method may further comprise isolating the immunological complex from the sample and determining the presence of the detectable label in either in the isolated immunological complex or in the sample remaining.

In certain embodiments, the bodily sample is a serum sample, a blood sample, a plasma sample, a throat swab sample, a nasopharyngeal swab sample, a sputum sample, a fecal sample, a urine sample, a saliva sample, or a bronchoalveolar lavage fluid sample.

Any assay described herein may be used for diagnostic purposes. Therefore, the disclosure provides for methods of diagnosing SARS-CoV-2 infection in a subject using the antibodies of the disclosure. Such a diagnostic use may be directed to determining if a SARS-CoV-2 infection is present, monitoring recovery from a SARS-CoV-2 infection, evaluating the efficacy of a therapeutic treatment for treating a SARS-CoV-2 infection, and for other purposes known in the art.

### Kits

The disclosure also provides a kit for use in the methods described herein, the kit comprising an antibody of the disclosure, or a pharmaceutically acceptable form thereof, and at least one of the following: (i) at least one other therapeutic agent; (ii) a buffer; (iii) instructions for administering the antibody of the disclosure, or pharmaceutically acceptable form thereof to a subject to treat a SARS-CoV-2 infection in the subject or detect SARS-CoV-2 in a sample (such as a sample from a subject).

In one embodiment of the kits disclosed, the subject is a human. In one embodiment of the kits disclosed, the antibody is an antibody of the first to second embodiments, or any of the specific aspects of the first to second embodiments described above.

The disclosure is further described by the following non-limiting Examples.

### Examples

### Example 1: Design and Production of S2 Probes

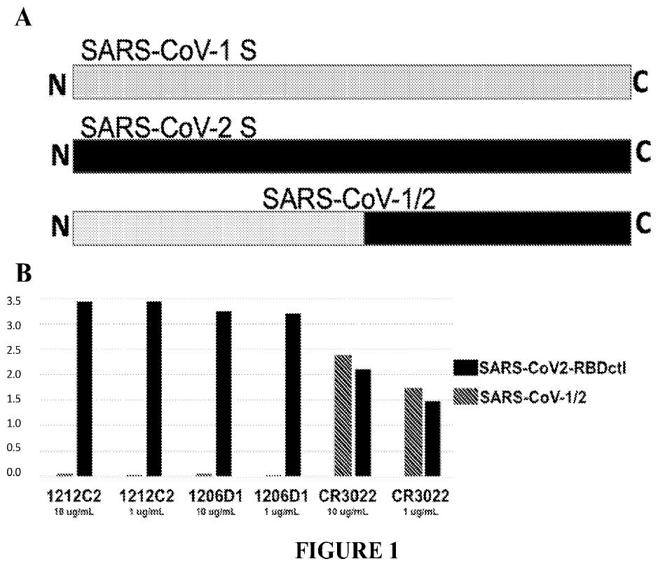

Proteins were designed to selectively identify B-cells that encode B-cell receptors that bind to the S2 region of the SARS-CoV-2 Spike. Two protein constructs Applicants made, called S2STBL and SARS-CoV-1/2 Spike chimera (SARS-CoV-1/2) (FIG. 1A). S2STBL consists of the Wuhan-1 Spike amino acid sequence 696-1211 followed sequentially by bacteriophage T4 fibritin protein residues 458-480, where F479 was changed to leucine, an 8-histidine affinity tag, and a C-terminal biotinylation tag. The SARS-CoV-2 S2 region contained structure-designed point mutations Q774C, L864C, S884C, and A893C to create additional disulfide bonds in the SARS-CoV-2 S2 domain to stabilize its pre-fusion conformation. Additional mutations K986P and V987P were also included in S2STBL. The cDNA encoding S2STBL was cloned into pMTV5His expression vector and transfected into insect cells. S2STBL protein expression was induced by copper sulfate and S2STBL was purified from the media by nickel affinity chromatography.

The SARS-CoV-1/2 chimera consists of SARS-CoV-1 residues 13-625 and SARS-CoV-2 residues 626-1211, where N627 is mutated to a serine, and K986 and V987 are mutated to proline. The chimeric spike sequence is followed by bacteriophage T4 fibritin protein residues 458-480, where F479 was changed to leucine, an 8-histidine affinity tag, and a C-terminal biotinylation tag. The cDNA encoding SARS-CoV-1/2 was cloned into pMTV5His expression vector and transfected into insect cells. SARS-CoV-1/2 protein expression was induced by copper sulfate. The resulting protein was purified from the media by nickel affinity chromatography. The chimeric protein was validated by ELISA, which confirmed the SARS-CoV-2 RBD-specific Abs (121C2 and 1206D1) do not recognize SARS-CoV-1/2, while CR3022, which recognizes the RBD of both SARS-CoV1 and SARS-CoV-2 also recognizes SARS-CoV-1/2 (FIG. 1B).

### Example 2: Identification, Purification, Sequencing, and Recombinant Production of SARS-CoV-2 Specific Antibodies

Peripheral blood samples were obtained from individuals following SARS-CoV-2/COVID-19 infection. Peripheral blood mononuclear cells (PBMC) were isolated by density gradient purification.

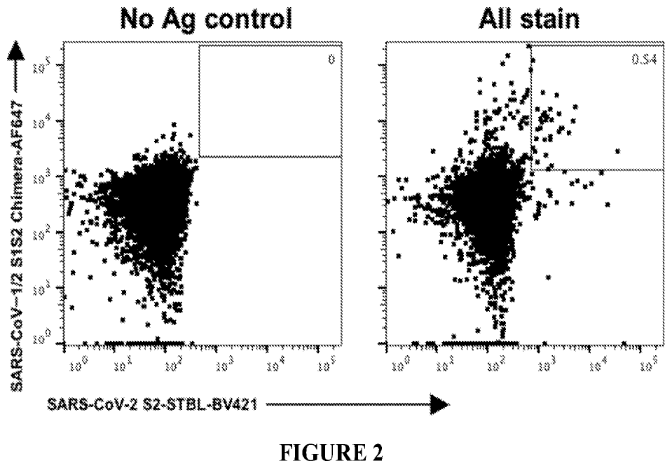

To obtain SAR-CoV-2 Spike S2 domain-specific B cells, PBMC were stained with fluorescently conjugated custom generated recombinant S2-STBL protein tetramer and SARS-CoV-1/2 S1S2 Chimera protein tetramer in addition to anti-CD19, anti-CD20, anti-CD27, anti-CD3, anti-CD14, anti-Annexin V at 4° C. for 60 min similar to as described in Kobie et al. Monoclon Antib Immunodiagn Immunother. 2015 Apr. 1; 34(2): 65-72, hereby incorporated by reference in its entirety. Single CD3-CD4-Annexin V-CD19+CD27+S2-STBL+ SARS-CoV-1/2 S1S2 Chimera+ cells (FIG. 2) were directly sorted using a FACSMelody (BD Biosciences) directly into 96-well PCR plates (Bio-Rad, Hercules, Calif.) containing 4 μL/well 0.5×PBS with 10 mM DTT (Invitrogen), and 8 U RiboLock (ThermoFisher) RNAse inhibitor. Plates were sealed with MICROSEAL F FILM (Bio-Rad) and immediately frozen at −80° C. until used for RT-PCR.

cDNA was synthesized and semi-nested RT-PCR for IgH, Igλ, and IgκV gene transcripts was performed as described in Kobie et al. IgG1 expression vector cloning and transfection of human HEK293T cells (ATCC, Manassas, Va.) were performed as previously described in Kobie et al. and Tiller et al., J Immunol Methods. 2008 Jan. 1; 329(1-2): 112-124, hereby incorporated by reference in its entirety. PCR products were sequenced at Genewiz Sequences and analyzed by IgBlast and IMGT/V-QUEST to identify germline V(D)J gene segments with highest identity and determine sequence properties (Table 2 below). IgG was purified from culture supermatant using MAGNA PROTEIN G beads (Promega, Madison, Wis.).

TABLE 2

| | | | | | % | | | % |
|---|---|---|---|---|---|---|---|---|
| | Native | | | | mutation | | | mutation |
| mAb | Isotype | VH | DH | JH | AA | VL | JL | AA |
| 1232D5 | IgG1 | IGHV3-30 | IGHD6-19 | IGHJ4 | 5.2 | IGLV1-44 | IGLJ3 | 4.1 |
| 1235C10 | IgG1 | IGHV3-49 | IGHD3-10 | IGHJ4 | 3.0 | IGK4-1 | IGKJ1 | 5.0 |
| 1242C6 | IgG1 | IGHV1-46 | IGHD1-26 | IGHJ4 | 16.7 | IGK3-20 | IGKJ2 | 12.5 |
| 1242D11 | IgA1 | IGHV3-7 | IGHD3-22 | IGHJ2 | 4.1 | IGK3-20 | IGKJ2 | 4.2 |
| 1242E6 | IgG1 | IGHV3-7 | IGHD3-9 | IGHJ5 | 1.0 | IGL2-14 | IGLJ1 | 6.1 |
| 1242F4 | IgG1 | IGHV1-46 | IGHD3-16 | IGHJ4 | 18.8 | IGK3-20 | IGKJ2 | 10.4 |
| 1242F11 | IgG1 | IGHV3-7 | IGHD2-2 | IGHJ6 | 7.1 | IGK3-20 | IGKJ3 | 2.1 |
| 1242G6 | IgG1 | IGHV3-30 | IGHD3-10 | IGHJ4 | 19.4 | IGK1-9 | IGKJ5 | 10.5 |
| 1246C2 | IgG3 | IGHV3-21 | IGHD1-26 | IGHJ2 | 14.3 | IGK3-11 | IGKJ4 | 9.5 |
| 1246H7 | IgM | IGHV4-39 | IGHD3-16 | IGHJ4 | 6.1 | IGL2-11 | IGLJ3 | 4.1 |
| 1249A8 | IgG1 | IGHV1-46 | IGHD1-26 | IGHJ4 | 16.7 | IGK3-20 | IGKJ2 | 13.5 |
| 1250D2 | IgG1 | IGHV1-46 | IGHD1-26 | IGHJ4 | 16.7 | IGK3-20 | IGKJ2 | 10.4 |
| 1250E10 | IgG1 | IGHV1-46 | IGHD1-26 | IGHJ5 | 20.8 | IGK3-30 | IGKJ2 | 16.7 |

### Example 3: Binding of SARS-CoV-2 Antibodies to SARS-CoV-2 Spike Proteins

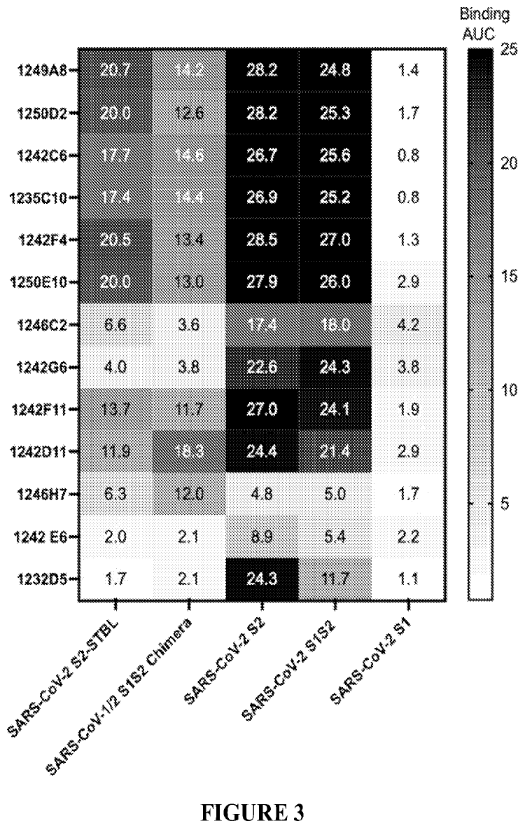

ELISA plates were coated with recombinant SARS-CoV-2 proteins, monoclonal antibodies were diluted in PBS, and binding detected with horseradish peroxidase-conjugated anti-human IgG (FIG. 3).

### Example 4: Neutralization of SARS-CoV-2 Virus by SARS-CoV-2 Antibodies

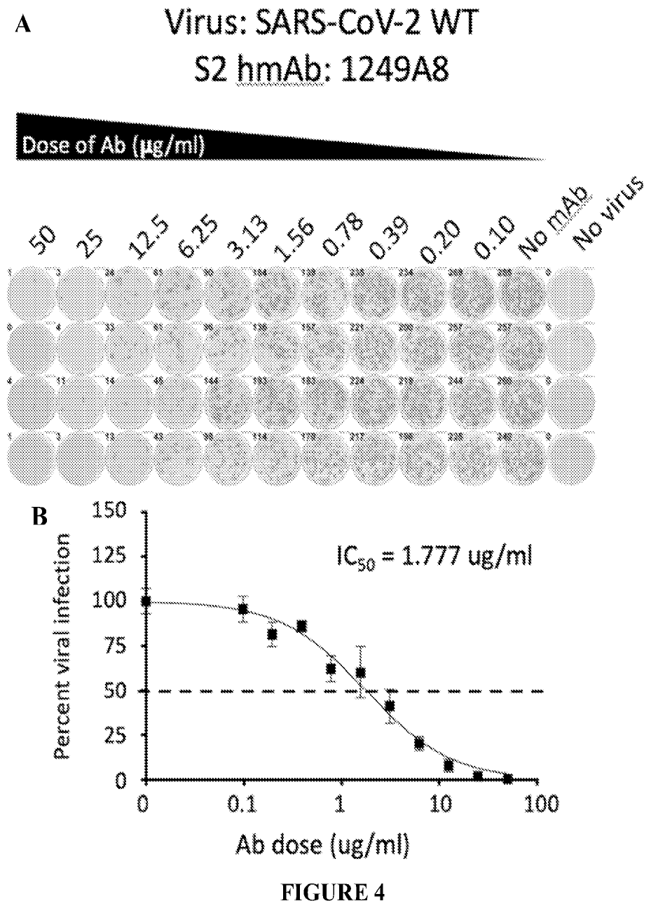

For live SARS-CoV-2 neutralization Vero HL cells (96-well plate format, 4×10⁴ cells/well, quadruplicate) were infected with 100 PFU/well of SARS-CoV-2 (FIG. 4A). After 1 h of viral adsorption, the infection media was changed with the 100 μl of media containing 100 Avicel and indicated concentrations (2-folds dilutions, starting 50,000 or 25,000 ng/ml of hmAbs or 1:100 dilution for human serum control). At 24 h p.i., infected cells were fixed with 10% neutral formalin for 24 h and were immunostained using anti-NP monoclonal 1C7C7 antibody. Virus neutralization was evaluated and quantified (Table 3, below) using ELISPOT, and the percentage of infectivity calculated using sigmoidal dose response curves (FIG. 4B). Mock-infected cells and viruses in the absence of hmAb (No hmAb) were used as internal controls.

mAbs were also tested using a SARS-CoV-2 Spike protein pseudotyped virus (PsV) containing the gene for firefly luciferase. Virus neutralization can be measured by the reduction of luciferase expression. VeroE6/TMPRSS2 cells were seeded at 2×10⁴ cells/well in opaque plates. The next day, PsV corresponding to 1-10×10⁶ luciferase units was mixed in Opti-MEM with dilutions of hmAbs and incubated at RT for 1 h. Media was removed from the cells and 100 μl/well of the hmAb/PsV mix was added in triplicates. After 1 h incubation at 37C and 5% CO₂, another 100 μL of media containing 2% FBS, was added, and cells were incubated for 24 more hr. After this time, luciferase activity was measured. Neutralization was calculated as the percent reduction of luciferase readings as compared to no-antibody-controls (FIG. 4).

TABLE 3

| | Neutralization IC₅₀ (μg/ml) | | |
|---|---|---|---|
| mAb | PsV D614G | PsV Beta | Live Virus WT |
| 1249A8 | 4.028 | 8.184 | 1.770 |
| 1250D2 | 7.592 | 8.392 | 2.361 |
| 1242C6 | 8.411 | 11.007 | 5.680 |
| 1235C10 | 8.753 | 8.815 | 10.450 |
| 1242F4 | 9.229 | 16.967 | 36.540 |
| 1250E10 | 3.64 | 4.168 | >50 |
| 1246C2 | 2.979 | 7.001 | >50 |
| 1242G6 | 23.681 | 25.608 | >50 |
| CV3-25 | 1.372 | >200 | 12.320 |
| 1212C2 | 0.003 | 0.003 | 0.003 |
| 1213H7 | 0.004 | 0.014 | ND |

### Example 5: Structure of the 1249A8/MERS-CoV SH Complex

To understand the structural basis for 1249A8's broad specificity and potency, the crystal structure of BNMAb 1249A8 Fab, in complex with a stem helix peptide (SHp) from MERS-CoV S, was determined at 2.1 Å resolution. The structure identifies the unique specificity determinants of the 1249A8-MERS-CoV SH interaction required for broad CoV binding and suggests 1249A8 inhibits membrane fusion by disrupting SH secondary structure rearrangements and sterically occluding SH-CH/HR1 interactions that are required for preF S to transition to its postF conformation.

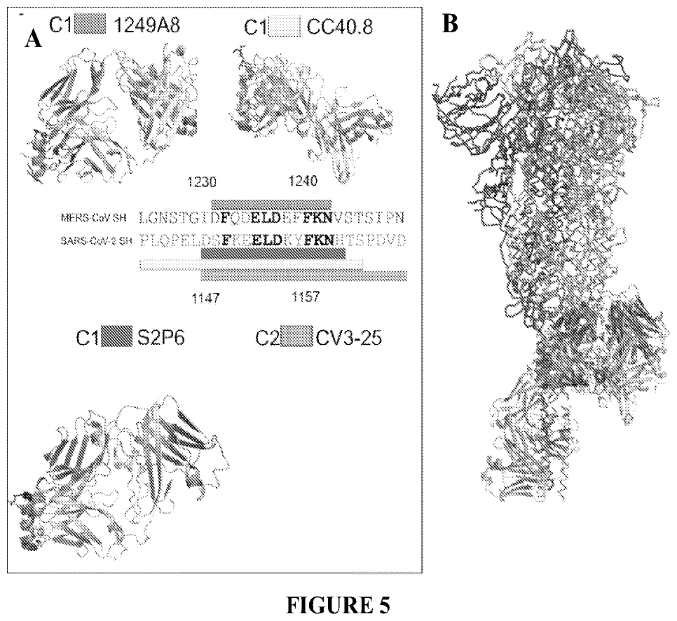

The crystal structure of 1249A8 Fab bound to a MERS-CoV S SHp, residues 1,223 to 1,245, was solved at 2.1 Å resolution (FIG. 5, Table 4). Of the 23 residues in the peptide, electron density was observed for 12 SHp residues (1,230-1,241) that form an amphipathic α-helix that binds in a groove between heavy and light chain CDRs of 1249A8. The MERS-CoV SHp sequence corresponds to SARS-CoV-2 residues 1,147-1,158. Three structures of human NAbs binding to the SARS-CoV-2 SH have been determined (FIG. 5). Fab-SHp structure comparisons reveal 1249A8, S2P6 and CC40.8 exhibit similar, but distinct, epitopes, defined as class-1 (C1) NAbs, that bind predominantly to the hydrophobic surface of the SH. Based on the cryo-EM structure of SARS-CoV-2 S, derived from the native S sequence, the C1 NAb epitope is buried in the center of the trimeric SARS-CoV-2 pre-fusion S coiled-coil (CC) domain (FIG. 5B). In contrast, CV3-25, defined as a class-2 (C2) NAb, targets exposed hydrophilic residues on SH and exhibits a distinct binding orientation, relative to the other NAbs (FIG. 5B). The C1 NAbs bind to SH using CDRs from their H and L chains, while the C2 CV3-25 binds almost exclusively (95%) through H chain CDR residues.

Superposition of 1249A8, and other C1 NAbs, onto the SH region of the preF S (pdbid 6xra) results in numerous steric clashes between S and Fab, suggesting that distortion/disruption of the trimeric CC region of S is required for NAb binding (FIG. 5B). Cryo-EM of the S2P6-SARS-CoV-2 S complex, a C1 SH-targeting NAb, clearly supports this hypothesis. In contrast, the surface exposed binding site of CV3-25 would induce limited distortion of the CC region (FIG. 5B). Structures of S from MERS-CoV, HKU1, SARS-CoV and SARS-CoV-2 (WA-1 strain) show the SH/CC region is disordered in 2P proline-stabilized S structures. Furthermore, tomograms of SARS-CoV-2 particles reveal the SH/CC region of S undergoes a variety of bending motions that are presumably important in the membrane fusion process. Together, these data suggest both C1 and C2 human NAb SH epitopes are at least temporarily accessible in the preF S conformation.

TABLE 4
Data Collection

| Beamline | SER-CAT 22-ID | |
|---|---|---|
| Wavelength, Å | 1.0 | |
| Space group | P2₁ | |
| Unit cell parameters (a, b, c, β) | 52.714, 56.308, 76.124, 91.191 | |
| Resolution, Å | 45.3-2.1 | |
| Number of Obs. | 86,813 | |
| Unique reflections | 26,008 | |
| Redundancyª | 3.3 | (3.0) |
| Completenessª | 98.6 | (93.9) |
| <I/sigI>ª | 5.0 | (1.9) |
| Rmrg (%)a | 0.119 | (0.436) |
| Rpim, (%)ª | 0.082 | (0.351) |
| CC1/2, (%)a | 0.967 | (93.9) |

Refinement Statistics

| | |
|---|---|
| Resolution (Å) | 43.8-2.1 |
| Reflections (work) | 25,899 |
| Reflections (test) | 1,410 |
| Rcryst/Rfree | 18.17/23.84 |
| No. of atoms: Protein | 3,433 |
| No. of atoms: Solvent | 282 |
| No. of atoms: Ions (Mg⁺², Cl⁻) | 11 |
| No. of atoms: Wilson B factor (Å²) | 29.5 |
| No. of atoms: Mean B factor (Å²) | 31.0 |

Model Geometry

| | |
|---|---|
| Bond lengths (Å) | 0.0087 |
| Bond angles (°) | 1.02 |
| Dihedrals (°) | 17.875 |
| Ramachandran plot: Favored (%) | 98.63 |
| Ramachandran plot: Outliers (%) | 0.23 |
| Ramachandran plot: PDB ID | processing |

^{a}Numbers in parentheses refer to highest resolution shell (2.16-2.10Å).
CC1/2 = Pearson correlation coefficient between two random half datasets.
${R}_{mrg} = {Σ}_{hkl} {Σ}_{i} \vert {I}_{i} ( hkl ) - \overline{I ( hkl )} \vert / {Σ}_{hkl} {Σ}_{i} {I}_{i} ( hkl ) .$ ${R}_{pim} = {{Σ}_{hkl} [ 1 / ( N - 1 ) ]}^{1 / 2} {Σ}_{i} \vert {I}_{i} ( hkl ) - \overline{I ( hkl )} \vert / {Σ}_{hkl} {Σ}_{i} {I}_{i} ( hkl )$ $\text{?} \text{indicates text missing or illegible when filed}$

Crystals of the 1249A8 Fab/MERS-CoV SHp complex (in 20 mM NaPO₄, pH 7,4, 100 mM NaCL, 10 mg/mL), were obtained from sitting drop vapor diffusion experiments, performed at 20C. All crystallization screens were performed using a Mosquito robot (SPT Labtech). Crystals were obtained in 200 nL drops consisting of 100 nL of complex with 100 nL well solution consisting of 0.2 M MgCl₂*6H₂O, 0.1M Tris, pH 8.5, 20% PEG-8000. Crystals, measuring 50 μm in each direction, were flash frozen in 25% glycerol 150 mM MgCl₂*6H₂O, 75 mM Tris, pH 8.5, 15% PEG-8000. Data were collected at the SER-CAT beamline 22-ID, Argonne National lab. The data were indexed, integrated, and scaled at 2.1 Å resolution using DIALS, in the CCP4 package. The structure was solved by molecular replacement using a model of 1249A8 Fab in Phenix. Model building was initially performed with ArpWarp and final modeling building and refinement was performed using Coot and Phenix, respectively.

### Example 6: 1249A8 Exhibits Higher Affinity for SH Peptide than Prefusion S

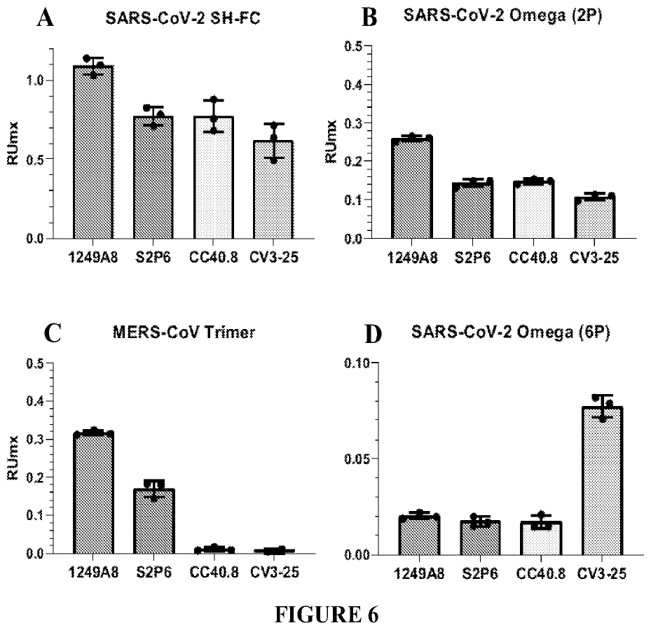

The affinity of 1249A8 to a SARS-CoV-2 SH peptide (residues 1,131-1,171) was defined to determine the role of S structure on 1249A8 binding (FIG. 6). 1249A8 bound preF S ˜8-fold weaker (KD=3.29 nM) than the SH peptide (KD=0.42 nM). 1249A8 exhibited the highest affinity for SH peptide, when compared to S2P6, CV3-25, and CC40.8, although the differences were small (˜2-fold), relative to NAb affinity differences observed when binding to S monomer (˜10-fold, Table 5). The higher binding affinity of the NAbs to the SH peptide may be at least partially due to avidity, as the SH peptide was expressed as an FC fusion protein. In addition to affinity, SH epitope accessibility in S trimers could influence NAb binding and function. To address this possibility, C1 and C2 NAb binding levels to various S proteins were determined (FIG. 6). Proteins evaluated in the study included the SH peptide, trimeric SARS-CoV-2 omicron VoC, expressed as a 2P or 6P stabilized S, and trimeric MERS-CoV S. Different omicron S proteins were evaluated since it has been reported that the 6P variant is more stable than the 2P form of S. The resulting surface plasmon resonance (SPR) data was consistent with previously determined affinity and specificity data of 1249A8, relative to other C1 and C2 NAbs. Specifically, 1249A8 exhibited the highest binding levels to the SH peptide, SARS-CoV-2 omicron-2P S, and MERS-CoV S. However, C2 NAb CV3-25 exhibited the highest binding to level to SARS-CoV-2-6P, while the three C1 NAbs all exhibited lower binding levels that were similar to each other. This data is consistent with the surface accessibility of the CV3-25 epitope, relative to the C1 NAbs (FIG. 5B).

TABLE 5

| | ka | kd | kD (nM) |
|---|---|---|---|
| SARS-CoV-2 S | | | |
| 1249A8 | 1.62 × 10⁵ | 5.31 × 10⁴ | 3.28 |
| S2P6 | 3.21 × 10⁵ | 1.03 × 10² | 32.00 |
| CV3-25 | 8.69 × 10⁴ | 1.15 × 10³ | 13.20 |
| CC40.8 | 1.46 × 10⁵ | 9.73 × 10⁴ | 6.69 |
| SH-FC | | | |
| 1249A8 | 6.18 × 10⁵ | 2.57 × 10⁴ | 0.42 |
| S2P6 | 4.63 × 10⁵ | 3.57 × 10⁴ | 0.77 |
| CV3-25 | 2.44 × 10⁵ | 2.55 × 10⁴ | 1.05 |
| CC40.8 | 3.90 × 10⁵ | 3.06 × 10⁴ | 0.78 |

SPR experiments were performed on a Biacore T200 (Cytiva) at 25° C. using a running buffer consisting of 10 mM HEPES, 150 mM NaCl, 0.0075% P20. Binding studies were performed by capturing the hmAbs to the chip surface of CM-5 chips using a human antibody capture kit (cytiva). Kinetic binding parameters were derived by SPR using SH-FC consisting of SH residues 1131-1171. SH-FC was injected over NAbs at four concentrations (25 nM, 6.125 nM, 1.56 nM, and 0.391 nM) with a contact time of 240 seconds and a 300 second dissociation time. All SPR experiments were double referenced (e.g., sensorgram data was subtracted from a control surface and from a buffer blank injection). The control surface for all experiments consisted of the capture antibody. Sensorgrams were globally fit to a 1:1 model, without a bulk shift correction, using Biacore T-200 evaluation software version 1.0. Fractional binding of SH-FC and CoV S proteins to NAbs was performed using the same injection parameters as for the kinetic experiments. S binding levels in RU, were converted to a fractional Rmax value by dividing RUobs by RUmax, where RUmax=NAb captured (RU)*[MW of S or SH-FC/150,000], where the MW of all trimeric S proteins was fixed at 525,000 and the MW of SH-FC was 54,000. S proteins used in the study were SARS-CoV-2 S GCN4-IZ-2P (10561-CV, R&D Systems), SARS-CoV-2 B.1.1.529 S GCN4-IZ-2P (11061-CV, R&D Systems), MERS-CoV S GCN4-IZ-2P (R&D Systems), and SARS-CoV-2 B1.1.529 S (40589-V08H26, Sino Biological). 1249A8 NAb was expressed in Expi293 cells using manufacturer's instructions and purified from the culture media using protein A affinity chromatography using MAbs select protein A resin (Cytiva). Fab was generated by papain (Sigma) digestion of 1249A8. Following digestion, the Fc was removed from the reaction using protein A resin. 1249A8 Fab was further purified by gel filtration chromatography. Purified 1249A8 Fab was incubated with a 5×molar excess of MERS-CoV SHp residues 1223-1245 for 30 minutes and the complex was concentrated to 10 mg/mL for crystallization.

### Example 7: 1249A8/MERS-CoV SHp Interface

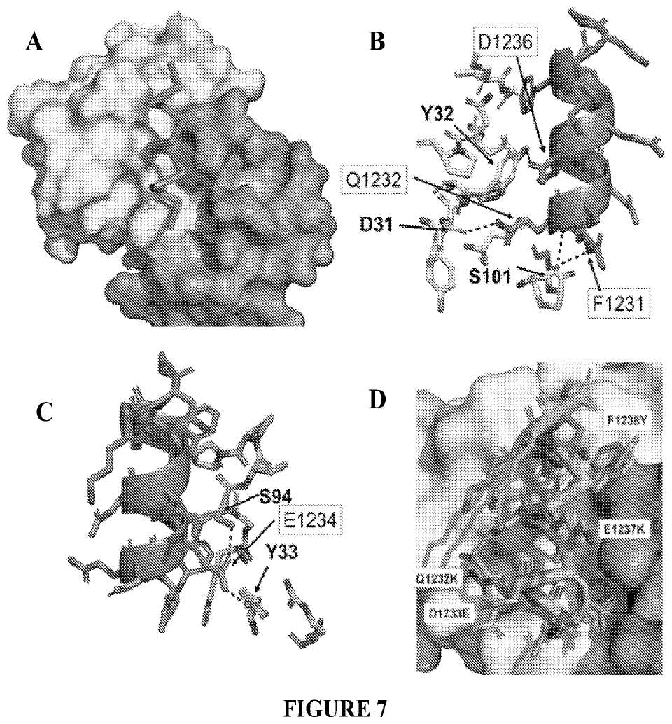

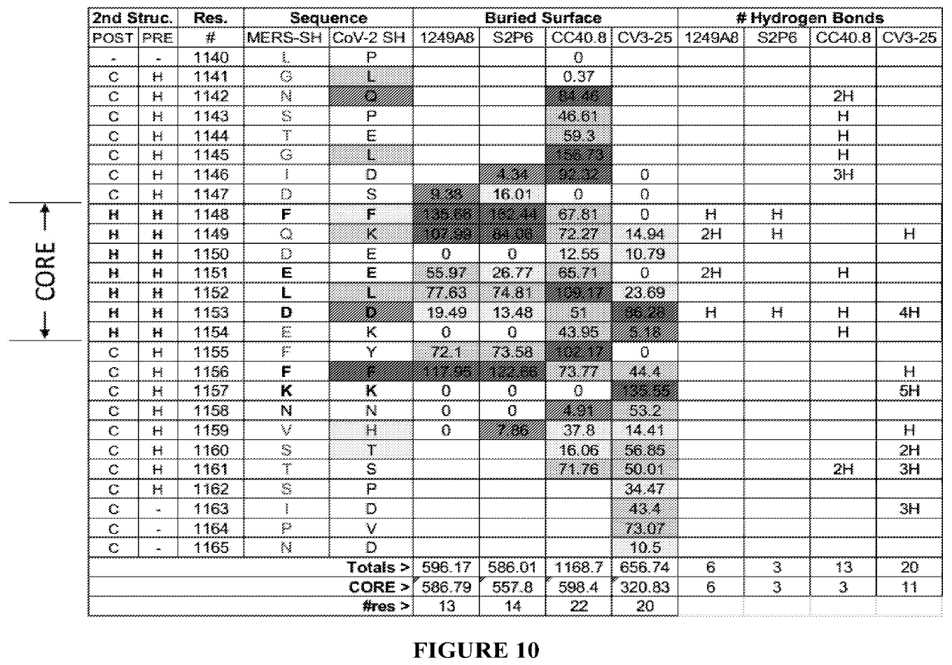

The MERS SHp α-helix buries 614 Å² of accessible surface area into 1249A8, which is distributed between heavy (368 Å²) and light (246 Å²) chain CDRs. Hydrophobic residues F1231 L1235, and F1239 bury the greatest amount of surface area into 1249A8 (FIG. 7, Table 6). Other SHp residues with hydrophobic and hydrophilic chemistries also bury significant amounts of surface area into 1249A8. However, these residues remain partially accessible to the solvent to accommodate alternative amino acid residues found in other CoV SH regions. A total of six hydrogen bonds are made between 1249A8 CDRs and the MERS-CoV SHp (FIGS. 7B, 7C, 10). Four are with heavy chain residues and two are with the light chain. Five of the six hydrogen bonds are formed with four N-terminal residues (1,231-1,234) of the epitope. Thus, while the 1249A8 epitope consists of residues 1,231-1,239, critical 1249A8 binding contacts are concentrated into the four-residue segment, residues 1,231-1,234. Two of the six hydrogen bonds are formed between the carbonyl oxygen of CDRH3 S101 and the backbone nitrogen atoms of SHp F1231 and Q1232. To form these hydrogen bonds, CDRH3 is positioned beneath the N-terminal end of the helix, effectively “capping” the SHp. While less pronounced, other C1 NAbs share a similar binding mode, suggesting C1 NAbs require the full length preF α-helical SH to unwind and/or bend to allow NAb binding.

TABLE 6

| Chain | # | MERS SHp Atom | Dist. [Å] | 1249A8 Atom |
|---|---|---|---|---|
| HC | 1 | PHE 1231 [N] | 2.80 | SER 101[O] |
| HC | 2 | GLN 1232[N] | 2.86 | SER 101[O] |
| HC | 3 | GLN 1232[NE2] | 2.71 | ASP 31[O] |
| HC | 4 | ASP 1236[OD1] | 2.50 | TYR 33[OH] |
| LC | 5 | GLU 1234[OE1] | 2.45 | SER 94[OG] |
| LC | 6 | GLU 1234[OE2] | 2.24 | TYR 33[OH] |

### Example 8: Molecular Basis for 1249A8's Broad Specificity

While all reported SH-targeting human NAbs bind to the SARS-CoV-2 SH, 1249A8 is the only NAb that also exhibits high affinity binding (KD=0.58 nM) to the MERS-CoV SH and neutralizing activity against MERS-CoV. The six amino acid differences between the MERS-CoV and SARS-CoV-2 SH epitopes have limited impact on 1249A8 interactions because they are either fully (D1229S, D1233E, E1237K, V1242H), or partially (Q1232K and F1238Y), accessible to solvent when bound to 1249A8. The Q^{MERs-Cov}1232K^{SARS-CoV-2} mutation is the only residue substitution that alters the hydrogen bonding found in the 1249A8-MERS-CoV SHp complex (FIG. 7D). 1249A8 CDRH1 accommodates both Q and K sidechains by forming a hydrogen bond between Q1232 and the main chain carbonyl of D31, while SARS-CoV-2 K1149 is positioned to interact with the negatively charged D31 sidechain (FIG. 7B). In both cases, the common aliphatic regions of MERS-CoV Q1232 and SARS-CoV-2 K1149 sidechains bury significant amounts of surface area into the 1249A8 interface to maintain high affinity binding. In contrast to 1249A8, other C1 NAbs exhibit weak (S2P6, KD 8.5 nM) or essentially no affinity (CV3-25 and CC40.8) for the MERS-CoV SH. Analysis of modeled S2P6/MERS-CoV SH and CC40.8/MERS-CoV SH complexes show replacement of K1149 with Q induces steric clashes within the S2P6 and CC40.8 binding sites, consistent with their low or absent affinity for MERS-CoV SH (FIG. 7D). Q1232 cannot be responsible for CV3-25's inability to bind to MERS-CoV SH, since the residue is not part of the binding epitope. However, 12 additional SARS-CoV-2 SH residues that form the CV3-25 binding epitope are different in the MERS-CoV SH (FIG. 5) and all could impact CV3-25/MERS-CoV SH interactions.

### Example 9: 1248A8 CDRH3 Mimics the SARS-CoV-2 S 3-Helix Motif

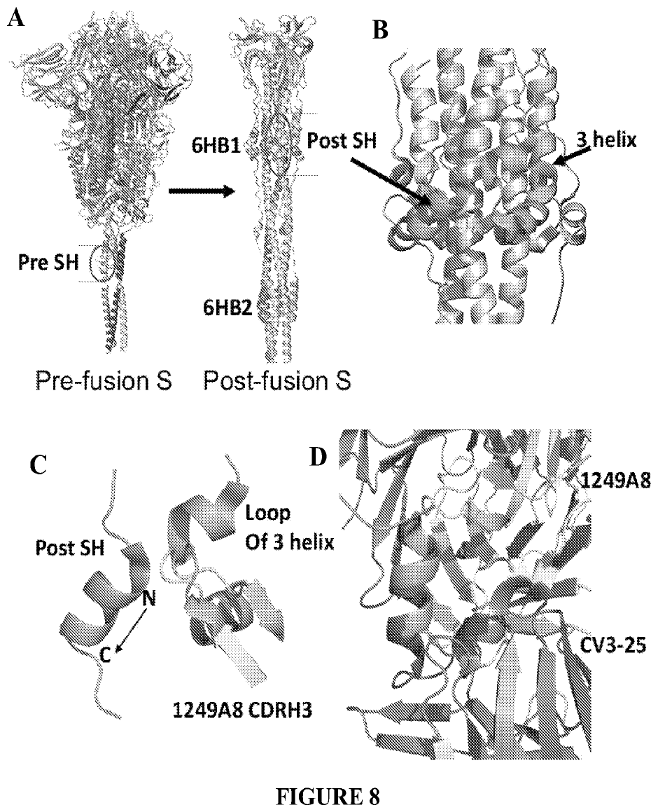

Based on the static preF SARS-CoV-2 S structure, optimal 1249A8 binding requires dissociation of trimeric S to remove Fab-S steric clashes and expose the 1249A8 epitope (FIG. 5B). In addition to trimer dissociation, 1249A8 cannot bind efficiently to the full-length α-helical SH (residues 1,141-1,161) due to steric clashes with CDRH3, which binds across the N-terminal end of SH residue F1148 (FIG. 7B). Thus, optimal 1249A8 binding requires the preF SH to unwind. SH unwinding occurs naturally as S transitions from its preF to postF conformation (FIG. 8A), changing from a 21-residue α-helix in preF S to a 7-residue core α-helix (core-SH, residues F1148-K1154) in the postF S (FIGS. 5, 8B). The core-SH is structurally conserved in preF and postF S conformations, exhibits high sequence conservation across CoV lineages, and contains the essential five 1249A8 binding residues required to form all six hydrogen bonds with 1249A8. This suggests 1249A8 is optimized to bind the postF SH. Superposition of 1249A8-SHp complex onto the core-SH in the postF S (6xra) reveals CDRH3 mimics the loop structure (residues 743-749, CGDSTEC) found in the 3-helix region (residues 737-769) of the postF S, where D745 caps the N-termini of the SH (FIG. 8C).

The data suggests 1249A8-mediated neutralization occurs by blocking 6HB1 assembly (FIG. 8A), and ultimately disrupting formation of the postF S structure required for membrane fusion. All C1 NAbs bind the N-terminal end of the postF core-SH and are hypothesized to neutralize virus by sterically interfering with 611131 formation as described for 1249A8. In contrast, C2 CV3-25 binds to the C-terminal end of the postF core SH on the accessible surface of the helix (FIG. 8D). As a result, CV3-25 does not block the SH/3-helix interaction and makes only minor steric clashes to prevent core-SH packing with CH/HR1 to form the 6HB1. In comparison to C1 NAbs, CV3-25 would be predicted to have limited ability to disrupt the formation of the postF if steric disruption of 6HB1 formation was the only mechanism used by SH-targeting NAbs.

### Example 10: 1249A8 Disrupts Post-Fusion SH Secondary Structure

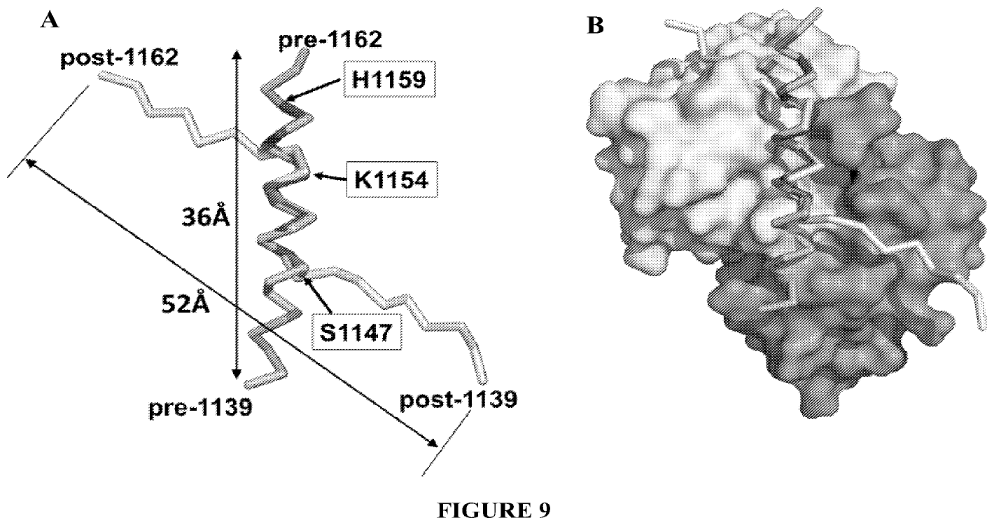

The SARS-CoV-2 SH makes an extensive transition from a preF 21-residue helix to a postF extended structure that retains a 7-residue α-helical core-SH, which changes the length of SH by 16A (FIG. 9). 1249A8 binding disrupts this SH transition by locking the SH in a pre-fusion α-helical conformation. 1249A8 forms all hydrogen bonds with the naturally occurring postF core-SH (F1148-K1154). However, the remainder of the 1249A8 epitope (Y1155-N1159) retains an α-helical conformation observed in the preF SH, while the same residues in postF SH adopt an extended conformation required for proper postF S refolding (FIG. 9B). Thus, 1249A8 binding disrupts SH secondary structure transitions required for SH and HR2 regions to properly pack against HR1 (FIGS. 8 and 9). Thus, 1249A8 is predicted to disrupt postF S 6HB1 and 6HB2 formation using steric and disrupted SH secondary structure mechanisms. All SH-targeting NAbs are expected to neutralize CoVs using similar mechanisms.

### Example 11: Identification and Isolation of S2-Specific Human B Cells

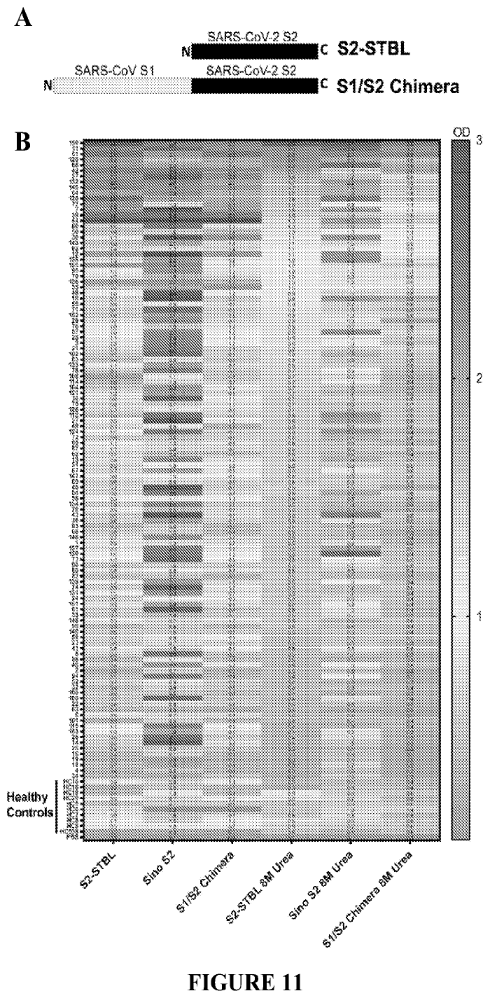

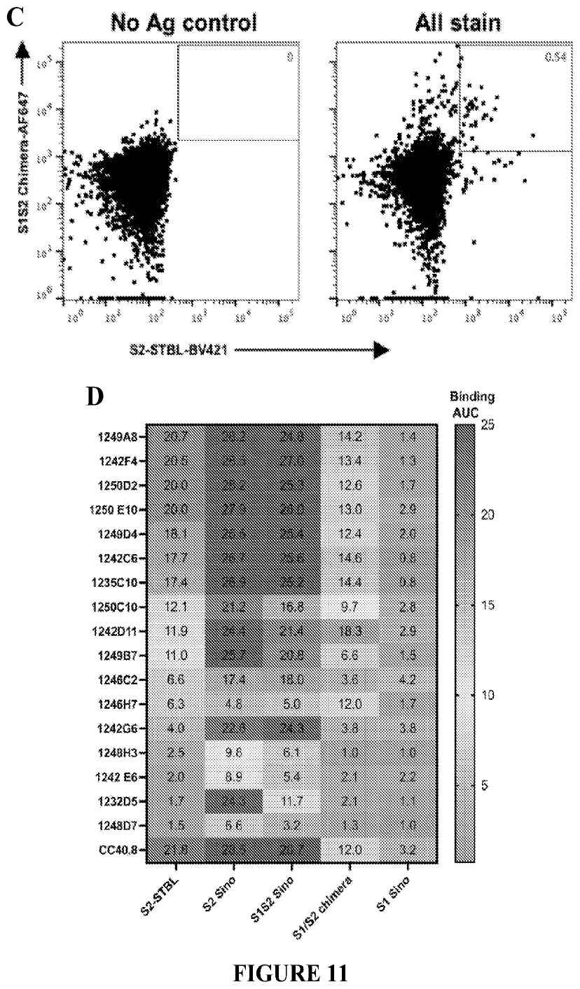

To identify SARS-CoV-2 S2-specific human B cells, two complementary recombinant proteins were designed and produced; a pre-fusion state stabilized SARS-CoV-2 (S2-STBL) and a SARS-CoV/SARS-CoV-2 full Spike chimera consisting of SARS-CoV S1 and SARS-CoV-2 S2 (SARS-CoV-1/2 S1S2) (FIG. 11A). Initial testing of plasma from COVID-19 convalescent patients was performed to identify those with high avidity IgG binding titers against S2 (FIG. 11B) from which to isolate S2-specific B cells. Using fluorescent S2-STBL and S1S2 chimera tetramers, peripheral blood memory B cells from several subjects were single-cell sorted by flow cytometry (FIG. 11C) and recombinant fully human IgG1 mAbs (hmAbs) were generated. Seventeen hmAbs with reactivity to SARS-CoV-2 S2 protein resulted (FIG. 11D). In general, most hmAbs bound commercial preparations of SARS-CoV-2 S, as well as S2-STBL and SARS-CoV-1/2, as shown in the plasma profiling. Binding to S2-STBL and SARS-CoV-1/2 S1S2 was more discriminating, as also evident in the plasma profiling. Differential binding profiles was observed for some plasma and mAbs to S2-STBL and S2 Sino, but the reason for this remains uncertain. The previously reported S2-specific hmAb CC40.8 was included as a positive control. Off-target binding to SARS-CoV-2 S1 was not evident.

For examples 11-16, peripheral blood was collected from adult convalescent patients approximately 1 month following PCR confirmed infection with SARS-CoV2. Peripheral blood mononuclear cells (PBMC) were isolated by density gradient centrifugation and cryopreserved. S2-STBL and a SARS-CoV-1/SARS-CoV-2 chimera (S1/S2) were used to generate tetramers for B-cell isolation. S2-STBL consists of the SARS-CoV-2 amino acid sequence (Wuhan-1) residues 696-1211, with mutations Q774C, L864C, S884C, A893C, K986P, and V987P. The prefusion S1S2 chimera contains SARS-CoV-1 S residues 13-634 (uniprot P59594) and SARS-CoV-2 Wuhan-1 (P0DTC2) S residues 635-1211. The S1S2 sequence contains mutations R682S, R683-A, K986P, and V987P. The C-termini of both proteins contain C-terminal T4 fibritin trimerization domains, his8 tags and biotinylation tags. The proteins were expressed in insect cells and purified by nickel affinity chromatography. The proteins were confirmed to have the correct sizes by Western blot and presence of SARSCoV-1 S1 by ELISA. The purified proteins were biotinylated using biotin ligase (BIRA, https://www.avidity.com/) and then used to form S2-STBL and S1S2 streptavidin tetramers for B cell isolation experiments. Cryopreserved cells were thawed and then stained for flow cytometry using anti-CD19-APC-Cy7 (SJ25C1, BD Biosciences), HIV gp140-AlexaFluor488, S2-STBL-BV421, S1/S2 chimera-AlexaFluor647, CD3-BV510 (OKT3, Biolegend), CD4-BV510 (H1I30, Biolegend), CD14-BV510 (63D3, Biolegend), CD27-PE (CLB-27/1,Life Technologies), Annexin V-PerCP-Cy5.5 (Biolegend), SA-BV421 (Biolegend), SA-AlexaFluor647 (Biolegend), and Live/Dead aqua (Molecular Probes).

For examples 11-16, single B cells were sorted using a FACSMelody (BD Biosciences) into 96-well PCR plates containing 4 μl of lysis buffer. Plates were immediately frozen at −80° C. after sorting until thawed for reverse transcription and nested PCR performed for IgH, Igλ, and Igκ variable gene transcripts. Paired heavy and light chain genes were cloned into IgG1 expression vectors and were transfected into HEK293T cells and culture supernatant was concentrated using 100,000 MWCO Amicon Ultra centrifugal filters (Millipore-Sigma, Cork, Ireland), and IgG captured and eluted from Magne Protein A beads (Promega, Madison, WI). Immunoglobulin sequences were analyzed by IgBlast (www.ncbi.nlm.nih.gov/igblast) and IMGT/V-QUEST (http://www.imgt.org/IMGT_vquest/vquest) to determine which sequences should lead to productive immunoglobulin, to identify the germline V(D)J gene segments with the highest identity, and to scrutinize sequence properties. CV3-25, S2P6, and CC40.8 were previously described and heavy and light chain variable regions synthesized by IDT based on reported sequences (GenBank: MW681575.1, GenBank: MW681603.1) and cloned into IgG1 expression vector for production in HEK293T cell. 1249A8 hmAb used for in vivo experiments was modified to increase half-life with M252Y/S254T/T256E (YTE) mutations.

For examples 11-16, African green monkey kidney epithelial cells (Vero E6, CRL-1586) were obtained from the American Type Culture Collection. A Vero E6 cell line expressing human ACE2 and TMPRSS2 (Vero AT) was obtained from BEI Resources (NR-54970). Cells were maintained in Dulbecco's modified Eagle medium (DMEM) supplemented with 5% (vol/vol) fetal bovine serum (FBS, VWR) and 1% penicillin-streptomycin-glutamine (PSG) solution (Corning). SARS-CoV-2 WA-1 (NR-52281), SARS-CoV-2 Beta (NR-54008), SARS-CoV-2 Gamma (NR54982), SARS-CoV-2 Delta (NR-55611), and SARS-CoV-2 Omicron (NR-56461); SARS-CoV, Urbani strain icSARS-CoV (NR-18925); and MERS-CoV, icMERS—CoV-RFP-ΔORF5 (NR48813) were obtained from BEI Resources. All natural isolate and recombinant SARS-CoV-2 viral stocks were completely sequenced.

For examples 11-16, ELISA plates (Nunc MaxiSorp; Thermo Fisher Scientific, Grand Island, NY) were coated with recombinant CoV proteins at 1 μg/ml. Recombinant proteins used include SARS-CoV-2 S2 (40590-V08B), SARS-CoV-2 S1 (40591-V08H3), SARS-CoV-2 S1+S2 (40589-V08B1), MERSCoV S2 (40070-V08), OC43 S2 (40607-V08B1), HKU1 S2 (40021-V08B) (Sino Biological, Wayne, PA), and SARS-CoV S (BEI Resources). Human plasma or purified hmAbs were diluted in PBS, and binding was detected with HRP-conjugated anti-human IgG (Jackson ImmunoResearch, West Grove, PA). In select ELISAs, 8M urea were added to the ELISA plate and the plates incubated for 15 min at room temperature prior to washing with PBS plus 0.05% Tween20 and detection with anti-IgG-HRP to evaluate avidity. Immunofluorescence assay was used to determine hmAb binding to SARS-CoV-2, SARS-CoV, or MERS-CoV infected cells. Briefly, confluent monolayers of Vero E6 cells were mock infected or infected with the indicated virus. At 24 hours post infection (hpi), cells were fixed with 4% paraformaldehyde (PFA) for 30 minutes and permeabilized with 0.5% Triton X-100-PBS for 15 min at room temperature, and blocked with 2.5% Bovine Serum Albumin at 37° C. for 1 h. Cells were then incubated for 1 h at 37° C. with 1 μg/ml of indicated hmAb. Then, cells were incubated with fluorescein isothiocyanate (FITC)-conjugated secondary anti-human Ab (Dako) for 1 h at 37° C. Images were captured using a fluorescence microscope and camera with a 10× objective.

For examples 11-16, experiments for BLI were performed on a Gator Prime instrument at 30° C. with shaking at 400-1000 rpm. All loading steps were 300s, followed by a 60s baseline in KB buffer (1X PBS, 0.002% Tween 20, and 0.02% BSA, pH 7.4), and then a 300s association phase and a 300s dissociation phase in K buffer. For the binding BLI experiments, mAbs were loaded at a concentration of 0.5 μg/mL in PBS onto Anti-Human IgG Fc capture (HFc) biosensors for a shift of 0.3 nm. After baseline, probes were dipped into five two-fold serial dilutions of Spike protein from SARS-CoV-2, SARS-CoV, or MERS (all from Acro Biosystems, Newark, DE) starting at 50 nM and a 0 nM for the association phase.

For examples 11-16, SPR experiments were performed on a Biacore T200 (Cytiva) at 25° C. using a running buffer consisting of 10 mM HEPES, 150 mM NaCl, 0.0075% P20. Competition SPR studies were performed by coupling S2-Frag4-murineFC protein to a CM-5 chip using a murine antibody capture kit (cytiva). Simultaneous injections (inject 1, inject 2) of hmAbs were performed at 50 nM each for 200 seconds at 20 μL/min. Binding responses were measured 60 seconds after inject 1 and inject 2. Raw hmAb sensorgram binding data (RU) collected during inject 1 were normalized to the amount of S2-Frag4-murineFC coupled (rubind/rucoupled) and defined as 100%. Raw RU hmAb binding after inject 2 was normalized as described above and defined as a percentage of hmAb binding recorded after inject 1. Kinetic binding analysis for 1249A8, CC40.8, S2P6, and CV3-25 were performed by capturing the hmAbs to the chip surface of CM-5 chips using a human antibody capture kit (cytiva). The binding kinetics for the interaction between hmAbs and SARS-CoV-2 Spike protein (R&D Systems, 10549-cv) was determined by injecting four concentrations of SARS-CoV-2 Spike (25 nM highest concentration) with a contact time of 240 seconds and a 300 second dissociation phase. The same parameters were used to characterize MERS-CoV S2 (Sino Biologicals, 40070-V08) binding to the hmAbs. All SPR experiments were double referenced (e.g., sensorgram data was subtracted from a control surface and from a buffer blank injection). The control surface for all experiments consisted of the capture antibody. Sensorgrams were globally fit to a 1:1 model, without a bulk index correction, using Biacore T-200 evaluation software version 1.0.

For examples 11-16, hmAbs were tested for neutralization of live SARS-CoV-2, SARS-CoV, and MERS-CoV. Vero E6 cells (96-well plate format, 4×104 cells/well, quadruplicate) were infected with 100-200 PFU/well of SARS-CoV-2. SARS-CoV-2 Omicron neutralization was performed in Vero AT using 600 PFU/well. After 1 h of viral adsorption, the infection media was changed with the 100 μl of post-infection media containing 1% Avicel and 2-fold dilutions, starting at 25 μg/ml of hmAb (or 1:100 dilution for human serum control). At 24 h p.i., infected cells were fixed with 10% neutral formalin for 24 h and were immune-stained using the anti-NP monoclonal antibody 1C7C7. Virus neutralization was evaluated using 3-4 replicates per mAb concentration and quantified using ELISPOT, and the percentage of infectivity calculated using sigmoidal dose response curves. The formula to calculate percent viral infection for each concentration is given as [(Average # of plaques from each treated wells-average # of plaques from “no virus” wells)/(average # of plaques from “virus only” wells-average # of plaques from “no virus” wells)]×100. A non-linear regression curve fit analysis over the dilution curve can be performed using GraphPad Prism to calculate NT50. Mock-infected cells and viruses in the absence of hmAb were used as internal controls. hmAbs were also tested using a SARS-CoV-2 Spike protein pseudotyped virus (PsV) expressing firefly luciferase. Virus neutralization was measured by the reduction of luciferase expression. VeroE6/TMPRSS2 cells were seeded at 2×10⁴ cells/well in opaque plates (Greiner 655083). The next day, PsV corresponding to 1-10×10⁶ luciferase units was mixed in Opti-MEM with dilutions of hmAbs and incubated at RT for 1 h. Media was removed from the cells and 100 l/well of the hmAb/PsV mix was added in triplicates. After 1 h incubation at 37° C. and 5% C02, another 100 μL of Opti-MEM was added, and cells were incubated for 24 more hours. After this time, luciferase activity was measured using Passive Lysis Buffer (Promega E1941) and Luciferase substrate (Promega E151A) following the manufacturer's instructions. Neutralization was calculated as the percent reduction of luciferase readings as compared to no-antibody-controls.

For examples 11-16, for measurement of the ADCP activity of the mAbs, SARS-CoV-2 Wuhan-Hu-1 Spike protein (NR-53524 BEI Resources) or MERS-CoV Spike protein (Sino Biological) was biotinylated with the Biotin-XX Microscale Protein Labeling Kit (Life Technologies, NY, USA). 0.25 μg of biotinylated Ag or ˜0.16 μg of BSA (used as a baseline control in an equivalent number of Ag molecules/bead) was incubated overnight at 4° C. with 1.8×10⁶ Yellow-Green neutravidin-fluorescent beads (Life Technologies) per reaction in a 25 μL of final volume. Antigen-coated beads were subsequently washed twice in PBS-BSA (0.1%) and transferred to a 5 mL Falcon round bottom tube (Thermo Fisher Scientific, NY, USA). mAbs, diluted at 5 μg/ml, were added to each tube in a 20 μL of reaction volume and incubated for a 2 h at 37° C. in order to allow Ag-Ab binding. Then 250,000 THP-1 cells (human monocytic cell line obtained from NIH AIDS Reagent Program) were added to the cells and incubated for 3 h at 37° C. At the end of incubation, 100 μL 4% paraformaldehyde was added to fix the samples. Cells were then assayed for fluorescent bead uptake by flow cytometry using a BD Biosciences Symphony. The phagocytic score of each sample was calculated by multiplying the percentage of bead positive cells (frequency) by the degree of phagocytosis measured as mean fluorescence intensity (MFI) and dividing by 10⁶. Values were normalized to background values (cells and beads without mAb) and an isotype control to ensure consistency in values obtained on different assays. Finally, the phagocytic score of the testing mAb was expressed as the fold increase over BSA-coated beads.

For examples 11-16, for experiments involving K18 hACE2 transgenic mice, five-week-old female K18 hACE2 transgenic mice were purchased from The Jackson Laboratory and maintained under specific pathogen-free conditions and ABSL3 containment. Mice were treated with a single dose of mAb delivered either i.p. or i.n. 1 day prior to viral challenge. For virus infection, mice were anesthetized following gaseous sedation in an isoflurane chamber and inoculated with viral dose of 105 PFU per mouse, intranasally. For ex vivo imaging of lungs, mice were humanely euthanized at 2 and 4 d p.i. to collect lungs. Fluorescent images of lungs were photographed using an IVIS (AMI HTX), and the brightfield images of lungs were taken using an iPhone 6s (Apple). Nasal turbinate and lungs from mock or infected animals were homogenized in 1 mL of PBS for 20 s at 7,000 rpm using a Precellys tissue homogenizer (Bertin Instruments). Tissue homogenates were centrifuged at 12,000×g (4° C.) for 5 min, and supernatants were collected and titrated by plaque assay and immunostaining as previously described. For the body weight and survival studies, five-week-old female K18 hACE2 transgenic mice were infected intranasally with 105 PFU per animal following gaseous sedation in an isoflurane chamber. After infection, mice were monitored daily for morbidity (body weight) and mortality (survival rate) for 11 d. Mice showing a loss of more than 25% of their initial body weight were defined as reaching the experimental end point and humanely euthanized. K18 hACE2 transgenic mice experiments were conducted once.

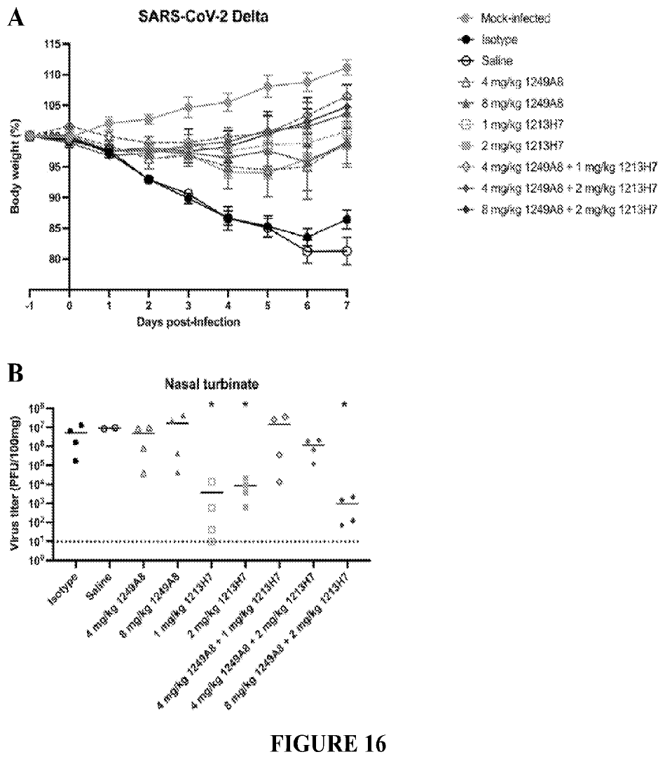

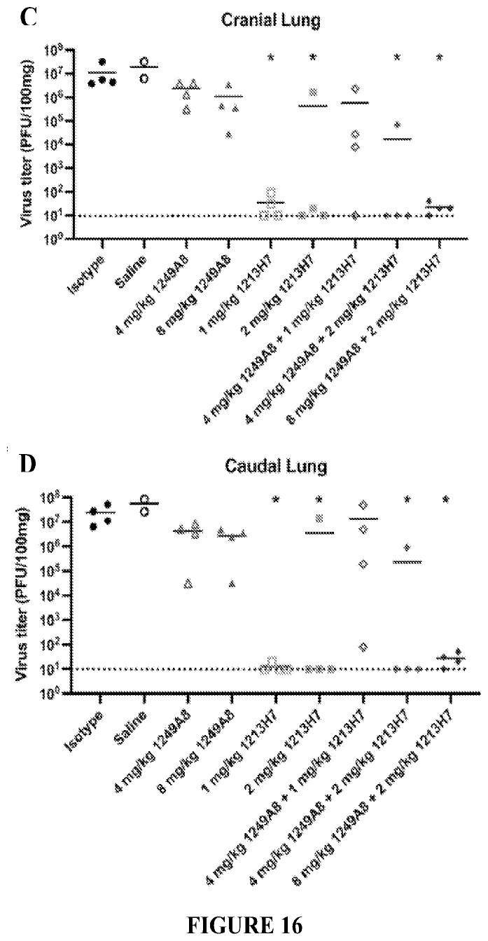

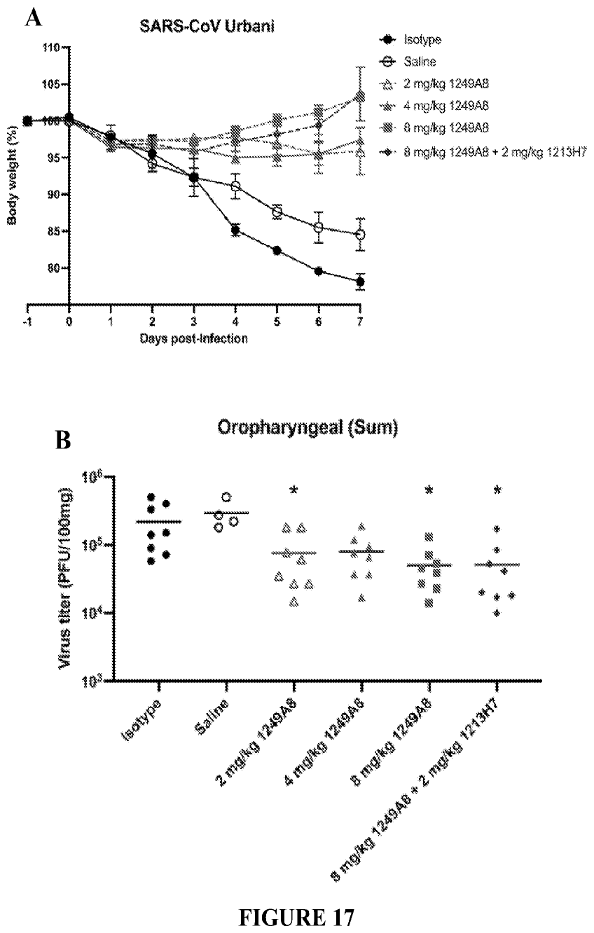

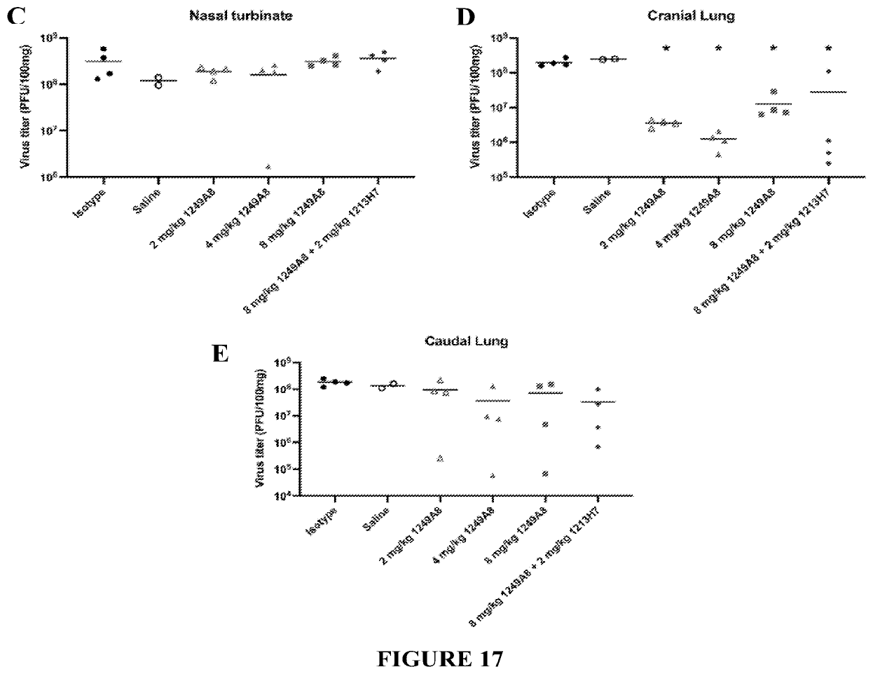

For examples 11-16, for experiments using Syrian hamsters, forty female Syrian hamsters (Envigo Corporation, Indianapolis, IN, USA) were housed in ventilated cages in separate rooms under ABSL3 containment. They were acclimated for 7-14 days after arrival and inoculated with virus at 6 weeks of age. Animals were challenged with virus under ketamine-xylazine anesthesia by intranasal instillation of 100 ul of virus diluted in PBS to achieve a dose of 104 cell culture infectious dose 50% (CCID50) ((rSARS-CoV-2 Delta B.1.617.2 (hCoV-19/USA/CA-VRLC086/2021) BEI Resources (Manassas, VA, USA) or plaque-forming units (SARS-CoV, Urbani Strain; BEI Resources); the inocula were back-titrated after completion of the challenge to confirm dose delivered. Antibody therapies were administered as summarized in FIGS. 16 and 17 at 12 hours following virus inoculation, again under ketamine-xylazine anesthesia and by intranasal instillation of 100 ul. Oropharyngeal swabs were collected daily from all hamsters on days 1, 2 and 3 postvirus inoculation. Swabs were broken off into 1 ml of BA1 medium (Tris-buffered minimal essential medium containing 1% BSA) supplemented with 5% fetal bovine serum (BA1-FBS) and stored at −80° C. until assay. Half of the hamsters inoculated with virus were euthanized on day 3 and half on day 7 post-challenge. For the animals euthanized on day 3, samples of nasal turbinates and cranial and caudal right lung were homogenized in BA1-FBS using a mixer mill and stainless-steel balls to obtain ˜10% tissue homogenates. Infectious virus in tissue homogenates and oropharyngeal swabs was titrated by double-overlay plaque assay. Briefly, 10-fold serial dilutions of samples were prepared in BA1 medium with antibiotics, inoculated onto confluent monolayers of Vero cells in 6-well plates, incubated with rocking for 45 minutes, and then overlaid with 0.5% agarose in phenol-red free MEM supplemented with antibiotics. Plates were incubated for one (SARS CoV) or two (SARS-CoV-2) days and second overlay containing neutral red dye was added. Plaques were counted 1 day after the second overlay. The limit of detection for this assay was 10 PFU/swab and 100 PFU/gram of tissue. For each animal, the viral titer from their D1, D2, and D3 oropharyngeal swabs was combined to provide a summed value. Golden Syrian hamster experiments were conducted once.

### Example 12: S2 hmAbs have In Vitro SARS-CoV-2 Neutralizing and Antibody-Dependent Phagocytosis Activity

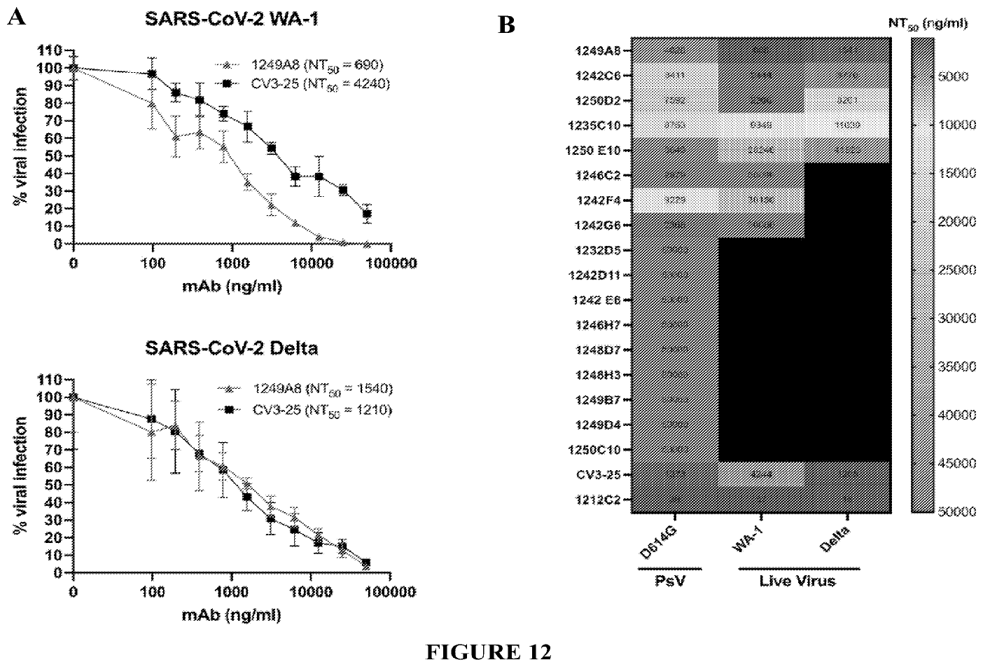

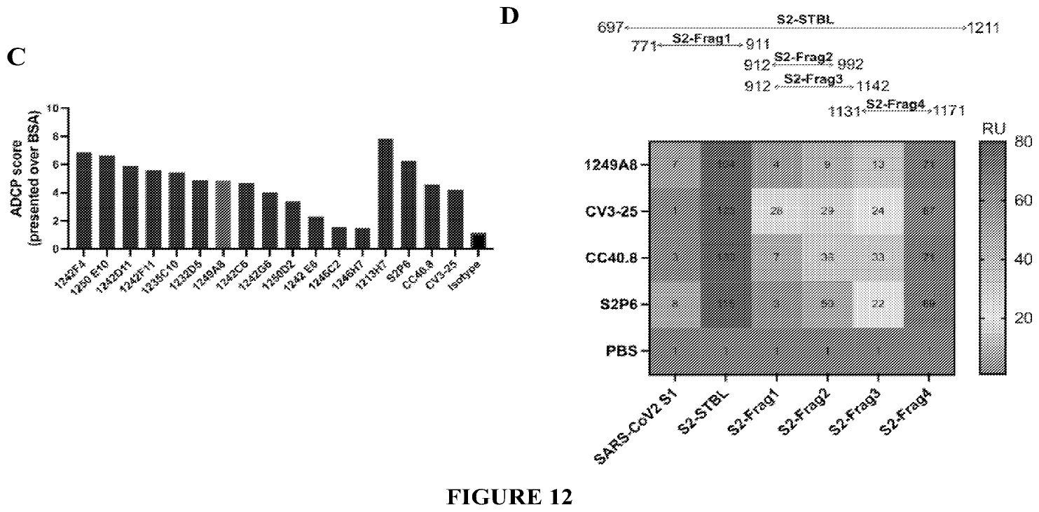

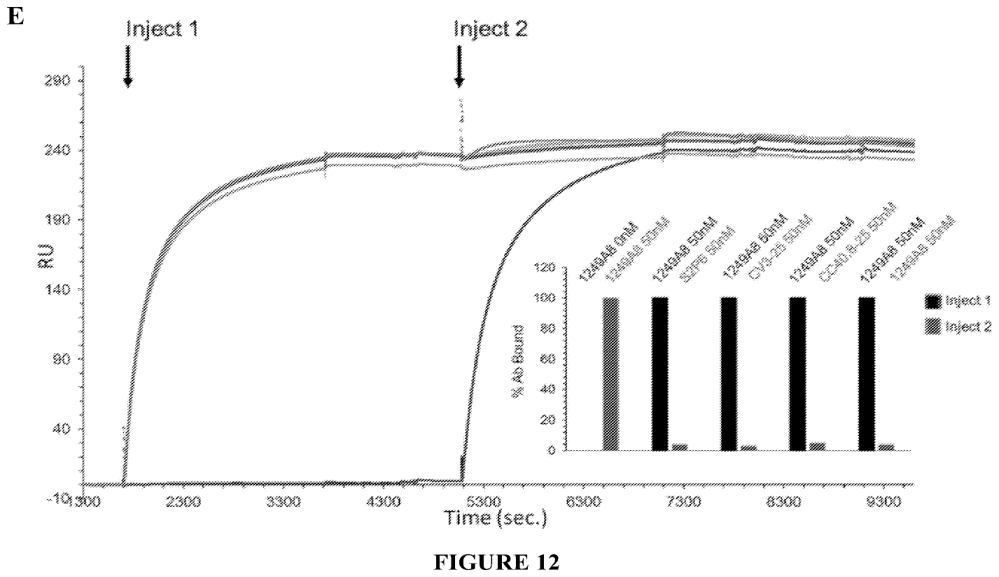

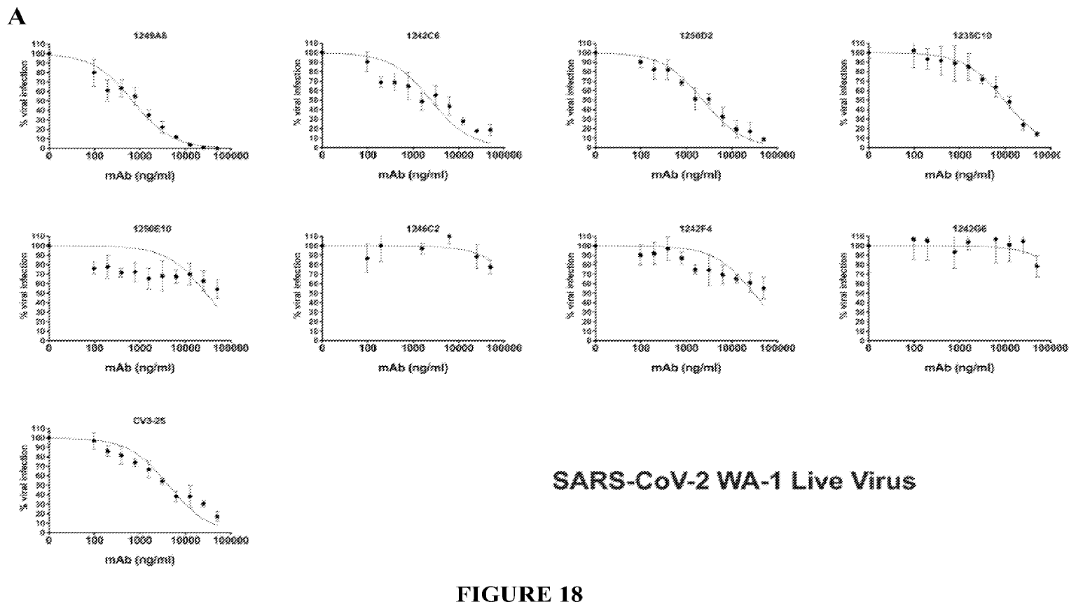

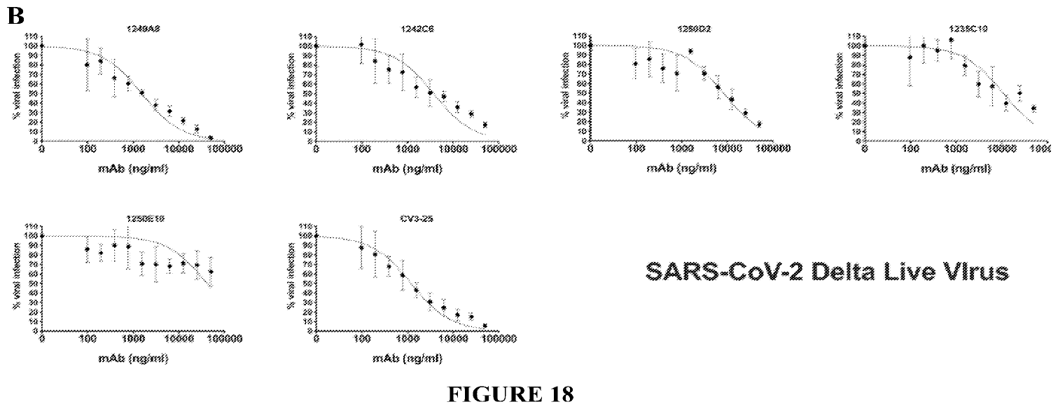

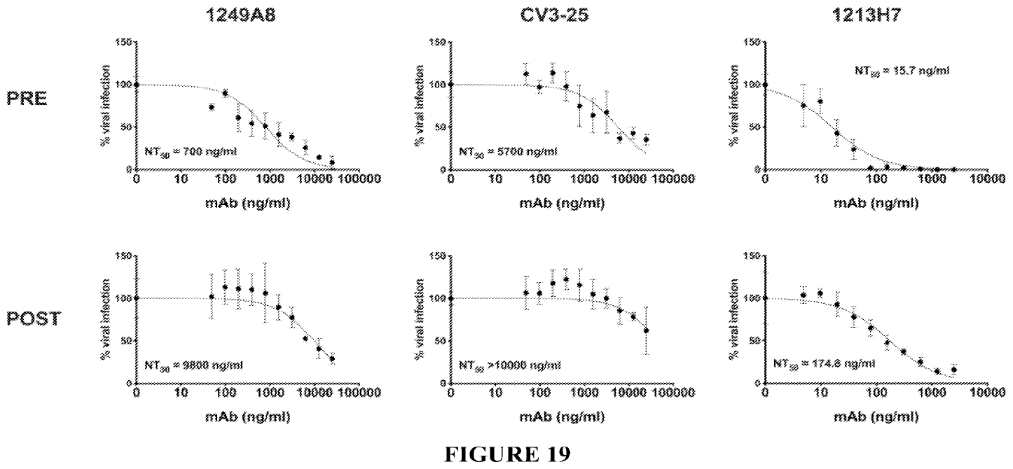

The functional activity of S2 hmAbs against SARS-CoV-2 was tested and previously reported S2 hmAbs were included as controls. The hmAbs that showed the greatest binding to at least one S2 protein by ELISA were tested by live virus and pseudovirus-based neutralization assays (FIGS. 12A and 12B and 18 and 19). Several hmAbs did not show neutralization capacity, even at the highest concentration (50 μg/ml). Eight hmAbs demonstrated neutralization of SARSCoV-2 D614G pseudovirus (PsV) and were tested further, of which four hmAbs (1249A8, 1242C6, 1250D2, and 1235C10) effectively neutralized both PsV and live virus including Delta VoC. 1249A8 emerged as having the broadest and most potent neutralizing activity, with comparable NT₅₀ (neutralization titer at 50% inhibition) to CV3-25, a previously described S2 neutralizing hmAb.

The Fc effector function of the S2 hmAbs was assessed by antibody dependent cellular phagocytosis (ADCP) of SARS-CoV-2 Wuhan-Hu-1 Spike coated beads (FIG. 12C). 1242F4 and 1250E10 had the highest ADCP activity, similar to the previously described S2-specific hmAb S2P6. Both 1246C2 and 1246H7 had activity that was only slightly higher than the isotype control indicating very little Fc effector function, and consistent with their limited binding and neutralizing activity. 1249A8 had 4.2 fold greater ADCP activity than the isotype control which was similar to the S2 hmAbs CC40.8 and CV3-25. The SARS-CoV-2 RBD specific mAb 1213H7, had the greatest (˜8 times greater than isotype) ADCP activity. Together these results suggest S2 hmAbs have the potential to eliminate SARS-CoV-2 through both neutralization and Fc-dependent effector functions. Based on the potent neutralizing activity of 1249A8, Applicants sought to determine the location of its binding to S2. Four S2 protein fragments (S2 Frag1-Frag4) that cover different regions of the S2 amino acid sequence were produced to approximate the region containing the epitope of 1249A8 (FIG. 12D). S2 fragment binding assays localized the 1249A8 binding epitope to S2 residues 1131-1171 (S2-Frag4), which contains the conserved stem helix region (residues 1148-1158) of S2, previously reported to be recognized by mAbs CV3-25, CC40.8, and S2P6. Competition surface plasmon resonance (SPR) analysis revealed that 1249A8 prevents the binding of CV3-25, CC40.8, and SP26 to S-Frag4 (FIG. 12E) indicating they recognize an overlapping epitope.

### Example 13: Molecular Characteristics of S2 hmAbs

The most potent neutralizing hmAb, 1249A8 was isolated from an IgG1 expressing B cell and exhibited substantial somatic hypermutation including 16.73 amino acid mutation from germline in the heavy chain variable region, and 13.5% amino acid mutation from germline in the light chain variable region (Table 7). The 1249A8 hmAb is a member of the same clonal lineage that includes 1242C6, 1250D2, 1242F4, 1249D4, and 1249B7. This shared lineage utilizes V_{H}1-46 heavy chain gene and Vκ3-20 light chain gene, with all members isolated from IgG1 expressing B cells and demonstrating substantial somatic hypermutation in the heavy (15.6-18.8% AA) and light (10.4-13.5% AA) from germline.

TABLE 7

| | Native | | | | Mutation | | | Mutation |
|---|---|---|---|---|---|---|---|---|
| hmAb | isotype | VH | DH | HI | (% NT/% AA) | VL | JL | (% NT/% AA) |
| 1249A8 | IgG1 | VH1-46 | DH1-26 | JH4 | 9.7/16.7 | VK3-20 | JK2 | 7.6/13.5 |
| 1242C6 | IgG1 | VH1-46 | DH1-26 | JH4 | 9.7/16.7 | VK3-20 | JK2 | 6.2/12.5 |
| 1250D2 | IgG1 | VH1-46 | DH1-26 | JH4 | 9.0/16.7 | VK3-20 | JK2 | 5.5/10.4 |
| 1242F4 | IgG1 | VH1-46 | DH3-16 | JH4 | 12.4/18.8 | VK3-20 | JK2 | 6.2/10.4 |
| 1249D4 | IgG1 | VH1-46 | DH1-26 | JH4 | 7.6/15.6 | VK3-20 | JK2 | 6.9/12.5 |
| 1249B7 | IgG1 | VH1-46 | DH1-26 | JH4 | 9.0/17.7 | VIC3-20 r | JK2 | 6.6/13.5 |
| 1250E10 | IgG1 | VH1-46 | DH1-26 | JH5 | 13.4/20.8 | VK3-20 | JK2 | 9.7/16.7 |
| 1242D11 | IgAl | VH3-7 | DH3-22 | JH2 | 2.0/4.1 | VK3-20 | JK2 | 2.1/4.2 |
| 1242E6 | IgG1 | VH3-7 | DH3-9 | JH5 | 1.0/1.0 | VL2-14 | JL1 | 4.0/6.1 |
| 1246C2 | IgG3 | VH3-21 | DH1-26 | JH2 | 7.4/14.3 | VK3-11 | JK4 | 6.3/9.5 |
| 1232D5 | IgG1 | VH3-30 | DH6-19 | JH4 | 2.7/5.2 | VL1-44 | JL3 | 2.4/4.1 |
| 1242G6 | IgG1 | VH3-30 | DH3-10 | JH4 | 10.1/19.4 | VK1-9 | JK5 | 5.3/10.5 |
| 1235C10 | IgG1 | VH3-49 | DH3-10 | JH4 | 2.0/3.0 | VK4-1 | JK1 | 2.0/5.0 |
| 1246H7 | IgM | VH4-39 | DH3-16 | JH4 | 4.1/6.1 | VL2-11 | JL3 | 3.4/4.1 |

### Example 14: 1249A8 hmAb Protects from SARS-CoV2 Infection

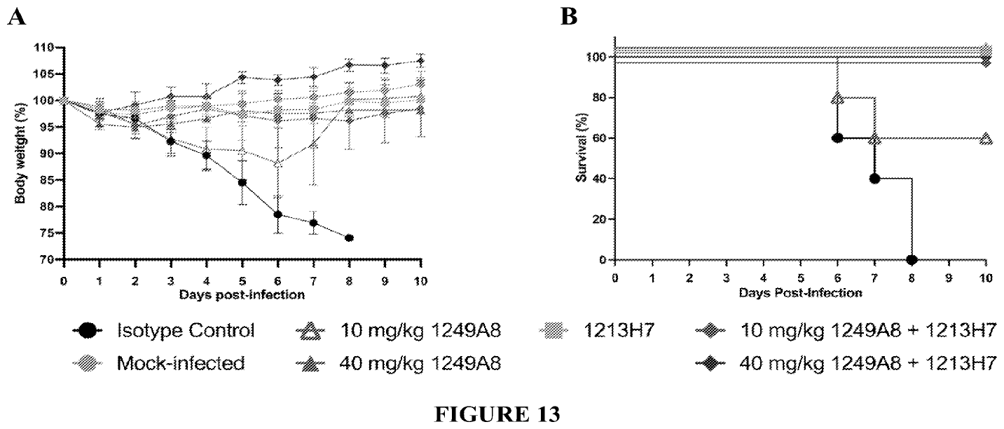

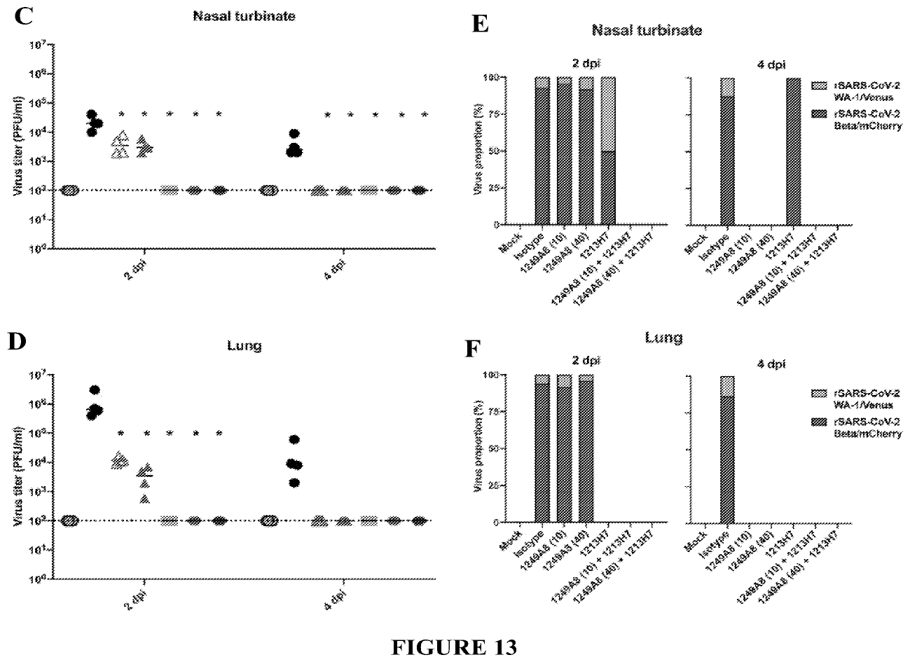

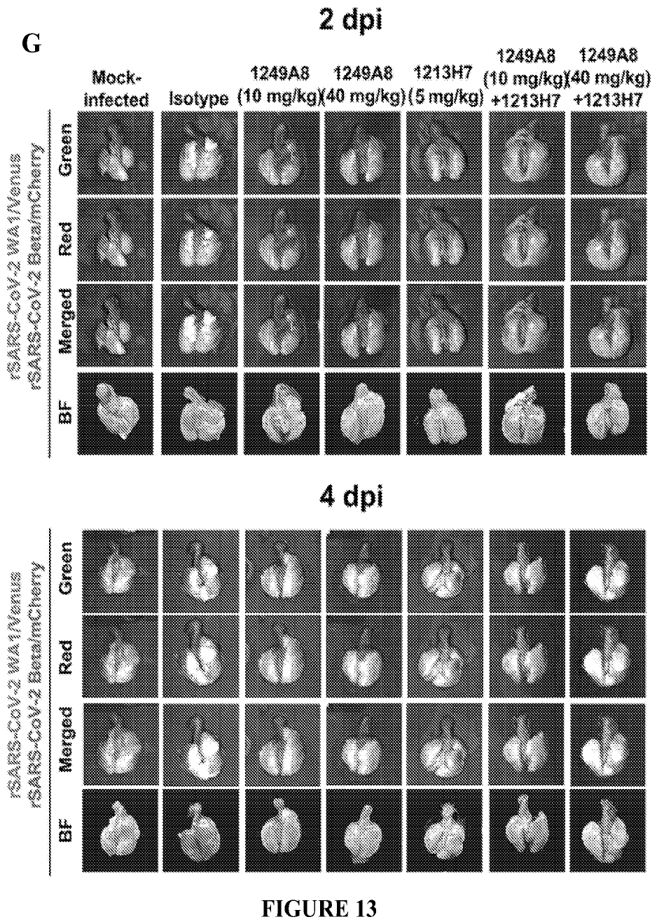

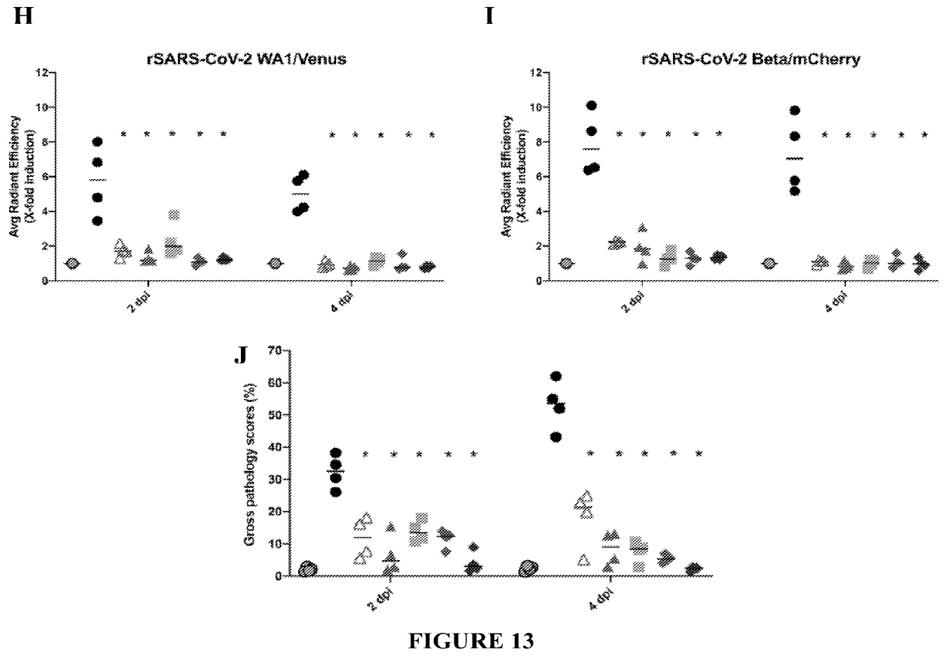

The K18 human ACE2 transgenic mouse model was utilized to determine the prophylactic activity of 1249A8 hmAb. Mice were treated with a single dose of 1249A8 intraperitoneally (IP), and 12 hours later challenged with both rSARS-CoV-2 WA-1/Venus and rSARS-CoV-2 Beta/mCherry reporter viruses. Infecting animals with both viruses enables efficient assessment of invivobreadth of the mAb activity. 12498 was administered at 10 and 40 mg/kg, doses chosen based on relatively higher NT50 of S2 mAbs compared to well described RBD-specific mAbs. Mice were also treated alone or in combination with a modest dose of 12131H7 (5 mg/kg), a broad and potent SARS-CoV-2 RBD specific hmAb. All mice treated with the isotype control hmAb had declining body weight following infection that required euthanasia before D9 (FIGS. 13A and 13B). Mice treated with 10 mg/kg 1249A8 showed a milder weight loss with 60% of the mice surviving. Mice treated with 40 mg/kg of 1249A8 prior to infection, as well as those treated with 12131H7 or the combination of both did not have weight loss and all survived. Both 10 mg/kg and 40 mg/kg 1249A8 significantly (p<0.05) reduced nasal virus at day (D) 2, with 1249A8 and 12131H7 combination treated mice not having detectable virus, with all 1249A8 treated mice having viral titer below the limit of detection at D4 (FIG. 13C), with viral burden dominated by rSARS-CoV-2 Beta/mCherry (FIGS. 13E and 13F). Lungs of mice that were treated with the isotype control mAb showed intense fluorescent radiance for both rSARS-CoV-2 WA-1/Venus and rSARS-CoV-2 Beta/mCherry in left and right hemispheres by D2 following infection and markedly increased at D4, and minimally visually evident in the 1249A8, 1213H7, and combination treated mice (FIG. 13G). Lung viral titer was reduced in mice treated with either 1249A8 doses by ˜2 log at D2, and to below detection limit at D4 compared to isotype control hmAb treated mice (FIG. 13D). The reduction in viral burden by 1249A8 was for both rSARS-CoV2 WA-1/Venus and rSARS-CoV2 Beta/mCherry (FIGS. 13G-13I) and was consistent with significant (p<0.05) reduction in lung pathology (FIG. 13J). Overall, the RBD specific hmAb 12137H7 was very potent at reducing viral burden alone, resulting in difficulty resolving superiority for the combination. These results do indicate that 1249A8 alone and in combination with the RBD mAb 1213H7 can broadly limit SARS-CoV-2 upper and lower respiratory viral burden and lung pathology, including the original lineage A and the lineage B Beta VoC.

### Example 14: S2 hmAbs have Broad β-Coronavirus In Vitro Activity

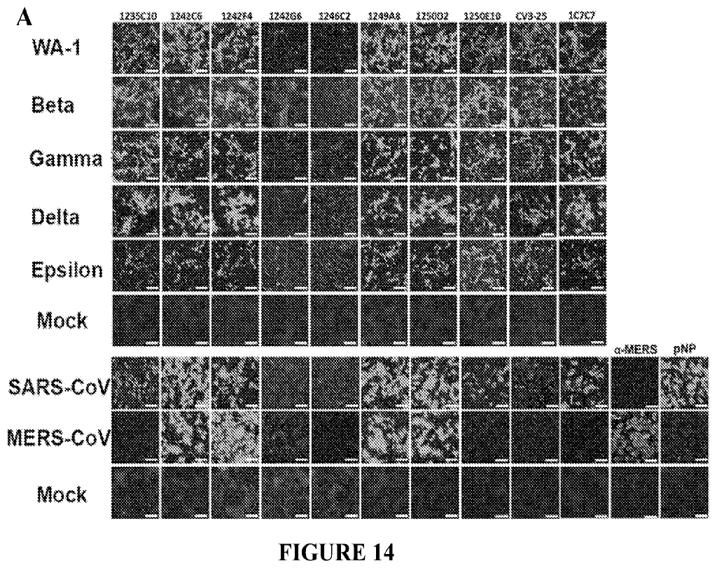

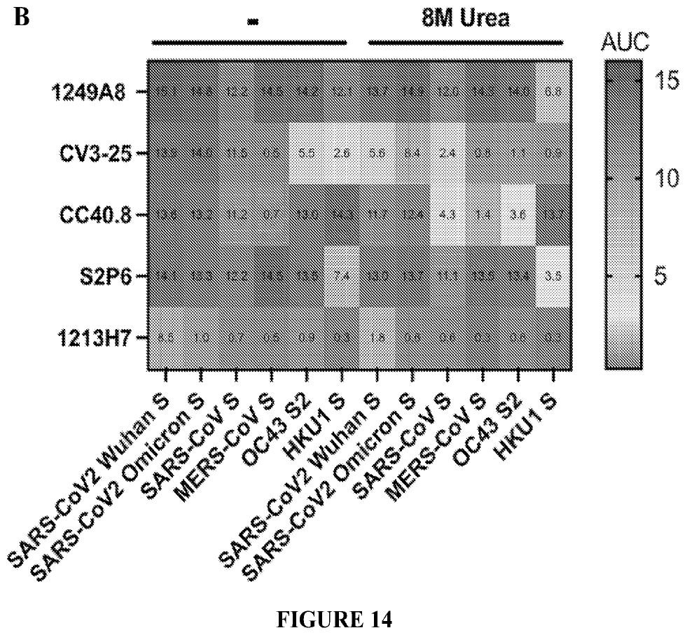

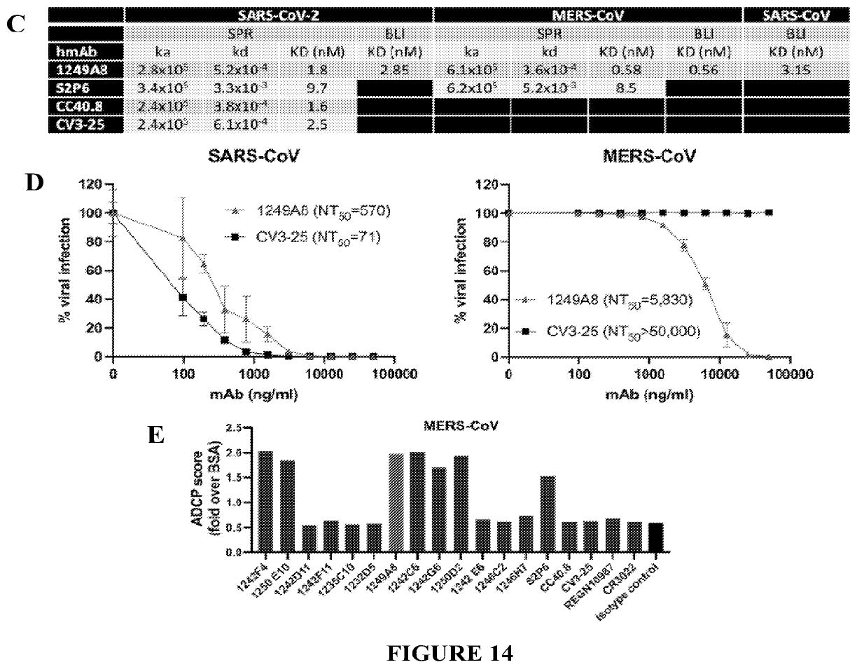

Given the high conservation of S2 across CoV, the S2 hmAbs were evaluated for their binding and neutralization breadth against diverse SARS-CoV-2 variants and CoV. Vero E6 cells were infected with SARS-CoV-2, SARS-CoV-2 variants, SARS-CoV, and MERS-CoV and binding assessed by immunofluorescence assay (IFA). Six of the hmAbs showed binding to all SARSCoV-2 isolates, however the hmAb 1242G6 and 1246C2 bound poorly to SARS-CoV-2 infected cells (FIG. 14A), consistent with their weak neutralizing activity. 1242C6, 1242F4, 1249A8 and 1250D2 all bound to SARS-CoV and MERS-CoV infected cells. CV3-25 had limited binding to SARS-CoV infected cells and no binding to MERS-CoV infected cells.

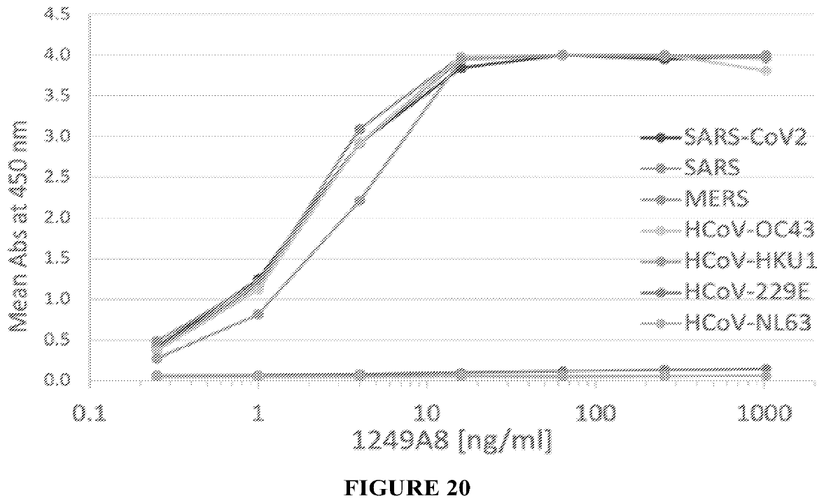

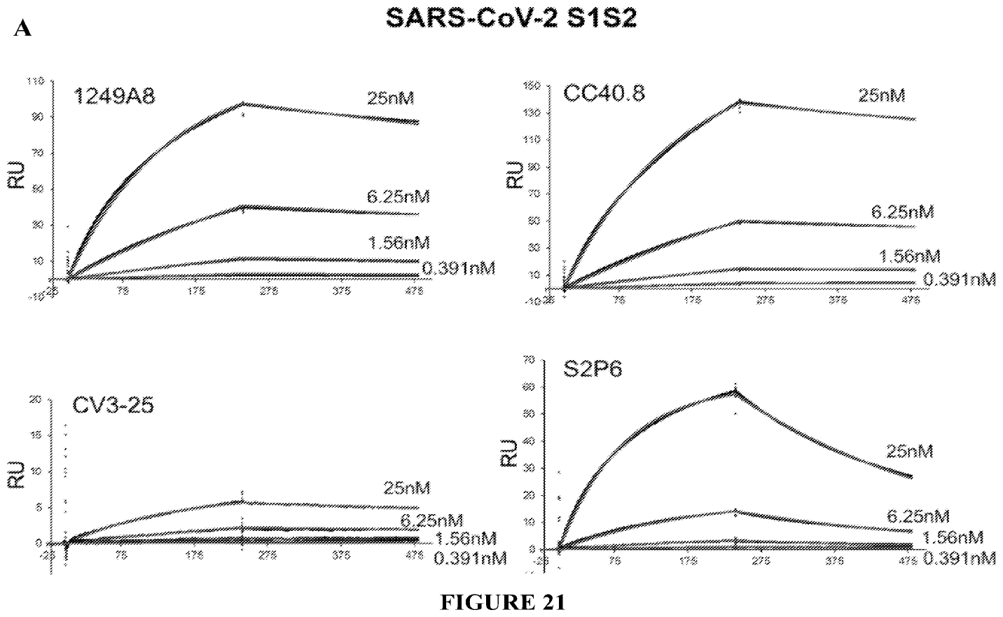

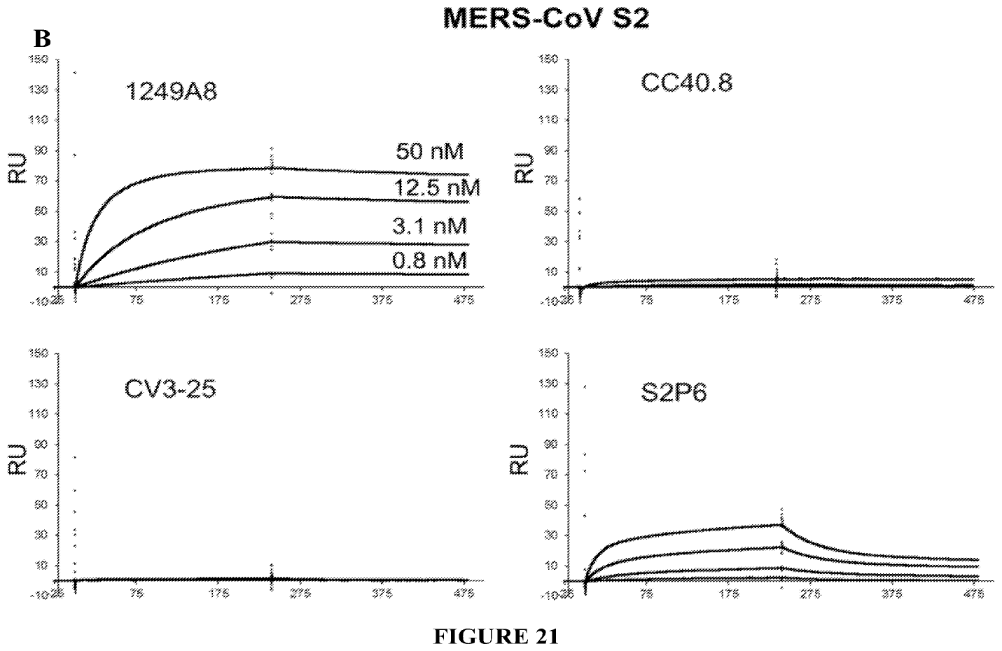

The breadth of the S2 binding activity of 1249A8 was further evaluated by avidity ELISA in the presence of urea, confirming its binding to SARS-CoV and MERS-CoV Spike, and also demonstrating its binding to OC43 and HKU-1 seasonal β-CoV (FIG. 14B). No binding to the seasonal α-CoV 229E or NL63 Spike proteins was detected (FIG. 20). 1249A8 uniquely had substantial broad binding to recombinant S proteins from MERS-CoV, OC43, and HKU1 compared to CV3-25 and CC40.8 which did not recognize MERS-CoV S, and S2P6 which had lower reactivity to HKU-1 S compared to 1249A8 (FIG. 14B). Consistent with the ELISA data, SPR/BLI studies show 1249A8 exhibits high affinity for SARS-CoV-2 (KD=1.8 nM), SARSCoV (KD=3.2 nM) and MERS-CoV (KD=0.58 nM) Spikes. 1249A8 had higher affinity to SARS-CoV-2 and MERS-CoV Spikes as compared to S2P6, as a result of a S2P6 having a faster off-rate (kd) (FIGS. 14C and 21). 1249A8 effectively neutralizes both live SARS-CoV (NT₅₀=570 ng/ml) and MERS-CoV (NT₅₀=5,830 ng/ml) (FIG. 14D). CV3-25 did not neutralize MERS-CoV. Additionally, 1249A8 has ADCP activity against MERS-CoV Spike coated beads (FIG. 14E). These results indicate that several SARS-CoV-2 S2 specific hmAbs have broad beta-CoV reactivity, with 1249A8 demonstrating universal β-CoV functional activity.

### Example 15: In Vitro and In Vivo Activity of Combined S1 and S2 Neutralizing mAbs Against SARS-CoV-2 Omicron

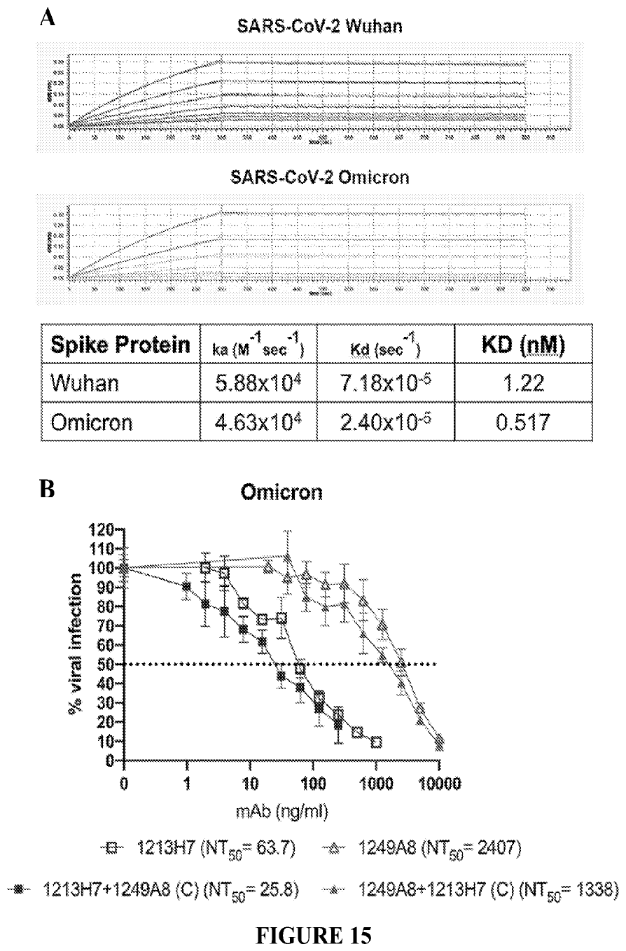

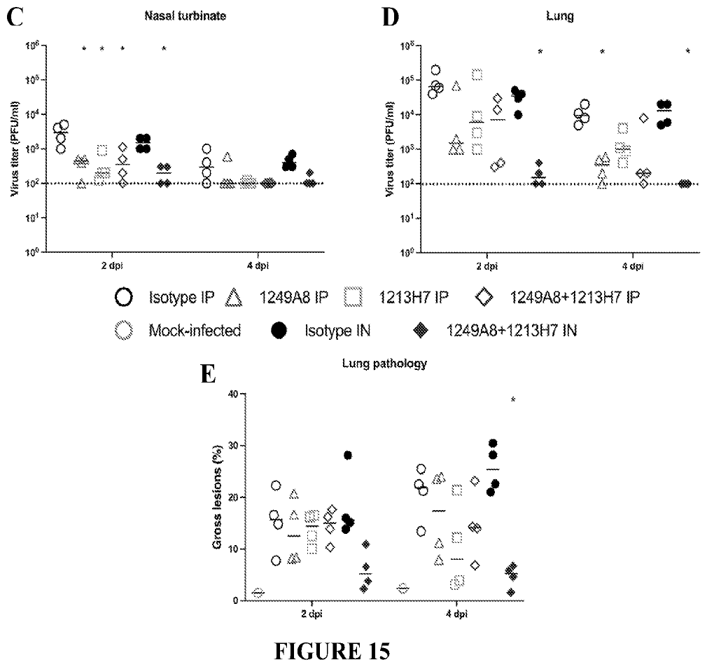

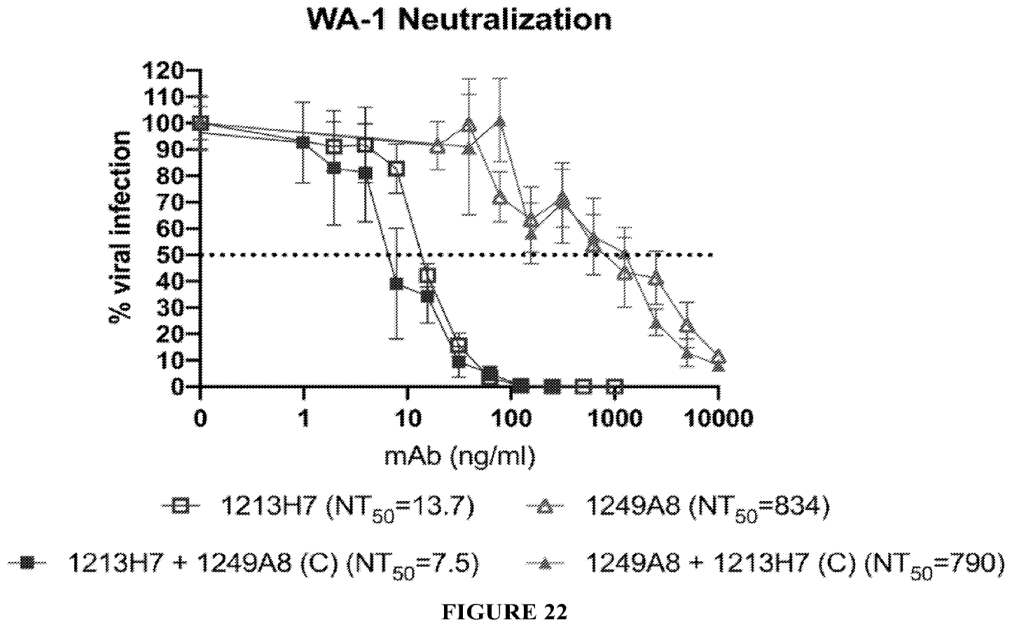

The emergence of the SARS-CoV-2 Omicron VoC and its substantial evasion of neutralizing antibodies necessitated testing of 1249A8. 1249A8 retains high affinity (KD=0.52 nM) for the SARS-CoV-2 Omicron Spike (FIG. 15A) and neutralizing activity (NT₅₀=2407 ng/ml) against live SARS-CoV-2 Omicron virus (FIG. 15B). Applicants also observed potent SARS-CoV-2 Omicron neutralization of 1213H7 (NT₅₀=64 ng/ml). As clinical development of an S2 mAb would likely include a RBD specific mAb, and as these mAbs target distinct Spike domains (S1 and S2) and steps in the infection process (attachment and fusion) Applicants sought to determine their combinatorial activity. In the presence of 50 ng/ml 1213H7, the NT₅₀ of 1249A8 against SARS-CoV-2 Omicron was reduced to 1338 ng/ml, and complementarily, in the presence of 2000 ng/ml of 1249A8 the NT₅₀ of 1213H7 was reduced to 26 ng/ml, with similar effect observed for SARS-CoV-2 WA-1 (FIG. 22), suggesting co-operative activity of the S2 and RBD mAbs in neutralizing SARS-CoV-2. Treatment of K18 hACE2 mice with 1249A8 alone i.p. (40 mg/kg) prior to challenge with 10⁵ plaque forming units (PFU) of SARS-CoV-2 Omicron significantly reduced upper and lower respiratory viral burden compared to isotype control treated mice (FIGS. 15C and 15D). Treatment with 1213H7 alone i.p. (10 mg/kg) significantly reduced upper respiratory viral burden. The combination of 1249A8 and 1213H7 significantly reduced viral burden, being more pronounced when the hmAbs were administered directly to the respiratory tract through intranasal (i.n.) delivery, with 50% of the mice not having detectable virus in the nasal turbinate at D2 and D4, and lungs at D2. None of the mice treated i.n. with the 1249A8 and 1213H7 combination had detectable virus in the lungs at D4. This was consistent with the significant reduction in lung pathology in these mice (FIG. 15E). These results indicate that direct respiratory administration of the RBD and S2 mAb cocktail of 1213H7 and 1249A8, respectively significantly reduce SARS-CoV-2 Omicron viral burden.

### Example 16: Direct Respiratory Administration of 1249A8 has Broad β-Coronavirus Therapeutic Activity

Given the broad β-CoV activity of 1249A8 in vitro and its demonstrated prophylactic activity against SARS-CoV-2 WA-1, Beta, and Omicron in K18 hACE2 mice, Applicants evaluated its therapeutic potential in hamsters when delivered directly to the respiratory tract. Hamsters were infected with SARS-CoV-2 Delta and 12 h p.i. were treated with a single dose of hmAb delivered intranasally. Isotype control hmAb treated and untreated hamsters exhibited ˜15% body weight loss within 6 days post infection (d p.i.), with minimal weight loss in hamsters treated with the 1249A8 or 1213H7 alone, or in combination (FIG. 16A). Treatment with 1249A8 alone had minimal impact on nasal virus titer at D3, although 1213H7 alone, and combined treatment with 8 mg/kg of 1249A8 and 2 mg/kg 1213H7, significantly reduced upper respiratory viral burden by ˜3 logs (FIG. 16B) and lower respiratory viral burden by ˜6 logs (FIGS. 16C and 16D) compared to control groups.

To assess pan β-CoV therapeutic activity, hamsters were infected with SARS-CoV, Urbani strain, and then 12 h p.i. treated similarly with a single dose of hmAb delivered intranasally. 1213H7 has minimal binding to SARS-CoV Spike, but the combination therapy group was included as it may represent a future clinical formulation. Untreated hamsters and those treated with isotype control hmAb lost 15 to 20% of body weight by 7 d p.i. Hamsters treated with 2, 4 or 8 mg/kg of 1249A8 had <5% weight loss, and those treated with 8 mg/kg 1249A8 alone or in combination with 2 mg/kg of 1213H7 actually gained weight by 7 d p.i. (FIG. 17A). A significant reduction in upper respiratory viral burden between D1-D3 as determined by oropharyngeal swabbing daily was evident in animals treated with 2 or 8 mg/kg of 1249A8 alone and in combination with 2 mg/kg of 1213H7 (FIG. 17B). At D3 minimal reduction in nasal turbinate virus (FIG. 17C) was evident, although a reduction in lung viral titer was evident in hamsters treated with 1249A8 alone and in combination with 1213H7 that was significant in the cranial lung (FIG. 17D), but not as apparent in the caudal lung (FIG. 17E). These results indicate the S2 hmAb 1249A8 has broad β-CoV in vivo therapeutic activity.

1. An isolated antibody, comprising isolated antibody 1249A8, or an antigen-binding fragment thereof, that specifically binds to a Coronavirus spike protein.

2. An isolated antibody, or an antigen-binding fragment thereof, that specifically binds to a spike protein of SARS-CoV-2 selected from the group consisting of: 1212C2, 1213H7, 1232D5, 1235C10, 1242C6, 1242D11, 1242E6, 1242F4, 1242F11, 1242G6, 1246C2, 1246H7, 1249A8, 1250D2, and 1250E10 variant.

3. The at least one isolated antibody, or the antigen-binding fragment thereof, of claim 2, wherein the at least one isolated antibody, or the antigen-binding fragment thereof, comprises 1213H7 and 1249A8.

4. (canceled)

5. An isolated antibody, or an antigen-binding fragment thereof, that specifically binds to a spike protein of SARS-CoV-2 comprising: (i) a heavy chain variable region comprising the amino acid sequence selected from the group consisting of SEQ ID NOS: 01, 03, 05, 07, 09, 11, 13, 15, 17, 19, 21, 23, 25, 27 and 29, or an amino acid sequence at least 80% identical thereto and (ii) a light chain variable region comprising the amino acid sequence selected from the group consisting of SEQ ID NOS: 02, 04, 06, 08, 10, 12, 14, 16, 18, 20, 22, 24, 26, 28 and 30, or an amino acid sequence at least 80% identical thereto.

6. The isolated antibody, or the antigen-binding fragment thereof, of to 5, wherein the antibody, or antigen binding fragment thereof, is an antibody variant.

7-10. (canceled)

11. The isolated antibody, or the antigen-binding fragment thereof, of claim 5, wherein the antibody, or antigen binding fragment thereof, is a chimeric antibody, a class-switched antibody, or a monoclonal antibody.

12. (canceled)

13. The isolated antibody, or the antigen-binding fragment thereof, of claim 5, wherein the antigen binding fragment is selected from the group consisting of: a Fab fragment, a F(ab)2 fragment, a Fab′ fragment, a Fd fragment, a Fv fragment, a disulfide-linked Fv, a dAb fragment, an isolated CDR, a nanobody, a single domain antibody, a portion of the V_{H} region containing a single variable domain and two constant domains, a diabody, a triabody, a tetrabody, a scFv, a scFv-FC, a scFv-CH, a scFab, and a scFv-zipper.

14. The isolated antibody, or the antigen-binding fragment thereof, of claim 5, wherein the antibody, or antigen binding fragment thereof, binds an epitope within the receptor binding domain of the SARS-Co V-2 spike protein, binds an epitope within the receptor binding motif of the SARS-Co V-2 spike protein, or binds an epitope within the S2 region of the SARS-CoV-2 spike protein.

15. The isolated antibody, or the antigen-binding fragment thereof, of claim 5, wherein the antibody, or antigen binding fragment thereof, reduces binding of SARS-CoV-2 to a target cell, reduces cellular fusion between SARS-CoV-2 and the target cell, reduces release of infective SARS-CoV-2 from an infected cell, reduces infection of the target cell by SARS-CoV-2, or a combination of the foregoing.

16. An isolated nucleic acid encoding the heavy chain variable region, the light chain variable region, or both the heavy chain variable region and the light chain variable region of the antibody, or antigen binding fragment thereof, of claim 5.

17. An expression vector comprising the nucleic acid of claim 16.

18. A cultured host cell comprising the expression vector of claim 17.

19. A pharmaceutical composition comprising the antibody, or the antigen-binding fragment thereof, of claim 5 and at least one of a pharmaceutically acceptable carrier and a pharmaceutically acceptable excipient.

20. (canceled)

21. A method for treating a SARS-CoV-2 infection in a subject, the method comprising administering to said subject an effective amount of at the antibody, or antigen-binding fragment thereof, of claim 5.

22-27. (canceled)

28. A method of reducing cellular entry of SARS-CoV-2 in a subject, the method comprising administering to said subject an effective amount of at the isolated antibody, or an antigen-binding fragment thereof, of claim 5.

29-34. (canceled)

35. A method of reducing binding of SARS-Co V-2 to a cellular ACE2 in a subject, the method comprising administering to said subject an effective amount of the isolated antibody, or an antigen-binding fragment thereof, of claim 5.

36-41. (canceled)

42. A method for reducing viral titer of a SARS-CoV-2 in a bodily fluid, tissue or cell of a subject, the method comprising, administering to said subject an effective amount of the isolated antibody, or an antigen-binding fragment thereof, of claim 5.

43-48. (canceled)

49. A method for reducing the transmission of a SARS-CoV-2 infection from a first subject to a second subject, the method comprising administering to said first subject an effective amount of the isolated antibody, or an antigen-binding fragment thereof, of claim 5.

50-55. (canceled)

56. A method for reducing the transmission of a SARS-CoV-2 infection from a first subject to a second subject, the method comprising administering to the second subject an effective amount of the isolated antibody, or an antigen-binding fragment thereof, of claim 5.

57-62. (canceled)

63. A method of neutralizing a SARS-CoV-2 in a subject, the method comprising: administering to said subject an effective amount of the isolated antibody, or an antigen-binding fragment thereof, of claim 5.

64-74. (canceled)
